(19) United States
(12) Patent Application Publication
(10) Pub. No.: US 2002/0151712 A1
(43) Pub. Date: Oct. 17, 2002

(54) 3-pyrrolidinyloxy-3'-pyridyl ether compounds useful for controlling chemical synaptic transmission

(76) Inventors: Nan-Horng Lin, Vernon Hills, IL (US); Yihong Li, Carmel, IN (US); Irene Drizin, Wadsworth, IL (US); John F. Kincaid, Newbury Park, CA (US); Anwer Basha, Lake Forest, IL (US); Liming Dong, San Diego, CA (US); Ahmed A. Hakeem, Niles, IL (US)

Correspondence Address: ABBOTT LABORATORIES, DEPT. 377 - AP6D-2, 100 ABBOTT PARK ROAD, ABBOTT PARK, IL, 60064-6050, US

(21) Appl. No.: 10/100,764

(22) Filed: Mar. 19, 2002

Related U.S. Application Data
(63) Continuation of application No. 09/661,710, filed on Sep. 14, 2000, now abandoned.
(60) Provisional application No. 60/153,894, filed on Sep. 14, 1999.

(51) Int. Cl. C 07D 4 1/02; C07D 207/04; C07D 205/00; C07D 223/12; C07D 223/04

(52) U.S. Cl. 540/450; 540/605; 540/610; 546/207; 546/229; 546/238; 548/950; 548/566; 548/572

(57) ABSTRACT

A series of 3-pyrrolidinyloxy-3′-pyridyl ether compounds, a method for selectively controlling neurotransmitter release in mammals using these compounds, and pharmaceutical compositions including these compounds. Preferred compounds are 3-pyrrolidinylmethoxy-3′-(5′-and/or 6′-substituted) pyridyl ethers.

This application is a continuation application of U.S. Ser. No. 09/661,710, filed Sep. 14, 2000.

### FIELD OF THE INVENTION

The present invention is directed to a series of 3-pyrrolidinyl-oxy-3′-pyridyl ether compounds, a method for selectively controlling neurotransmitter release in mammals using these compounds, and pharmaceutical compositions containing these compounds. Preferred compounds are 3-pyrrolidinylmethoxy -3′-(5′- and/or 6′-substituted) pyridyl ethers.

### BACKGROUND OF THE INVENTION

Compounds that selectively control chemical synaptic transmission offer therapeutic utility in treating disorders that are associated with dysfunctions in synaptic transmission. This utility may arise from controlling either pre-synaptic or post-synaptic chemical transmission. The control of synaptic chemical transmission is, in turn, a direct result of a modulation of the excitability of the synaptic membrane. Presynaptic control of membrane excitability results from the direct effect an active compound has upon the organelles and enzymes present in the nerve terminal for synthesizing, storing, and releasing the neurotransmitter, as well as the process for active re-uptake. Postsynaptic control of membrane excitability results from the influence an active compound has upon the cytoplasmic organelles that respond to neurotransmitter action.

An explanation of the processes involved in chemical synaptic transmission will help to illustrate more fully the potential applications of the invention. (For a fuller explanation of chemical synaptic transmission refer to Hoffman et al., “Neuro-transmission: The autonomic and somatic motor nervous systems.” In: Goodman and Gilman's, The Pharmacological Basis of Therapeutics, 9th ed., J. G. Hardman, L. E. Limbird, P. B. Molinoff, R. W. Ruddon, and A. Goodman Gilman, eds., Pergamon Press, New York, 1996, pp. 105-139).

Typically, chemical synaptic transmission begins with a stimulus that depolarizes the transmembrane potential of the synaptic junction above the threshold that elicits an all-or-none action potential in a nerve axon. The action potential propagates to the nerve terminal where ion fluxes activate a mobilization process leading to neurotransmitter secretion and “transmission” to the postsynaptic cell. Those cells which receive communication from the central and peripheral nervous systems in the form of neurotransmitters are referred to as “excitable cells.” Excitable cells are cells such as nerves, smooth muscle cells, cardiac cells and glands. The effect of a neurotransmitter upon an excitable cell may be to cause either an excitatory or an inhibitory postsynaptic potential (EPSP or IPSP, respectively) depending upon the nature of the postsynaptic receptor for the particular neurotransmitter and the extent to which other neurotransmitters are present. Whether a particular neurotransmitter causes excitation or inhibition depends principally on the ionic channels that are opened in the postsynaptic membrane (i.e., in the excitable cell).

EPSPs typically result from a local depolarization of the membrane due to a generalized increased permeability to cations (notably Na⁺ and K⁺), whereas IPSPs are the result of stabilization or hyperpolarization of the membrane excitability due to a increase in permeability to primarily smaller ions (including K⁺ and Cl⁻). For example, the neurotransmitter acetylcholine excites at skeletal muscle junctions by opening permeability channels for Na⁺ and K⁺. At other synapses, such as cardiac cells, acetylcholine can be inhibitory, primarily resulting from an increase in K+ conductance.

The biological effects of the compounds of the present invention result from modulation of a particular subtype of acetylcholine receptor. It is, therefore, important to understand the differences between two receptor subtypes. The two distinct subfamilies of acetylcholine receptors are defined as nicotinic acetylcholine receptors and muscarinic acetylcholine receptors. (See Goodman and Gilman's. The Pharmacological Basis of Therapeutics, op. cit.).

The responses of these receptor subtypes are mediated by two entirely different classes of second messenger systems. When the nicotinic acetylcholine receptor is activated, the response is an increased flux of specific extracellular ions (e.g. Na⁺, K⁺ and Ca⁺⁺) through the neuronal membrane. In contrast, muscarinic acetylcholine receptor activation leads to changes in intracellular systems that contain complex molecules such as G-proteins and inositol phosphates. Thus, the biological consequences of nicotinic acetylcholine receptor activation are distinct from those of muscarinic receptor activation. In an analogous manner, inhibition of nicotinic acetylcholine receptors results in still other biological effects, which are distinct and different from those arising from muscarinic receptor inhibition.

As indicated above, the two principal sites to which drug compounds that affect chemical synaptic transmission may be directed are the presynaptic membrane and the post-synaptic membrane. Actions of drugs directed to the presynaptic site may be mediated through presynaptic receptors that respond to the neurotransmitter which the same secreting structure has released (i.e., through an autoreceptor), or through a presynaptic receptor that responds to another neurotransmitter (i.e., through a heteroreceptor). Actions of drugs directed to the postsynaptic membrane mimic the action of the endogenous neurotransmitter or inhibit the interaction of the endogenous neurotransmitter with a postsynaptic receptor.

Classic examples of drugs that modulate postsynaptic membrane excitability are the neuromuscular blocking agents which interact with nicotinic acetylcholine-gated channel receptors on skeletal muscle, for example, competitive (stabilizing) agents, such as curare, or depolarizing agents, such as succinylcholine.

In the central nervous system, postsynaptic cells can have many neurotransmitters impinging upon them. This makes it difficult to know the precise net balance of chemical synaptic transmission required to control a given cell. Nonetheless, by designing compounds that selectively affect only one pre- or postsynaptic receptor, it is possible to modulate the net balance of all the other inputs. Obviously, the more that is understood about chemical synaptic transmission in CNS disorders, the easier it would be to design drugs to treat such disorders.

Knowing how specific neurotransmitters act in the CNS allows one to predict the disorders that may be treatable with certain CNS-active drugs. For example, dopamine is widely recognized as an important neurotransmitter in the central nervous systems in humans and animals. Many aspects of the pharmacology of dopamine have been reviewed by Roth and Elsworth, “Biochemical Pharmacology of Midbrain Dopamine Neurons”, In: Psychopharmacology: The Fourth Generation of Progress, F. E. Bloom and D. J. Kupfer, Eds., Raven Press, NY, 1995, pp 227-243). Patients with Parkinson's disease have a primary loss of dopamine containing neurons of the nigrostriatal pathway, which results in profound loss of motor control. Therapeutic strategies to replace the dopamine deficiency with dopamine mimetics, as well as administering pharmacologic agents that modify dopamine release and other neurotransmitters have been found to have therapeutic benefit (“Parkinson's Disease”, In: Psychopharmacology: The Fourth Generation of Progress, op. cit., pp 1479-1484).

New and selective neurotransmitter controlling agents are still being sought, in the hope that one or more will be useful in important, but as yet poorly controlled, disease states or behavior models. For example, dementia, such as is seen with Alzheimer's disease or Parkinsonism, remains largely untreatable. Symptoms of chronic alcoholism and nicotine withdrawal involve aspects of the central nervous system, as does the behavioral disorder Attention-Deficit Disorder (ADD). Specific agents for the treatment of these and related disorders are few in number or non-existent.

A more complete discussion of the possible utility as CNS-active agents of compounds with activity as cholinergic ligands selective for neuronal nicotinic receptors, (i.e., for controlling chemical synaptic transmission) may be found in U.S. Pat. No. 5,472,958, to Gunn et al., issued Dec. 5, 1995, which is incorporated herein by reference.

Existing acetylcholine agonists are therapeutically suboptimal in treating the conditions discussed above. For example, such compounds have unfavorable pharmacokinetics (e.g., arecoline and nicotine), poor potency and lack of selectivity (e.g., nicotine), poor CNS penetration (e.g., carbachol) or poor oral bioavailability (e.g., nicotine). In addition, other agents have many unwanted central agonist actions, including hypothermia, hypolocomotion and tremor and peripheral side effects, including miosis, lachrymation, defecation and tachycardia (Benowitz et al., in: Nicotine Psychopharmacology, S. Wonnacott, M. A. H. Russell, & I. P. Stolerman, eds., Oxford University Press, Oxford, 1990, pp. 112-157; and M. Davidson, et al., in Current Research in Alzheimer Therapy, E. Giacobini and R. Becker, ed.; Taylor & Francis: New York, 1988; pp 333-336).

Williams et al. reports the use of cholinergic channel modulators to treat Parkinson's and Alzheimer's Diseases. M. Williams et al., “Beyond the Tobacco Debate: Dissecting Out the Therapeutic Potential of Nicotine”, Exp. Opin. Invest. Drugs 5, pp. 1035-1045 (1996). Salin-Pascual et al. reports short-term improvement of non-smoking patients suffering from depression by treatment with nicotine patches. R. J. Salin-Pascual et al., “Antidepressant Effect of Transdermal Nicotine Patches in Non-Smoking Patients with Major Depression”, J. Clin. Psychiatry, v. 57 pp. 387-389 (1996).

Ethers which are useful as antagonists of specific 5-hydroxy tryptamine (5-HT) receptors are disclosed in GB 2 208 510A; U.S. Pat. Nos. 4,929,625; 5,082,843 and 4,997,839. However, these references disclose a 2-pyridyl moiety linked by oxygen to a saturated azabicyclic ring such as quinuclidyl or tropanyl. Analgesic pyridine-2-ethers are also disclosed in U.S. Pat. Nos. 4,946,836 and 4,643,995. In these references, a 2-pyridyl moiety is linked to a nitrogen-containing cycloaliphatic ring through an —O—(CH₂)ₙ— linkage.

3-Pyridyloxymethyl heterocyclic ether compounds useful in controlling chemical synaptic transmission are disclosed in U.S. Pat. No. 5,629,325; wherein a 3-pyridyl moiety is linked to a nitrogen-containing cycloaliphatic ring through an —O—CH₂— linkage. PCT Patent Application WO 94/08992 discloses various 3-pyridyloxy-heterocyclic compounds that are either unsubstituted or mono-substituted on the pyridine rings with groups such as Br, Cl, F, hydroxyl, C₁-C₃ alkyl or C₁-C₃ alkoxy, such compounds also described as having utility in enhancing cognitive function.

1,3-disubstituted pyrrolidines which have pharmacological action on the central nervous system wherein the pyrrolidine nitrogen is substituted by an—(CH₂)ₙ—B group, and ether-linked to a substituted pyridyl, among others are disclosed in U.S. Pat. No. 5,037,841.

Cyclic amine compounds effective against senile dementia wherein the ring is ether-linked to a substituted 3-pyridyl among others are disclosed in European Patent Application No. 0 673 927 A1.

Aza ring ether derivatives and their use as nicotinic ACH receptor modulators are disclosed in WO 99/24422.

However, there is still a need for improved compounds for controlling chemical synaptic transmission.

It is therefore an object of this invention to provide novel 3-pyrrolidinyl-oxy-3′-pyridyl ether compounds. It is a further object of this invention to provide such compounds which selectively control neurotransmitter release.

### SUMMARY OF THE INVENTION

The present invention is directed to a series of 3-pyrrolidinyloxy-3′-pyridyl ether compounds, a method for selectively controlling neurotransmitter release in mammals using these compounds, and pharmaceutical compositions including these compounds. More particularly, the present invention is directed to compounds of the formula I

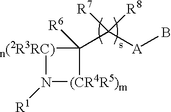

wherein m and n are each integers of from 1 to 6, and the sum of

n+m is from 2 to 7;

s is an integerof 0to 3;

R¹ is selected from the group consisting of hydrogen, lower alkyl, alkenyl, alkynyl, aryl, aralkyl, aryloxy, arylamino, biaryl, thioaryl, aroyl, heterocyclyl, alkylheterocyclyl, heterocycloyl, cyanomethyl, cycloalkyl, cycloalkenyl and cycloalkylalkyl;

R², R³, R⁴, R⁵, R⁶, R⁷ and R⁸ at each occurance, are each independently selected from the group consisting of hydrogen, hydroxyl, amnino, halogen, lower alkyl, lower alkenyl, lower alkynyl, lower alkoxy, alkenoxy, alkynoxy, thioalkoxy, aliphatic acyl, —CF₃, nitro, cyano, —N(C₁-C₃ alkyl)—C(O)(C₁-C₃ alkyl), —C₁—C₃ alkylamino, alkenylamino, alkynylamino, di(C₁-C₃ alkyl)amino, —C(O)O—(C₁-C₃ alkyl), —CH═NOH, —C(O)NH—(C₁-C₃ alkyl), —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, thioaryl, heterocyclyl, heterocycloyl, alkylaryl, aralkyl, alkylheterocyclyl, sulfonyl, sulfonamido, carbamate, aryloxyalkyl, carboxyl and —C(O)NH(benzyl);

A is selected from the group consisting of —C(R²)(R³)—, —O—, —S—, —N(R¹)—, —SO₂N(R¹)—, —C(O)N(R¹)—, —NR¹C(O)—, —C(O)—, —C(O)O—, —OC(O)— and —N(R¹)SO₂—;

B is selected from the group consisting of heteroaryl and heteroaryl alkyl;

and salts thereof;

with the proviso that when s is 0, the sum of m+n is from 2-5, A=—O— and R¹ is hydrogen or methyl, B is not 3-pyridyl, 5-chloro-3-pyridyl or 2-chloro-3-pyridyl;

and with the further proviso that when R², R³, R⁴, R⁵, R⁶, R⁷ or R⁸ are attached to a carbon which is alpha to a heteroatom, R², R³, R⁴, R^{5,} R⁶, R⁷ or R⁸ are not halogen, hydroxyl or amino. Presently preferred compounds have the structure shown below (formula

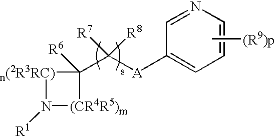

wherein

m and n are each integers of from 1 to 6, and the sum of n+m is from 2to 7;

s is an integer of 0 or 1;

R¹ is selected from the group consisting of hydrogen, lower alkyl, alkenyl, alkynyl, aryl, aralkyl, aryloxy, arylamino, biaryl, thioaryl, aroyl, heterocyclyl, alkylheterocyclyl, heterocycloyl, cyanomethyl, cycloalkyl, cycloalkenyl and cycloalkylalkyl;

R^{2,} R^{3,} R^{4,} R^{5,} R^{6,} R⁷ and R⁸ at each occurence are each independently selected from the group consisting of hydrogen, hydroxyl, amino, halogen, lower alkyl, lower alkenyl, lower alkynyl, lower alkoxy, alkenoxy, alkynoxy, thioalkoxy, aliphatic acyl, —CF₃, nitro, cyano, —N(C₁-C₃ alkyl)—C(O)(C₁-C₃ alkyl), —C₁-C₃ alkylamino, alkenylamino, alkynylamino, di(C₁-C₃ alkyl)amino, —C(O)O—(C₁-C₃ alkyl), —C(O)NH—(C₁-C₃ alkyl), —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, thioaryl, heterocyclyl, heterocycloyl, alkylaryl, aralkyl, alkylheterocyclyl, sulfonyl, sulfonamido, carbamate, aryloxyalkyl, carboxyl and —C(O)NH(benzyl);

R⁹, at each occurrence, is independently selected from the group consisting of halogen, hydroxyl, lower alkyl, lower alkenyl, lower alkynyl, lower alkoxy, alkenoxy, alkynoxy, thioalkoxy, aliphatic acyl, —CF₃, nitro, cyano, —N(C₁-C₃ alkyl)—C(O)(C₁-C₃ alkyl), —C₁-C₃ alkylamino, alkenylamino, alkynylamino, di(C₁-C₃ alkyl)amino, —C(O)O—(C₁-C₃ alkyl), —C(O)NH—(C₁-C₃ alkyl), —CH═NOH, —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, thioaryl, heterocyclyl, heterocycloyl, alkylaryl, aralkyl, alkylheterocyclyl, sulfonyl, sulfonamido, carbamate, aryloxyalkyl, carboxyl, amino and —C(O)NH(benzyl);

A is selected from the group consisting of —C(R²)(R³)—, —O—, —S—, —N(R¹)—, —SO₂N(R¹)—, —C(O)N(R¹)—, —NR¹C(O)—, —C(O)—, —C(O)O—, —OC(O)— and —N(R¹)SO₂—; and

p is an integer of from one to four;

with the proviso that when s=0, A=—O— and p=1, R¹ is hydrogen or methyl, R⁹ is not 5-chloro or 2-chloro;

with the proviso that when A is —O—, s=0, R¹ is hydrogen or methyl and the sum of m+n is from two to five, p is not 0;

and with the further proviso that when R², R³, R⁴, R⁵, R⁶, R⁷ or R⁸ are attached to a carbon which is alpha to a heteroatom, R², R³, R⁴, R⁵, R⁶, R⁷ or R⁸ are not halogen, hydroxyl or amino. Presently preferred compounds also have the structure shown below (formula III)

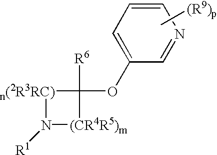

wherein

m and n are each integers of 1 to 4 and the sum of m and n is 5;

is an integer of one to four;

R¹ is selected from the group consisting of hydrogen, lower alkyl, alkenyl, alkynyl, aryl, aralkyl, aryloxy, arylamino, biaryl, thioaryl, aroyl, heterocyclyl, heterocycloyl, alkylheterocyclyl, cyanomethyl, cycloalkyl, cycloalkenyl and cycloalkylalkyl;

R², R³, R⁴ and R⁵ are each independently selected from the group consisting of hydrogen, hydroxyl, amino, halogen, lower alkyl, lower alkenyl, lower alkynyl, lower alkoxy, alkenoxy, alkynoxy, thioalkoxy, aliphatic acyl, —CF₃, nitro, cyano, —N(C₁-C₃ alkyl)—C(O)(C₁-C₃ alkyl), -C₁-C₃ alkylamino, alkenylamino, alkynylamino, di(C₁-C₃ alkyl)amino, —C(O)O—(C₁-C₃ alkyl), —C(O)NH-(C₁-C₃ alkyl), —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, thioaryl, alkylaryl, aralkyl, sulfonyl, heterocyclyl, heterocycloyl, alkylheterocyclyl, sulfonamido, carbamate, aryloxyalkyl, carboxyl and —C(O)NH(benzyl);

R⁶ is selected from the group consisting of hydrogen, lower alkyl, lower alkenyl, lower alkynyl, lower alkoxy, alkenoxy, alkynoxy, thioalkoxy, aliphatic acyl, —CF₃, nitro, cyano, —N(C₁-C₃ alkyl)-C(O)(C₁-C₃ alkyl), —C₁-C₃ alkylamino, alkenylamino, alkynylamino, di(C₁-C₃ alkyl)amino, —C(O)O—(C₁-C₃ alkyl), —C(O)NH—(C₁-C₃ alkyl), —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, thioaryl, heterocyclyl, heterocycloyl, alkylaryl, aralkyl, alkylheterocyclyl, sulfonyl, sulfonamido, carbamate, aryloxyalkyl, carboxyl and —C(O)NH(benzyl);

wherein R⁹, at each occurrence, is independently selected from the group consisting of halogen, hydroxyl, lower alkyl, lower alkenyl, lower alkynyl, lower alkoxy, alkenoxy, alkynoxy, thioalkoxy, aliphatic acyl, —CF₃, nitro, amino, cyano, —N(C₁-C₃ alkyl)—CO(C₁-C₃ alkyl), —C₁-C₃ alkylamino, alkenylamino, alkynylamino, di(C₁-C₃ alkyl)amino, —C(O)O—(C₁-C₃ alkyl), —C(O)NH—(C₁-C₃ alkyl), —C(O)N(C₁-C₃ alkyl)₂, —CH═NOH, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, thioaryl, heterocyclyl, heterocycloyl, alkylaryl, aralkyl, alkylheterocyclyl, sulfonyl, sulfonamido, carbamate, aryloxyalkyl, carboxyl and —C(O)NH(benzyl);

and salts thereof;

with the proviso that when R², R³, R⁴ or R⁵ are attached to a carbon which is alpha to a heteroatom, R², R³, R⁴ or R⁵ are not halogen, hydroxyl or amino. Presently most preferred compounds have the structure shown below (formula IV)

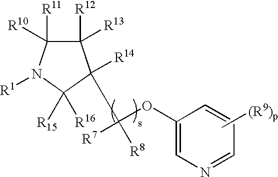

wherein

s is an integer of 0 or 1;

R¹ is selected from the group consisting of hydrogen, lower alkyl, alkenyl, alkynyl, aryl, aralkyl, aryloxy, arylamino, biaryl, thioaryl, aroyl, heterocyclyl, heterocycloyl, alkylheterocyclyl, cyanomethyl, cycloalkyl, cycloalkenyl and cycloalkylalkyl;

R⁷, R⁸, R¹⁰, R¹¹, R¹⁵, and R¹⁶ are independently selected from the group consisting of hydrogen, lower alkyl, lower alkenyl, lower alkynyl, lower alkoxy, alkenoxy, alkynoxy, thioalkoxy, aliphatic acyl, —CF₃, nitro, cyano, —N(C₁-C₃ alkyl)—C(O)(C¹-C₃ alkyl), —C₁-C₃ alkylamino, alkenylamino, alkynylamino, di(C₁-C₃ alkyl)amino, —C(O)O—(C₁-C₃ alkyl), —C(O)NH—(C₁-C₃ alkyl), —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, thioaryl, heterocyclyl, heterocycloyl, alkylaryl, aralkyl, alkylheterocyclyl, sulfonyl, sulfonamido, carbamate, aryloxyalkyl, carboxyl and —C(O)NH(benzyl);

R⁹, at each occurrence, is independently selected from the group consisting of halogen, hydroxyl, lower alkyl, lower alkenyl, lower alkynyl, lower alkoxy, alkenoxy, alkynoxy, thioalkoxy, aliphatic acyl, —CF₃, nitro, amino, cyano, —N(C₁-C₃ alkyl)-CO(C₁-C₃ alkyl), —C₁-C₃ alkylamino, alkenylamino, alkynylamino, di(C₁-C₃ alkyl)amino, —C(O)O—(C₁-C₃ alkyl), —C(O)NH-(C₁-C₃ alkyl), —CH═NOH, —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, thioaryl, heterocyclyl, heterocycloyl, alkylaryl, aralkyl, alkylheterocyclyl, sulfonyl, sulfonamido, carbamate, aryloxyalkyl, carboxyl and —C(O)NH(benzyl);

R¹², R¹³ and R¹⁴ are independently selected from the group consisting of hydrogen, halogen, hydroxyl, lower alkyl, lower alkenyl, lower alkynyl, lower alkoxy, alkenoxy, alkynoxy, thioalkoxy, aliphatic acyl, —CF₃, amino, nitro, cyano, —N(C₁-C₃ alkyl)-C(O)(C₁-C₃ alkyl), —C₁-C₃ alkylamino, alkenylamino, alkynylamino, di(C₁-C₃ alkyl)amino, —C(O)O—(C₁-C₃ alkyl), —C(O)NH—(C₁-C₃ alkyl), —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, thioaryl, heterocyclyl, heterocycloyl, alkylaryl, aralkyl, alkylheterocyclyl, sulfonyl, sulfonamido, carbamate, aryloxyalkyl, carboxyl and —C(O)NH(benzyl);

and p is an integer of from one to four;

with the proviso that when s=0, R¹ is hydrogen or methyl and p=1, R⁹ is not 5-chloro or 2-chloro;

and the further proviso that when s=0, R¹ is hydrogen or methyl, p is not 0.

For compounds of Formula IV, R⁹, at each occurrence, may be pyridylethenyl, dimethylhexadienyl, chlorophenyl, thienyl, phenyl, aminophenyl, pyridyl, pyrimidyl, octynyl, lower alkyl, —F, —Cl or —Br. R¹ may be hydrogen or methyl. For compounds of Formula IV, s is preferably 1.

Presently preferred compounds have m=1, A as oxygen and B=substituted 3-pyridyl group, such as 3-pyrrolidinyloxy-3′-5- and/or 6-substituted pyridyl ethers.

Presently preferred compounds are 3-(3-(S)-pyrrolidinyloxy)-5-methylpyridine, 3-(3-(S)-pyrrolidinyloxy)-5-(2-(4-pyridyl)ethenyl)pyridine, 3-(3-(R)-pyrrolidinyloxy)-5-(2-(4-pyridyl)ethenyl)pyridine, 3-(1-methyl-3-(S)-pyrrolidinyloxy)-5-(2-(4-pyridyl)ethenyl)pyridine, 3-(1-methyl-3-(R)-pyrrolidinyloxy)-5-(2-(4-pyridyl)ethenyl)pyridine 3-(3-(R)-pyrrolidinylmethoxy)-5-methylpyridine, 3-(3-(S)-pyrrolidinyloxy-5-(5,5-dimethylhexadienyl)pyridine, 3-(3-(S)-pyrrolidinyloxy)-5-(1-octynyl)pyridine, 3-(3-(R)-pyrrolidinyloxy)-5-(1-octynyl)pyridine, 3-(1-methyl-3-(R)-pyrrolidinyloxy)-5-(1-octynyl)pyridine, 3-(3-(S)-pyrrolidinyloxy)-5-(5-pyrimidyl)pyridine, 3-(1-methyl-3-(S)-pyrrolidinyloxy)-5-(3-pyridyl)pyridine, 3-(3-(R)-pyrrolidinyloxy)-5-(5-pyrimidinyl)pyridine, 3-(3-(S)-pyrrolidinyloxy)-5-(3-aminophenyl)pyridine, 3-(3-(S)-pyrrolidinyloxy)-5-phenylpyridine, 3-(1-methyl-3-(S)-pyrrolidinyloxy)-5-phenylpyridine, 3-(3-(R)-pyrrolidinyloxy)-5-phenylpyridine, 3-(3-(S)-pyrrolidinyloxy)-5-thienylpyridine, 3-(3-(R)-pyrrolidinyloxy)-5-thienylpyridine, 3-(1-methyl-3-(R)-pyrrolidinyloxy)-5-thienylpyridine, 3-(3-(S)-pyrrolidinyloxy)-5-(4-chlorophenyl)pyridine, 3-(3-(S)-pyrrolidinyloxy)-5-bromo-6-chloropyridine, 3-(1-methyl-3-(S)-pyrrolidinyloxy)-5-bromo-6-chloropyridine, 3-(1-methyl-3-(S)-pyrrolidinyloxy)-5-(2-(4-pyridyl)ethenyl)-6-chloropyridine, 3-bromo-2-chloro-5- (3-pyrrolidinylmethoxy)pyridine, 3-bromo-2-chloro-5-(1-methyl -pyrrolidinylmethoxy) pyridine, 3-methyl-5-(3-(pyrrolidinyl)methoxy)pyridine, 5-phenyl-3-(3-pyrrolidinylmethoxy)pyridine or salts thereof.

### DETAILED DESCRIPTION OF THE INVENTION

### Definitions of Terms

The term “alkyl” as used herein alone or in combination refers to C₁-C₁₂ straight or branched, saturated or unsaturated (alkenyl, alkynyl, allyl) chain radicals derived from saturated hydrocarbons by the removal of one hydrogen atom. Representative examples of alkyl groups include methyl, ethyl, n-propyl, iso-propyl, n-butyl, sec-butyl, iso-butyl, and tert-butyl among others.

The term “alkenyl”, alone or in combination, refers to a straight-chain or branched-chain alkenyl radical containing from 2 to 10 carbon atoms. Examples of such radicals include, but are not limited to, ethenyl, E- and Z-pentenyl, decenyl and the like.

The term “alkynyl”, alone or in combination, refers to a straight or branched chain alkynyl radical containing from 2 to 10 carbon atoms. Examples of such radicals include, but are not limited to ethynyl, propynyl, propargyl, butynyl, hexynyl, decynyl and the like.

When the term “lower” modifies “alkyl”, “alkenyl”, “alkynyl”, or “alkoxy” it refers to C₁-C₆ substituents.

The term “cycloalkyl” as used herein alone or in combination refers to an aliphatic ring system having 3 to 10 carbon atoms and 1 to 3 rings, including, but not limited to cyclopropyl, cyclopentyl, cyclohexyl, norbornyl, and adamantyl among others. Cycloalkyl groups can be unsubstituted or substituted with one, two or three substituents independently selected from lower alkyl, haloalkyl, alkoxy, thioalkoxy, amino, alkylamino, dialkylamino, hydroxy, halo, mercapto, nitro, carboxaldehyde, carboxy, alkoxycarbonyl and carboxamide. This term is meant to encompass cycloalkenyl and cycloalkynyl groups.

The term “cycloalkenyl” as used herein alone or in combination refers to a cyclic carbocycle containing from 4 to 8 carbon atoms and one or more double bonds. Examples of such cycloalkenyl radicals include, but are not limited to, cyclopentenyl, cyclohexenyl, cyclopentadienyl and the like.

The term “cycloalkylalkyl” as used herein refers to a cycloalkyl group appended to a lower alkyl radical, including, but not limited to cyclohexyl methyl.

The term “halo” or “halogen” as used herein refers to I, Br, Cl or F.

The term “haloalkyl” as used herein refers to a lower alkyl radical, to which is appended at least one halogen substituent, for example chloromethyl, fluoroethyl, trifluoromethyl and pentafluoroethyl among others.

The term “alkoxy”, alone or in combination, refers to an alkyl ether radical, wherein the term “alkyl” is as defined above. Examples of suitable alkyl ether radicals include, but are not limited to, methoxy, ethoxy, n-propoxy, iso-propoxy, n-butoxy, iso-butoxy, sec-butoxy, tert-butoxy and the like.

The term “alkenoxy”, alone or in combination, refers to a radical of formula alkenyl-O-, provided that the radical is not an enol ether, wherein the term “alkenyl” is as defined above. Examples of suitable alkenoxy radicals include, but are not limited to, allyloxy, E- and Z- 3-methyl-2-propenoxy and the like.

The term “alkynoxy”, alone or in combination, refers to a radical of formula alkynyl-O—, provided that the radical is not an -ynol ether. Examples of suitable alkynoxy radicals include, but are not limited to, propargyloxy, 2-butynyloxy and the like.

The term “thioalkoxy”, refers to a thioether radical of formula alkyl-S—, wherein “alkyl” is as defined above.

The term “carboxyl” as used herein refers to —CO₂H.

The term “carboxaldehyde” as used herein refers to —C(O)H wherein R is hydrogen.

The term “carboxamide” as used herein refers to —C(O)NH₂.

The term “alkoxyalkoxy” as used herein refers to R_{b}O—R_{c}O— wherein R_{b} is lower alkyl as defined above and R_{c} is alkylene wherein alkylene is —(CH₂)_{n′}— wherein n′ is an integer from 1 to 6. Representative examples of alkoxyalkoxy groups include methoxymethoxy, ethoxymethoxy, and t-butoxymethoxy among others.

The term “alkylamino” as used herein refers to R_{d}NH— wherein R_{d} is a lower alkyl group, for example, ethylamino, butylamino, among others.

The term “alkenylamino” alone or in combination, refers to a radical of formula alkenyl-NH— - or (alkenyl)₂N—, wherein the term “alkenyl” is as defined above, provided that the radical is not an enamine. An example of such alkenylamino radicals is the allylamino radical.

The term “alkynylamino”, alone or in combination, refers to a radical of formula alkynyl-NH— - or (alkynyl)₂N- wherein the term “alkynyl” is as defined above, provided that the radical is not an enamine. An example of such alkynylamino radicals is the propargyl amino radical.

The term “dialkylamino” as used herein refers to RₑR_{f}N— wherein Rₑ and R_{f} are independently selected from lower alkyl, for example diethylamino, and methyl propylamino, among others.

The term “amino” as used herein refers to H₂N—.

The term “alkoxycarbonyl” as used herein refers to an alkoxyl group as previously defined appended to the parent molecular moiety through a carbonyl group. Examples of alkoxycarbonyl include methoxycarbonyl, ethoxycarbonyl, and isopropoxycarbonyl among others.

The term “heteroaryl” as used herein alone or in combination refers to a group such as furyl, thienyl, pyridyl, pyrrolyl, oxazolyl, thiazolyl, imidazolyl, pyrazolyl, 2-pyrazolinyl, pyrazolidinyl, isoxazolyl, isothiazolyl, 1,2,3-oxadiazolyl, 1,2,3-triazolyl, 1,3,4-thiadiazolyl, pyridazinyl, pyrimidinyl, pyrazinyl, 1,3,5-triazinyl, 1,3,5-trithianyl, indolizinyl, indolyl, isoindolyl, 3H-indolyl, indolinyl, benzo[b]furanyl, 2,3-dihydrobenzofuranyl, benzo[b]thiophenyl, 1H-indazolyl, benzimidazolyl, benzthiazolyl, purinyl, 4H-quinolizinyl, isoquinolinyl, cinnolinyl, phthalazinyl, quinazolinyl, quinoxalinyl, 1,8-naphthridinyl, pteridinyl, carbazolyl, acridinyl, phenazinyl, phenothiazinyl, phenoxyazinyl, or pyrazolo[1,5-c]triazinyl among others.

The term “aryl” or “aromatic” as used herein alone or in combination refers to a carbocyclic aromatic group having about 6 to 12 carbon atoms such as phenyl, naphthyl, indenyl, indanyl, azulenyl, fluorenyl and anthracenyl.

“Arylalkyl” and “alkylaryl” employ the term “alkyl” as defined above.

The term “aralkyl”, alone or in combination, refers to an aryl substituted alkyl radical, wherein the terms “alkyl” and “aryl” are as defined above. Examples of suitable aralkyl radicals include, but are not limited to, phenylmethyl, phenethyl, phenylhexyl, diphenylmethyl, pyridylmethyl, tetrazolyl methyl, furylmethyl, imidazolyl methyl, indolylmethyl, thienylpropyl and the like.

The term “heteroaryl alkyl”, alone or in combination, refers to a heteroaryl substituted alkyl radical, wherein the terms “alkyl” and “heteroaryl” are as defined above.

The term “arylamino”, alone or in combination, refers to a radical of formula aryl-NH— , wherein “aryl” is as defined above. Examples of arylamino radicals include, but are not limited to, phenylamino(anilido), naphthlamino, 2-, 3-, and 4- pyridylamino and the like.

The term “biaryl”, alone or in combination, refers to a radical of formula aryl-aryl, wherein the term “aryl” is as defined above.

The term “thioaryl”, alone or in combination, refers to a radical of formula aryl-S—, wherein the term “aryl” is as defined above. An example of a thioaryl radical is the thiophenyl radical.

The term “aroyl”, alone or in combination, refers to a radical of formula aryl-CO—, wherein the term “aryl” is as defined above. Examples of suitable aromatic acyl radicals include, but are not limited to, benzoyl, 4-halobenzoyl, 4-carboxybenzoyl, naphthoyl, pyridylcarbonyl and the like.

The term “heterocyclyl”, alone or in combination, refers to a non-aromatic 3- to 10-membered ring containing at least one endocyclic N, O or S atom. The heterocycle may be optionally aryl-fused. The heterocycle may also optionally be substituted with at least one substituent which is independently selected from the group consisting of hydrogen, halogen, hydroxyl, amino, nitro, trifluoromethyl, trifluoromethoxy, alkyl, aralkyl, alkenyl, alkynyl, aryl, cyano, carboxy, carboalkoxy, carboxyalkyl, oxo, arylsulfonyl and aralkylaminocarbonyl among others.

The term “heterocycloyl”, alone or in combination, refers to a radical of formula heterocyclyl-CO—, wherein the term “heterocyclyl” is as defined above.

Use of the terms “cycloalkyl”, “heterocyclyl”, “heteroaryl”, “aryl”, “alkenyl”, “alkynyl” or “alkyl” is meant to encompass substituted and unsubstituted moieties. Substitution may be by one or more groups such as alcohols, hydroxyl, nitro, cyano, carboxy, amines, heteroatoms, lower alkyl, lower alkoxy, lower alkoxycarbonyl, acyloxy, halogens, trifluoromethoxy, trifluoromethyl or any of the substituents of the preceding paragraph or any combination of aryl, alkyl, cycloalkyl or heterocyclic groups either attached directly or by suitable linkers. The linkers are typically short chains of 1-3 atoms containing any combination of —C—, —C(O)—, —NH—, —S—, —S(O)—or —S(O)O—. Rings may be substituted multiple times.

The term “mammals” includes humans and other animals.

The term “heteroatom” as used herein encompasses nitrogen, sulfur and oxygen.

The term “alpha” as used herein indicates the position immediately adjacent to the position described.

### Abbreviations

Abbreviations which have been used in the reaction schemes and the examples that follow have the following meanings: BOC for t-butyloxycarbonyl, Et₂O for diethyl ether, EtOAc for ethyl acetate, MeOH for methanol, EDC for ethylene dichloride, DMF for dimethylformamide, LAH for lithium aluminum hydride, DEAD for diethylazodicarboxylate and TFA for trifluoroacetic acid.

The compounds of the present invention can be used in the form of pharmaceutically acceptable salts derived from inorganic or organic acids. The phrase “pharmaceutically acceptable salt” means those salts which are, within the scope of sound medical judgement, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, v. 66, p. 1 et seq. The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quatemized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Preferred salts of the compounds of the invention include phosphate, tris and acetate.

Dosage forms for topical administration of a compound of this invention include powders, sprays, ointments and inhalants. The active compound is mixed under sterile conditions with a pharmaceutically acceptable carrier and any needed preservatives, buffers or propellants which can be required. Opthalmic formulations, eye ointments, powders and solutions are also contemplated as being within the scope of this invention.

Actual dosage levels of active ingredients in the pharmaceutical compositions of this invention can be varied so as to obtain an amount of the active compound(s) which is effective to achieve the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved.

When used in the above or other treatments, a therapeutically effective amount of one of the compounds of the present invention can be employed in pure form or, where such forms exist, in pharmaceutically acceptable salt, ester or prodrug form. Alternatively, the compound can be administered as a pharmaceutical composition containing the compound of interest in combination with one or more pharmaceutically acceptable excipients. The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the present invention will be decided by the attending physician within the scope of sound medical judgement. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than required to achieve the desired therapeutic effect and to gradually increase the dosage until the desired effect is achieved.

The total daily dose of the compounds of this invention administered to a human or lower animal may range from about 0.0001 to about 1000 mg/kg/day. For purposes of oral administration, more preferable doses can be in the range of from about 0.001 to about 5 mg/kg/day. If desired, the effective daily dose can be divided into multiple doses for purposes of administration; consequently, single dose compositions may contain such amounts or submultiples thereof to make up the daily dose.

The present invention also provides pharmaceutical compositions that comprise compounds of the present invention formulated together with one or more non-toxic pharmaceutically acceptable carriers. The pharmaceutical compositions can be specially formulated for oral administration in solid or liquid form, for parenteral injection or for rectal administration.

The pharmaceutical compositions of this invention can be administered to humans and other mammals orally, rectally, parenterally , intracistemally, intravaginally, intraperitoneally, topically (as by powders, ointments or drops), bucally or as an oral or nasal spray. The term “parenterally,” as used herein, refers to modes of administration which include intravenous, intramuscular, intraperitoneal, intrastemal, subcutaneous and intraarticular injection and infusion.

Pharmaceutical compositions of this invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), vegetable oils (such as olive oil), injectable organic esters (such as ethyl oleate) and suitable mixtures thereof. Proper fluidity can be maintained, for example, by the use of coating materials such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid and the like. It may also be desirable to include isotonic agents such as sugars, sodium chloride and the like. Prolonged absorption of the injectable pharmaceutical form can be brought about by the inclusion of agents which delay absorption such as aluminum monostearate and gelatin.

In some cases, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, tragacanth and mixtures thereof.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together.

Methods to form liposomes are known in the art. See, for example, Prescott, Ed., Methods in Cell Biology, Volume XIV, Academic Press, New York, N.Y. (1976), p. 33 et seq.

The term “pharmaceutically acceptable prodrugs” as used herein represents those prodrugs of the compounds of the present invention which are, within the scope of sound medical judgement, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the compounds of the invention. Prodrugs of the present invention may be rapidly transformed in vivo to the parent compound of the above formula, for example, by hydrolysis in blood. A thorough discussion is provided in T. Higuchi and V. Stella, “Pro-drugs as Novel Delivery Systems”, V. 14 of the A.C.S. Symposium Series, and in Edward B. Roche, ed., Bioreversible Carriers in Drug Design, American Pharmaceutical Association and Pergamon Press (1987), hereby incorporated by reference.

The present invention contemplates both synthetic compounds of formulae I-IV of the present invention, as well as compounds formed by in vivo conversion to compounds of the present invention.

Compounds of the present invention may exist as stereoisomers wherein asymmetric or chiral centers are present. These stereoisomers are “R” or “S” depending on the configuration of substituents around the chiral carbon atom. The present invention contemplates various stereoisomers and mixtures thereof. Stereoisomers include enantiomers and diastereomers, and mixtures of enantiomers or diastereomers. Individual stereoisomers of compounds of the present invention may be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic columns.

The compounds of the invention can exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

The present compounds may have activity against disorders which are mediated through the central nervous system. The following references describe various disorders affected by nicotinic acetylcholine receptors: 1) Williams, M.; Arneric, S. P.; “Beyond the Tobacco Debate: Dissecting out the therapeutic potential of nicotine” Exp. Opin. Invest. Drugs, (1996) 5(8), pp.1035-1 045; 2) Arneric, S. P.; Sullivan, J. P.; Williams, W.; “Neuronal nicotinic acetylcholine receptors, Novel targets for central nervous system theraputics”, in Psychopharmacology: The Fourth Generation of Progress, Bloom F E, Kupfer D J (Eds.), Raven Press, New York (1995): 95-109; 3) Arneric, S. P.; Holladay, M. W.; Sullivan, J. P.; “Cholinergic channel modulators as a novel therapeutic strategy for Alzheimer's disease”, Exp. Opin. Invest. Drugs (1996) 5(1): 79-100; 4) Lindstrom, J.; “Nicotinic Acetylchloline Receptors in Health and Disease”, Molecular Neurobiology (1997) 15: 193-222; and 5) Lloyd, G K; Menzaghi, F; Bontempi B; Suto, C; Siegel, R; Akong, M; Stauderman, K; Velicelebi, G; Johnson, E; Harpold, M M; Rao, T S; Sacaan, A I; Chavez-Noriega, L E; Washburn, M S; Vernier, J M; Cosford, N D P; McDonald, L A; “The potential of subtype-selective neuronal nicotinic acetylcholine receptor agonists as therapeutic agents”, Life Sciences (1998) 62 (17/18): 1601-1606. These disorders include, but are not limited to the following: pain (references 1 and 2), Alzheimer's disease (references 1-5), Parkinson's disease (references 1, 4 and 5), memory disfunction, Tourette's syndrome (references 1, 2 and 4), sleep disorders (reference 1), attention deficit hyperactivity disorder (references I and 3), neurodegeneration, inflammation, neuroprotection (references 2 and 3), amyotrophic lateral sclerosis, anxiety (references 1, 2 and 3), depression (reference 2), mania, schizophrenia (references 1, 2 and 4), anorexia and other eating disorders, AIDS-induced dementia, epilepsy (references 1,2 and 4), urinary incontinence (reference 1), Crohn's disease, migraines, PMS, erectile disjunction, substance abuse, smoking cessation (references 1 and 2) and inflammatory bowel syndrome (references 1 and 4) among others.

The compounds and processes of the present invention will be better understood in connection with the following synthetic schemes which illustrate the methods by which the compounds of the invention may be prepared.

The compounds of the present invention may be prepared by the methods outlined below, and illustrated in the accompanying synthetic schemes. For example, nucleophilic substitution of 3,5-dibromopyridine by a suitably N-protected 3-hydroxypyrrolidine leads to the 5-bromo-3-pyridyl ether 1. The remaining halogen may be further substituted using one of several palladium-mediated transformations, such as those indicated in processes A through D (Scheme 1). In addition, it will be recognized by one skilled in the art that other reactions involving the bromine atom are possible, for example, transmetalation with an alkyl lithium or other organometallic reagent, followed by addition of an electrophile including, but not limited to, a carbonyl compound or nitrile, an alkyl halide, or a silyl halide. Moreover, the products of such a metalation operation may be further manipulated by standard synthetic methods to provide for the variety of compounds of the present invention.

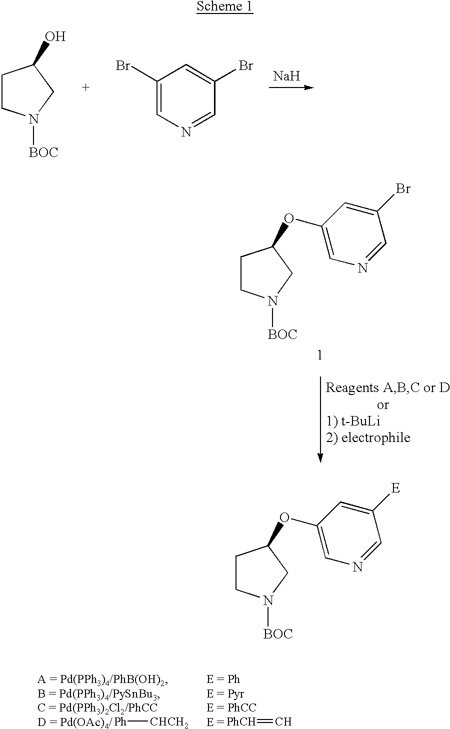

Deprotection of the ring nitrogen can be accomplished, as in Scheme 2, to afford the NH analogs, or alternatively with simultaneous methylation by the Eschweiler-Clarke procedure to produce the N-methyl compounds. Standard methodologies, including reductive amination, convert the NH compounds to other N-alkyl derivatives.

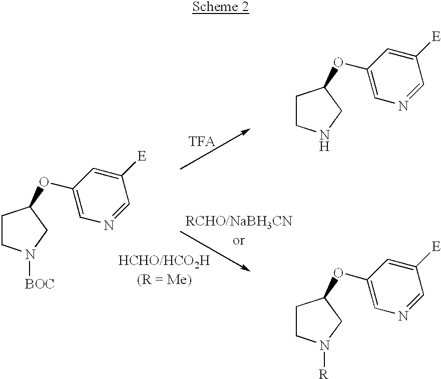

The use of resolved (S) or (R) 3-hydroxypyrrolidine provides the respective chiral (S) or (R) pyridyl ethers. Likewise, extension of the method to other ring sizes can be accomplished by substitution of the appropriate hydroxyazacycle for 3-hydroxypyrrolidine, for example 3-hydroxyazetidine (Rosenberg et al., J. Med. Chem., 1993, 36, 460-467); 3-hydroxypiperidine (Bernet et al., Carbohydr. Res., 1990, 204, 11-25); 4-hydroxypiperidine (Wells et al., Tetrahedron Lett. 1996, 37, 643 9-6442); 4-hydroxyperhydroazepine (Morosawa, Bull. Chem. Soc. Jpn. 1958, 31, 418); and 4-hydroxyperhydro-azocane (Leonard et al., J. Am. Chem. Soc. 1958,80, 4858).

An alternative mode of reaction is outlined in Scheme 3. In this case, a hydroxyl group is converted to a good leaving group, for example a halide or an alkyl or aryl sulfonate ester, and subjected to displacement by a substituted 3-hydroxypyridine. One skilled in the art will understand that such substitutions may be accomplished in other ways including the Mitsunobu modification using activation by a diazodicarboxylate and a phosphine.

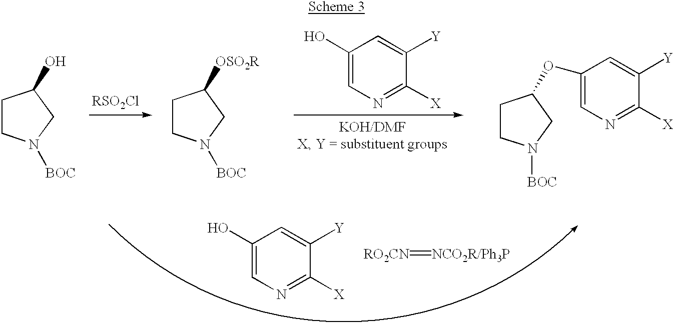

Another preparation of these compounds is illustrated in Scheme 4, where N-benzylpiperidine-2-methanol is prepared and converted to its chloride derivative. Under the basic conditions used for ether formation with a hydroxypyridine, a ring expansion occurs so that a major product is the perhydroazepine 2.

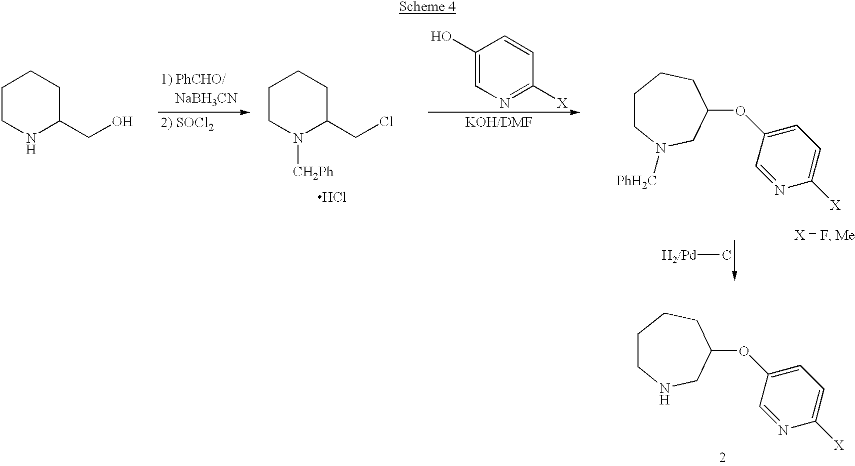

Compounds with an alkylene spacer between the ring and the ether link may be prepared as illustrated in Scheme 5. Nucleophilic substitution with cyanide provides the nitrile 3, which can be converted to the alcohol via hydrolysis and reduction. Alternatively, it will be appreciated by one skilled in the art that addition of an organometallic reagent to the nitrile, followed by hydrolysis will lead to a ketone, which may be reduced or subjected to a second organometallic addition, so that one or two substituents can be introduced at this position.

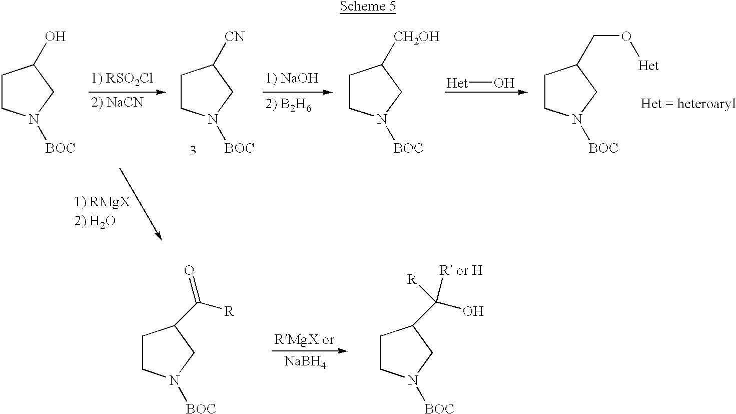

The following examples are presented to describe the preferred embodiments and utilities of the invention, and are not meant to limit the invention unless otherwise stated in the claims appended hereto.

### EXAMPLE 1

3-(3-(S)-pyrrolidinyloxy)-5-methyl pyridine hydrochloride was prepared in the following manner.

1a. 3-(1-Benzyl-3-(S)-pyrrolidinyloxy)-5-bromopyridine was prepared as follows. (S)-(−)-1-Benzyl-3-pyrrolidinol (10 g, 56.4 mmol) was added to a suspension of NaH in DMF at room temperature. After stirring for 1/2 hour, 3,5-dibromopyridine (20 g, 84.6 mmol) was added. The mixture was stirred at 50° C. for 2 hours. The resultant mixture was washed with brine/H₂O (1: 1) in EtOAc. The organic layer was dried, concentrated and chromatographed (silica gel; hexane:EtOAc, 5:1 to 0:1) to afford an oil (7.12 g, 38%). MS (DCl/NH₃): m/z 334 (M+H)⁺. ¹H NMR (CDCl₃, 300 MHz) δ2.00 (m, 1H), 2.35 (m, 1H), 2.58 (m, 1H), 2,74-2.88 (m, 2H), 2.96 (m, 1H), 3.60-3.78 (m, 2H), 4.80 (m, 1H), 7.25-7.38 (m, 6H), 8.17 (d, J=3.0 Hz, 1H), 8.25 (d, J=2.0 Hz, 1H).

1b. 3-(1-Benzyl-3-(S)-pyrrolidinyloxy)-5-methylpyridine was prepared as follows. The compound formed in step 1a (0.55 g, 1.65 mmol) was mixed with MeMgBr (3.0 M, 1.1 mL) and [1,3-bis(diphenylphosphino)propane] nickle(II) chloride (5 mg) in THF (10 mL). The mixture was stirred at 60° C. for 16 hours. The reaction was quenched with H₂O. Solvent was evaporated. The aqueous layer was extracted with CH₂Cl₂ three times. The organic layer was dried (MgSO₄), concentrated and chromatographed (silica gel; CH₂Cl₂: MeOH, 10:0.5) to afford an oil (0.25 g, 56%). MS (DCl/NH₃): m/z 255 (M+H)⁺. ¹H NMR (CDCl₃, 300 MHz) δ2.0 (m, 1H), 2.28 (s, 3H), 2.35 (m, 1H), 2.62 (m, 1H), 2.70-2.82 (m, 2H), 3.00 (m, 1H), 3.64-3.78 (m, 2H), 4.82 (m, 1H), 6.96 (m, 1H), 7.30 (m, 1H), 7.30-7.38 (m, 4H), 8.02-8.04 (m, 2H).

1c. 3-Methyl-5-(3-(S)-pyrrolidinyloxy) pyridine was prepared as follows.

The compound from step 1b (0.25 g, 0.93 mmol) in EtOAc was hydrogenated in the presence of Pd-C (0.lg, 10%) for 3 days. After filtration, solvent was evaporated. The product was obtained (77mg, 46%) as an oil. MS (DCl/NH₃): m/z 179 (M+H)⁺. The free base was converted to HCl salt in ether to afford the title product as a light yellow oil. MS (DCl/NH₃): m/z 179. ¹H NMR (D₂O, 300 MHz) δ2.36-2.44 (m, 2H), 2.42 (s, 3H), 3.50-3.70 (m, 5H), 5.38 (m, 1H), 7.64 (s, 1H), 8.20 (br, s, 2H). Calc'd. Anal. for C₁₀H₁₄N₂O.1.3 HCl.H₂O: C, 49.50; H, 7.14; N, 11.52. Found: C, 49.90; H, 7.24; N, 11.12.

### EXAMPLE 2

3-(3-(S)-pyrrolidinyloxy) pyridine hydrochloride was prepared in the following manner.

2a. 3-(1-BOC-3-(S)-pyrrolidinyloxy)-5-bromopyridine was first made as follows. 1-Chloroethyl chloroformate (3.0 mL, 27.6 mmol) was added to a solution of 3-(1-N-benzyl-3-(S)-pyrrolidinyloxy)-5-bromopyridine (3.8 g, 11.4 mmol) in 1,2-dichloroethane (50 mL) at 0 ° C. The mixture was then stirred and refluxed for 3 hours. Solvent was evaporated and a dark brown oil was obtained. MeOH (20 mL) was added to the residue. The resultant mixture was stirred under reflux for 1 hour. Solvent was evaporated. The resultant residue was treated with di-tert-butyl dicarbonate (12.7 g, 6.0 mmol) and triethylamine (12.0 mL) in CH₂Cl₂ (30 mL), then stirred at room temperature for 16 hours. Solvent was evaporated. The residue was chromatographed (silica gel; hexane:EtOAc, 5:1 to 2:1) to afford a yellow solid (2.5 g, 65%). MS (DCl/NH₃): m/z 345 (M+H)⁺. ¹H NMR (CDCl₃, 300 MHz) δ1.48 (s, 9H), 2.14-2.22 (m, 2H), 3.44-3.70 (m, 4H), 4.96 (m, 1H).7.36 (m, 1H), 9.20 (m, 1H), 8.32 (s, 1H).

2b. 3-(3-(S)-pyrrolidinyloxy)-5-(2-(4-pyridyl)ethenyl)pyridine hydrochloride

b 3-(1-BOC-3-(S)-pyrrolidinyloxy)-5-bromopyridine (0.22 g, 0.63 mmol) was dissolved in acetonitrile (10 mL). 4-Vinylpyridine (0.14 mL, 1.3 mmol), palladium (II) acetate (20 mg), tri-o-tolylphosphine (0.1 g) and triethylamine (0.2 mL) were added. The mixture was stirred and refluxed for 16 hours. Solvent was then evaporated, and the residue was chromatographed (silica gel; CH₂Cl₂: MeOH, 10:0.2 to 10:0.5) to afford an oil (0.18 g, 78%). MS (DCl/NH₃): m/z 368 (M+H)⁺. ¹H NMR (CDCl₃, 300 MHz) δ1.48 (s, 9H), 2.10-2.30 (m, 2H), 3.48-3.70 (m, 3H), 3.70 (m, 1H), 5.00 (m, 1H), 7.07(d, J=16.2 Hz, 1H), 7.27 (d, J=16.5 Hz, 1H), 7.33 (s, 2H), 7.40 (d, J=5.5 Hz, 2H), 8.24 (s, 1H), 8.40 (s, 1H), 8.62 (s, 1H). The product was converted to salt with 4.0 M HCl in 1,4-dioxane as a yellow hygroscopic solid: mp 96° C. (dec). MS (DCl/NH₃): m/z 268 (M+H)⁺. ¹H NMR (D₂O, 300 MHz) δ2.38-2.46 (m, 2H), 3.50-3.72 (m, 4H), 5.42 (m, 1H), 7.50 (d, J=16.3 Hz, 1H), 7.80 (d, J=16.2 Hz, 1H), 7.85 (br, s, 1H), 8.13 (d, J=6.8 Hz, 2H), 8.33 (s, 1H), 8.53 (s, 1H), 8.68 (d, J=6.8 Hz, 2H). Calc'd. Anal. For C₁₆H₁₇N₃O.3.6 HCl.H₂O: C, 46.13; H, 5.47; N, 10.06. Found: C, 46.52; H, 5.57; N, 9.66.

### EXAMPLE 3

3-(3-(R)-Pyrrolidinyloxy)-5-(2-(4-pyridyl)ethenyl)pyridine hydrochloride was prepared according to the procedure as described in Example 2, except 3-(l-N-BOC-3-(S)-pyrrolidinyloxy)-5-bromopyridine was replaced with 3-(1-N-BOC-3-(R)-pyrrolidinyloxy)-5-bromopyridine. 3-(3-(R)-pyrrolidinyloxy)-5-(2-(4-pyridyl)ethenyl)pyridine hydrochloride was obtained as a light yellow solid: mp 200° C. (dec). MS (DCl/NH₃): m/z 268. ¹H NMR (D₂O, 300 MHz) δ2.40-2.50 (m, 2H), 3.54-3.62 (m, 2H), 3.62-3.80 (m, 3H), 5.50 (m, 1H), 7.60 (d, J=16.5 Hz, 1H), 7.84 (d, J=16.0 Hz, 1H), 8.18-8.22 (m, 3H), 8.48 (d, J=2.5 Hz, 1H), 8.66 (s, 1H), 8.74 (d, J=3.5 Hz, 2H). (M+H)⁺. Calc'd. Anal. For C₁₆H₁₇N₃O₄.HCl.0.55 H₂O: C, 45.42; H, 5.27; N, 9.93. Found: C, 45.74; H, 5.55; N, 9.54. [α]_{D}²³ −12.9 (c 0.6, MeOH).

### EXAMPLE 4

3-(1-Methyl-3-(S)-pyrrolidinyloxy)-5-(2-(4-pyridyl)ethenyl)pyridine hydrochloride was prepared in the following manner.

3-(3-(S)-pyrrolidinyloxy)-5-(2-(4-pyridyl)ethenyl)pyridine hydrochloride from Example 2 (0.14 g, 0.52 mmol) was dissolved in acetic acid (0.2 mL). Formaldehyde (70%, 0.15 mL), H₂0 (5 mL), and NaCNBH₃(0.1 g) were added and the pH of the solution was adjusted to 5. The mixture was stirred at room temperature for 16 hours, basified, and the desired product was then extracted with CH₂Cl₂ three times. The organic layer was dried (MgSO₄), filtered, concentrated and chromatographed (silica gel; CH₂Cl₂ : MeOH, 10 :1) to afford an oil (35 mg, 24%). MS (DCl/NH₃): m/z 282 (M+H)⁺. The free base was converted to salt with 1.0 M HCl in ether to afford the title compound as a hygroscopic oil. MS (DCl/NH₃):m/z282(M+H)⁺. ¹H NMR(D₂O, 300MHz) δ2.40 (m, 1H), 2.70 (m, 1H), 3.08 (s, 3H), 3.40 (m, 1H), 3.80-4.02 (m, 2H), 5.38-5.44 (m, 2H), 7.42 (d, J=16.3 Hz, 1H), 7.70 (d, J=16.2 Hz, 1H), 7.78 (s, 1H), 7.96 (d, J=6.8 Hz, 2H), 8.28 (s, 1H), 8.46 (s, 1H), 8.62 (d, J=6.8 Hz, 2Calc'd. Anal. For C₁₃H₁₉N₃O.03.4 HCl.0.3 H₂O: C, 49.65; H, 5.64; N, 10.22. Found: C, 49.25; H, 5.73; N, 10.62. [α]_{D}²³ −5.2 (c 1.1, MeOH)

### EXAMPLE 5

3-(1-Methyl-3-(R)-pyrrolidinyloxy)-5-(2-(4-pyridyl)ethenyl)pyridine hydrochloride was prepared by the same procedure as described in Example 4, except the 3-(3-(S)-pyrrolidinyloxy)-5-(2-(4-pyridyl)ethenyl)pyridine hydrochloride was replaced with the 3-(3-(R)-pyrrolidinyloxyl)-5-(2-(4-pyridyl)ethenyl)pyridine hydrochloride. A light yellow hygroscopic solid was obtained: mp 88-90° C. ¹H NMR (D₂O, 300 MHz) δ2.30-2.45 (brs, 1H), 2.60-2.80 (brs, 1H), 3.06 (s, 3H), 3.30-4.00 (m, 4H), 5.40 (m, 1H), 7.37 (d, J=17.0 Hz, 1H), 7.65 (d, J=16.5 Hz, 1H), 7.70 (s 1H), 7.91 (d, J=5.5 Hz, 2H), 8.24 (s, 1H), 8.44 (s, 1H), 8.60 (dd J=5.5 Hz, 2H). Calc'd. Anal. For C₁₇H₁₉N₃O.2.1 HCl.1.2 H₂O: C, 53.80; H, 6.24; N, 11.06. Found: C, 54.17; H, 6.29; N, 10.66. [α]_{D}²³ −13.6 (c 0.6, MeOH).

### EXAMPLE 6

3-(3-(R)-pyrrolidinyloxy)-5-methylpyridine hydrochloride was prepared as follows.

6a. First, 3-(1-BOC-3-(R)-pyrrolidinyloxyl)-5-methylpyridine was synthesized. 3-(1-BOC-3-(R)-pyrrolidinyloxyl)-5-bromopyridine (prepared as in Example 2a, 0.30 g, 0.88 mmol) was reacted with MeMgBr (3.0 M, 0.73 mL), [1,3-bis(diphenylphosphino)propane]nickle(II) chloride (3 mg) in THF (10 mL) according to the procedure of Example 1b. The residue was then purified on column to give an oil (75 mg, 31%). MS (DCl/NH₃): m/z 279 (M+H)⁺.

6b. Next, 3-(3-(R)-pyrrolidinyloxy)-5-methylpyridine hydrochloride was prepared by converting the compound obtained in step 6a to the HCl salt with 4.0 M HCl in 1,4-dioxane. A yellow oil was obtained. ¹H NMR (D₂O, 300 MHz) δ2.34-2.45 (m, 2H), 2.42 (s, 3H), 3.52-3.68 (m, 5H), 5.40 (m, 1H), 7.70(s, 1H), 8.25 (br, s, 2H). Calc'd.Anal. for C₁₀H₁₄N₂O.3.2 HCl.H₂O: C, 40.73; H, 5.88; N, 9.50. Found: C, 40.91; H, 5.87; N, 9.18.

### EXAMPLE 7

3-(3-(S)-Pyrrolidinyloxy-5-(5,5-dimethylhexadienyl)pyridine hydrochloride was synthesized in the following manner. 3-(1-BOC-3-(S)-pyrrolidinyloxyl)-5-bromopyridine from Example 2a (0.21 g, 0.63 mmol) was dissolved in acetonitrile (10 mL) in a sealed tube. 5,5-Dimethylhexadiene (0.25 g , 2.3 mmol), palladium (II) acetate (17 mg), tri-o-tolylphosphine (85 mg) and triethylamine (1.5 mL) were added. The reaction mixture was stirred and heated at 95° C. for 48 hours. Work-up according to the procedure described in Example 2b gave the free base as an oil (0.16 g, 68%). The compound was converted to HCl salt with 4.0 M HCl in 1,4-dioxane as a light yellow solid: mp 148° C. (dec). MS (DCl/NH₃):m/z 273 (M+H)⁺. ¹H NMR (D₂O, 300 MHz) δ1.08 (s, 9H), 2.36-2.46 (m, 2H), 3.45-3.62 (m, 2H), 3.64-3.78 (m, 2H), 5.42 (m, 1H), 6.20 (d, J=15.6, 1H), 6.34 (dd, J=10, 15.6 Hz, 1H), 6.58 (d, J=15.6 Hz, 1H), 7.11 (dd, J=10, 15.8 Hz, 1H), 7.98 (s, 1H), 8.25 (s, 1H), 8.39 (s, 1H). Calc. Anal. For C₁₇H₂₄N₂O.2.5 HCl.0.3 H₂O: C, 55.34; H, 7.40; N, 7.59. Found: C, 55.61; H, 7.50; N, 7.22. [α]_{D}²³ 20.9 (c 0.6, MeOH).

### EXAMPLE 8

3-(3-(S)-pyrrolidinyloxy)-5-(1-octynyl)pyridine hydrochloride was prepared in the following manner.

8a. First, 3-(3-(S)-pyrrolidinyloxy)-5-(1-octyny)pyridine was synthesized. 3-(1-N-BOC-3-(S)-pyrrolidinyloxyl)-5-bromopyridine from Example 2a (0.3 g, 0.87 mmol) was dissolved in CH₂Cl₂ (10 mL). 1-Octyne (0.3 mL, 1.75 mmol), palladium(II) bis(triphenylphosphine) chloride (0.02 g), CuI (catalytic amount) and triethylamine (0.5 mL) were added. The mixture was stirred and refluxed for 48 hours. After cooling to room temperature, solvent was evaporated and the residue was chromatographed (silica gel; hexane: EtOAc, 10:1 to 5:1) to afford an oil (0.32 g, 98%). MS (DCl/NH₃): m/z 374 (M+H)⁺. ¹H NMR(CDCl₃, 300 MHz) δ0.91 (t, J=7.0 Hz, 3H), 1.13 (m, 2H), 1.45-1.50 (m, 2H), 1.47 (s, 9H), 1.60 (q, J=7.0 Hz, 2H), 1.62-1.70 (m, 2H), 2.35 (brs, 2H), 2.43 (t, J=7.0 Hz, 2H), 3.44-3.64 (m, 4H), 4.92 (brs, 1H), 7.18 (s, 1H), 8.20 (brs, 2H). 8b. TFA (1 mL) was added to a solution of3-(1-N-BOC-3-(S)-pyrrolidinyloxy)-5-(1-octynyl)pyridine (0.32 g, 0.86 mmol) in CH₂Cl₂ (2 mL) at 0C. The mixture was stirred at room temperature for 1 hour. Solvent was evaporated and the residue was basified with potassium carbonate. The aqueous layer was extracted with CH₂Cl₂ three times. The organic layer was dried (MgSO₄), concentrated and chromatographed (silica gel; CH₂Cl₂: MeOH, 10:1) to afford the oil, 3-(3-pyrrolidinyloxy)-5-(1-octynyl)pyridine hydrochloride (0.2 g, 73%). MS (DCl/NH₃): m/z 273 (M+H)⁺. ¹H NMR (CDCl₃, 300 MHz) δ0.92 (t, J=7.0 Hz, 3H), 1.30-1.36 (m, 3H), 1.40-1.48 (m, 2H), 1.58-1.64 (m, 2H), 2.00-2.20 (m, 4H), 2.42 (t, J=7.0 Hz, 2H), 3.04 (m, 1H), 3.16-3.30 (m, 2H), 4.88 (m, 1H), 7.16 (m, 1H), 8.18 (m, 1 1H), 8.04 (m, 1H). The free base was converted to salt with 1.0 HCl in ether. A light yellow hygroscopic solid was obtained: mp 66-68° C. MS: (DCl/NH₃): m/z 273 (M+H)⁺. ¹H NMR (D₂O, 300 MHz) δ0.90 (s, 3H), 1.32 (s, 4H), 1.40-1.38 (m, 2H), 1.64 (m, 2H), 2.40 (m, 2H), 2.42-2.56 (m, 2H), 3.50-3.60 (m, 2H), 3.60-3.78 (m, 2H), 5.40 (m, 1H), 8.02 (s, 1H), 8.39 (s, 1H), 8.41 (s, 1H). Calc'd. Anal. for C₁₇H₂₄N₂O.2.7 HCl.0.1 H₂O: C, 54.80; H, 7.28; N, 7.52. Found: C, 54.90; H, 7.53; N, 7.47.[α]_{D}^{≦}−9.4 (c 0.4, MeOH).

### EXAMPLE 9

3-(3-(R)-pyrrolidinyloxy)-5-(l -octynyl)pyridine hydrochloride was synthesized by the same procedure as described in Example 8, except the 3-(1-N-BOC-3-(S)-pyrrolidinyloxy)-5-bromopyridine was replaced with 3-(1-N-BOC-3-(R)-pyrrolidinyloxy)-5-bromopyridine. The product was obtained as a yellow hygroscopic syrup. MS (DCl/NH₃): m/z 273 (M+H).+¹H NMR (D₂O, 300 MHz) δ0.90 (s, 3H), 1.22-1.40 (m, 4H), 1.40-1.50 (m, 2H), 1.55-1.70 (m, 2H), 2.30-2.40 (m, 2H), 2,45-2.55 (m, 2H), 3.48-3.75 (m, 4H), 5.35 (m, 1H), 7.80 (s, 1H), 8.30 (s, 2H). Calc'd. Anal. for C₁₇H₂₄N₂O.2 HCl.0.4 H₂O: C, 57.92; H, 7.66; N, 7.95. Found: C, 57.81; H, 7.36; N, 7.76. [α]_{D}²³ −17.9 (c 1.2, MeOH).

### EXAMPLE 10

3-(1-N-Methyl-3-(R)-pyrrolidinyloxy)-5-(1-octynyl)pyridine hydrochloride was obtained as follows.

3-(3-(R)-pyrrolidinyloxy)-5-(1-octynyl)pyridine (prepared in Example 9, 0.19 g, 0.51 mmol) was treated with formic acid (88%, 2.6 mL) and formaldehyde (37%, 5.2 mL) at 70° C. for 6 hours. After cooling to room temperature, the reaction mixture was basified and extracted with CH₂Cl₂ three times. The organic layer was dried (MgSO₄), filtered, concentrated and chromatographed (silica gel; CH₂C1₂/MeOH, 10:0.2 to 10:1) to afford the free base as an oil (85 mg, 58%). The free base was converted to HCl salt with 1.0 M HCl in ether as a light yellow hygroscopic solid: mp 133-135° C. MS (DCl/NH₃): m/z 287 (M+H)+. ¹H NMR (D₂O, 400 MHz) δ0.9 (t, J=7.0 Hz, 3H), 1.25-1.40 (m, 4H), 1.40-1.52 (m, 2H),1.55-1.70 (m, 4H), 2.10 (m, 1H), 2.38-2.50 (m, 2H), 2.55 (s, 3H), 2.88-3.00 (m, 2H), 4.70-5.00 (m, 2H), 7.8 (m, 1H), 8.30 (m, 1H), 8.40 (s, 1H). Calc'd. Anal. for C₁₈H₂₆N₂O.1.7 HCl: C, 62.05; H, 8.01; N, 8.04. Found: C, 62.01; H, 8.06; N, 7.91. [α]_{D}²³ −18.4 (c 1.9, MeOH).

### EXAMPLE 11

3-(3-(S)-pyrrolidinyloxy)-5-(5-pyrimidyl)pyridine hydrochloride was obtained in the following manner.

3-(1-BOC-3-(S)-pyrrolidinyloxy)-5-bromopyridine from Example 2a (0.27 g, 0.78 mmol) was mixed with tributyl(pyrimidyl)tin (0.43 g, 1.2 mmol) and tetrakis(triphenylphosphine)palladium (0) (20 mg) in toluene (10 mL), then was stirred at reflux for 16 hours. After the reaction mixture was cooled to room temperature, solvent was evaporated and the residue was chromatographed (silica gel; hexane:EtOAc, 2:1 to 0: 1) to give free base as an oil (0.19 g, 72%). MS (DCl/NH₃): m/z 343 (M+H)⁺. ¹H NMR (CDCl₃, 300 MHz) δ1.47 (s, 9H), 1.58 (m, 1H), 2.18-2.28 (m, 2H), 3.56 (m, 1H), 2.64-2.72 (m, 2H), 5.02 (m, 1H), 7.38 (s, 1H), 8.38 (d, J=2.7 Hz, 1H), 8.47 (s, 1H), 8.98 (d, J=12.2 Hz, 2H), 9.30 (d, J=19.0 Hz, 1H). The compound was converted to salt with 4.0 M HCl in 1,4-dioxane. The salt was obtained as a light yellow hygroscopic solid: mp 60-62° C. MS (DCl/NH₃): m/z 243 (M+H)⁺. ¹H NMR (D₂O, 300 MHz) d 2.42-2.50 (m, 2H), 3.56-3.64 (m, 2H), 3.66-3.83 (m, 2H), 5.58 (m, 1H), 8.48 (m, 1H), 8.68 (m, H), 8.85 (m, 1H), 9.09 (m, 2H), 9.30 (m, 1H). Calc'd. Anal. for C₁₃H,₄N₄O.3.2 HCl: C, 43.50; H, 4.83; N, 15.61. Found: C, 43.69; H, 4.86; N, 15.25. [α]_{D}²³=−13.5 (c 0.6, MeOH).

### EXAMPLE 12

3-(1-Methyl-3-(S)-pyrrolidinyloxy)-5-(3-pyridyl)pyridine hydrochloride was prepared as follows. 12a. First, 3-(1-BOC-3-(S)-pyrrolidinyloxy)-5-(3-pyridyl)pyridine was synthesized. 3-(1-BOC-3-(S)-pyrrolidinyloxy)-5-bromopyridine (0.25 g, 0.73 mmol) was treated with tributyl(3-pyridyl)tin (0.40 g, 1.1 mmol) in toluene (10 mL) according to the procedure described in Example 11. The desired product was obtained as an oil(1 17 mg, 47%). MS (DCl/NH₃): m/z 342 (M+H)⁺.

12b. 3-(1-methyl-3-(S)-pyrrolidinyloxy)-5-(3-pyridyl)pyridine hydrochloride was then obtained by treating the compound of step 12a (0.12 g, 0.34 mmol) with formic acid (88%, 1.6 mL) and formaldehyde (70%, 3.2 mL) at 70° C. for 6 hours. The mixture was basified and extracted with CH₂Cl₂ three times. The organic layer was dried (MgSO₄), filtered, concentrated and chromatographed (silica gel; CH₂Cl₂: MeOH, 10:0.2 to 10: 1) to afford the free base an oil (0.07 g, 81%). MS (DCl/NH₃): m/z 256 (M+H)⁺. The free base was converted to HCl salt with 1.0 M HCl in ether. The salt was obtained as a sticky hygroscopic solid. MS (DCl/NH₃): m/z 256 (M+H)⁺. ¹H NMR (D₂O, 300 MHz) δ2.10-2.40 (brs, 2H), 2.30 (m, 1H), 2.86 (s, 3H), 3.22 (brs, 1H), 3.60-3.80 (brs, 2H), 5.22 (m, 1H), 7.48 (m, 1H), 7.56 (m, 1H), 8.06 (m, 1H) 8.16 (m, 1H), 8.30 (m, 1H), 8.44 (m, 1H), 8.68 (m, 1H). Calc'd. Anal. for C₁₅H₁₇N₃O.2HCl.4H₂O: C, 50 97; H, 6.22; N, 11.89. Found: C, 51.12; H, 6.31; N, 11.81. [α]_{D}²³=−7.4 (c 1.4, MeOH).

### EXAMPLE 13

3-(3-(R)-Pyrrolidinyloxy)-5-(5-pyrimidinyl)pyridine hydrochloride was synthesized by utilizing the same procedure as described in Example 11, except the 3-(1-BOC-3-(S)-pyrrolidinyloxy)-5-bromopyridine was replaced with 3-(1-BOC-3-(R)-pyrrolidinyloxy)-5-bromopyridine. The title product was obtained as light yellow hygroscopic solid: mp 67-69° C. MS (DCl/NH₃): m/z 243 (M+H)⁺. ¹H NMR (CD₂O, 300 MHz) 6 2.45-2.52 (m, 2H), 3.56-3.64 (m, 2H), 3.72 (m, 1H), 5.60 (m, lH), 8.52 (m, 1H), 8.69 (m, ^{H),} 9.19 (s, 2H), 9.29 (d, J=15.0 Hz, 1H). Calc'd. Anal. for C₁₃H₁₄N₄O.3.3 HCl: C, 43.06; H, 4.81; N, 15.45. Found: C, 43.03; H, 5.15; N, 15.65. [α]_{D}²³=−14.3 (c 0.7, MeOH).

### EXAMPLE 14

3-(3-(S)-Pyrrolidinyloxy)-5-(3-aminophenyl)pyridine hydrochloride was prepared as follows. 3-(1-BOC-3-(S)-pyrrolidinyloxy)-5-bromopyridine (0.25 g, 0.73 mmol) in toluene (10 mL) was mixed with 3-aminophenyl boronic acid (0.23 mg, 1.46 mmol), tetrakis(triphenylphosphine) palladium (0) (20 mg) and 2N Na₂CO₃solution (1 mL). The mixture was stirred and refluxed for 16 hours. After cooling to room temperature, solvent was evaporated. The residue was chromatographed (silica gel; CH₂Cl₂: MeOH, 10:0.5 to 10:1) to afford the product as an oil (0.18 g, 70%). MS (DCl/NH₃): m/z 356 (M+H)⁺. The compound was converted to the salt with 4.0 M HCl in 1,4-dioxane as a white solid: mp 208° C.(dec). MS (DCl/NH₃): mlz 256 (M+H)⁺. ¹H NMR (D₂O, 300 MHz) 82.34-2.44 (m, 2H), 3.52-3.76 (m, 4H), 5.42 (m, 1H), 7.06 (m, 1H), 7.22-7.34 (m, 2H), 7.44 (m, 1H), 7.82 (s, 1H), 8.37 (s, 1H), 8.54 (s, 1H). Calc'd. Anal. for Cl₅H₁₇N₃O₃.HCl.1.2 H₂O: C, 46.64; H, 5.84; N, 10.88. Found: C, 46.76; H, 5.80; N, 10 65. [α]_{D}²³ −8.0 (c 0.5, MeOH).

### EXAMPLE 15

3-(3-(S)-Pyrrolidinyloxy)-5-phenylpyridine hydrochloride was prepared in the following manner.

15a. 3-(1-Benzyl-3-(S)-pyrrolidinyloxy)-5-phenylpyridine was first prepared as follows. 3-(1-Benzyl-3-(S)-pyrrolidinyloxy)-5-bromopyridine (0.50 g, 1.50 mmol) in toluene (10 mL) was mixed with phenyl boronic acid (0.27 mg, 2.24 mmol), tetrakis(triphenylphosphine) palladium (0) (50 mg) and 2N Na₂CO₃ (3.5 mL). The reaction mixture was stirred and refluxed for 16 hours. After cooling to room temperature, solvent was evaporated. The residue was chromatographed (silica gel; hexane/EtOAc, 3:1 to 0:1) to afford an oil (0.31 g, 62%). MS (DCl/NH₃): m/z 332 (M+H)⁺.

15b. 3-(3-(S)-Pyrrolidinyloxy)-5-phenylpyridine was obtained by hydrogenating the compound obtained in step 15a (0.30 g, 0.90 mmol) in EtOAc in the presence of Pd-C (0.10 g, 10%) for 3 days. After filtration, solvent was evaporated. 40 mg of product was obtained (18%). MS (DCl/NH₃): m/z 240 (M+H)⁺. The free base was converted to the salt with 1.0 M HCl in ether as a light yellow solid: mp 60° C. (dec). MS (DCl/NH₃): m/z 241. ¹H NMR (D₂O, 300 MHz) δ2.35-2.48 (m, 2H), 3.50-3.60 (m, 3H), 3.72 (m, 1H), 5.40 (m, 1H), 7.42-7.60 (m, 3H), 7.64-7.76 (m, 2H), 8.32 (s, 1H), 8.56 (s, 1H). Calc'd. anal. for C₁₅H₁₆N₂O.1.7 HCl.H₂O: C, 56.25; H, 6.20; N, 8.67. Found: C, 56.49; H, 6.45; N, 8.27. [α]_{D}²³ 28.8 (c 0.2, MeOH).

### EXAMPLE 16

3-(1-Methyl-3-(S)-pyrrolidinyloxy)-5-phenylpyridine hydrochloride was obtained as follows.

16a. 3-(1-BOC-3-(S)-pyrrolidinyloxy)-5-phenylpyridine 3-(1-BOC-3-(S)-pyrrolidinyloxy)-5-bromopyridine from Example 2a (0.2 g, 0.58 mmol) was reacted with phenyl boronic acid (0.14 mg, 1.17 mmol), tetrakis(triphenylphosphine) palladium (0) (20 mg), 2N Na₂CO₃(1.2 mL) in toluene (10 mL) according the procedure as described in Example 14. The desired product (199 mg, 100%) was obtained as an oil. MS (DCl/NH₃): m/Z 341 (M+H)⁺.

16b. 3-(1-Methyl-3-(S)-pyrrolidinyloxy)-5-phenylpyridine hydrochloride was then synthesized as follows. 3-(1-N-BOC-3-(S)-pyrrolidinyloxyl)-5-phenylpyridine (0.20g, 0.6 mmol) was mixed with formic acid (3.3 mL) and formaldehyde (70%, 6.6 mL), then stirred at 70° C. for 6 hours. The reaction mixture was basified and the aqueous layer was extracted with CH₂Cl₂ three times. The organic layer was dried (MgSO₄), filtered, concentrated and chromatographed (silica gel; CH₂Cl₂: MeOH, 10:0.5 to 10:1) to give free base an oil (0.12 g, 80%). MS (DCl/NH₃): m/z 255 (M+H)⁺. The free base was converted to salt with 1.0 M HCl in ether. mp 88-90° C. MS (DCl/NH₃): n/z 255 (M+H)⁺. ¹H NMR (CDCl₃, 300 MHz) 52.40 (m, 1H), 2.64 (m, 1H), 3.06 (s, 3H), 3.20-3.60 (br, m, 2H), 3.70-3.98 (br, m, 2H), 5.40 (m, 1H), 7.50-7.62 (m, 3H), 7.66-7.77 (m, 3H), 8.25 (d, J=2.7 Hz, 1H), 8.50 (d J=1.7 Hz, 1H). Calc'd. Anal. For C₁₆H₁₈N₂O 1.7 HCl.O.3 H₂O: C, 59.73; H, 6.36; N, 8.71. Found: C, 59.84; H, 6.70; N, 8.50. [α]_{D}²³ −8.7 (c 0.5, MeOH).

### EXAMPLE 17

3-(3-(R)-Pyrrolidinyloxy)-5-phenylpyridine hydrochloride was synthesized by the same procedure as described in Example 16, except the 3-(1-N-BOC-3-(S)-pyrrolidinyloxy)-5-bromopyridine was replaced with 3-(1-N-BOC-3-(R)-pyrrolidinyloxy)-5-bromopyridine. The title product was obtained as light yellow hygroscopic solid: mp 60° C.(dec). MS (DCl/NH₃): m/z 241 (M+H)⁺. ¹H NMR (D₂O, 300 MHz) δ2.42-2.50 (m, 2H), 3.54-3.64 (m, 3H), 3.80 (m, 1H), 5.54 (m, 1H), 7.60-7.68 (m, 3H), 7.72-7.80 (m, 2H), 8.30 (m, 1H), 8.50 (m, 1H), 8.70 (m, 1H). Calc'd Anal. for C₁₅H₁₆N₂O.3.2 HCl: C, 50.47; H, 5.42; N, 7.85. Found: C, 50.38; H, 5.38; N, 7.46. [α]_{D}²³ −23.7 (c 0.3, MeOH).

### EXAMPLE 18

3-(3-(S)-Pyrrolidinyloxy)-5-thienylpyridine hydrochloride was prepared in the following manner.

18a. 3-(1-N-Benzyl-3-(S)-pyrrolidinyloxy)-5-thienylpyridine was prepared as follows. 3-(1-N-Benzyl-3-pyrrolidinyloxy)-5-bromopyridine from Example 1a, (0.75 g, 2.24 mmol) in toluene (15 mL) was mixed with 2-thienyl boronic acid (0.72 mg, 5.61 mmol), tetrakis(triphenylphosphine) palladium (0) (80 mg) and 2N Na₂CO₃(5.2 niL). The reaction mixture was stirred at reflux for 16 hours. After cooling to room temperature, solvent was evaporated. The residue was chromatographed (silica gel; hexane/EtOAc, 3:1 to 0:1) to give free base as an oil (0.30 g, 39%). MS (DCl/NH₃): m/z 339 (M+H)⁺.

18b. 3-(3-(S)-Pyrrolidinyloxy)-5-thienylpyridine hydrochloride was then formed by adding to an ice cold solution of the compound formed in step 18a (0.25 mg, 0.74 mmol) in CH₂Cl₂. 1-chloroethyl chloroformate (0.4 mL, 3.7 mmol) slowly. The reaction mixture was then warmed to room temperature and stirred for 1 hour. Next, saturated aqueous NaHCO₃was added. The organic layer was washed with brine, dried (MgSO₄) and concentrated. MeOH (5 mL) was added to the resultant residue, which was then was refluxed for 1 hour. The mixture was basified with 10% NaOH in CH₂Cl₂. The organic layer was dried (MgSO₄), filtered, concentrated and chromatographed (silica gel; CH₂Cl₂/MeOH, 10:0.5 to 10:1) to afford the free base as an oil (25 mg, 21%). MS (DCl/NH₃): m/z 247 (M+H)⁺. The free base was converted to HCl salt with 1.0 M HCl in Et₂O. MS (DCl/NH₃): m/z 247 (M+H)⁺. ¹H NMR (300 MHz, D₂O) δ2.36-2.42 (m, 2H), 3.56-3.60 (m, 3H), 3.70 (m, 1H), 5.40 (m, 1H), 7.23 (dd, J=3.5, 5.5 Hz, 1H), 7.56 (d, J=3.5, 1H), 7.60 (m, 1H), 8.20 (brs, 1H), 8.54 (brs, 1H). Calc'd. Anal. for Cl₃H₁₄N₂OS 2.6.HCl.1.88 H₂O: C, 41.64; H, 5.47; N, 7.40. Found: C, 42.03; H, 5.24; N, 7.00. [α]_{D}²³ 31.4 (c 0.1, MeOH).

### EXAMPLE 19

3-(3-(R)-Pyrrolidinyloxy)-5-thienylpyridine hydrochloride was obtained by the following method. 3-(1-N-BOC-3-(R)-Pyrrolidinyloxy)-5-bromopyridine from Example 13 (0.70 g, 2.05 mmol) in toluene (15 mL) was mixed with 2-thienyl boronic acid (1.05 g, 8.25 mmol), tetrakis(triphenylphosphine) palladium (0) (60 mg) and 2N Na₂CO₃(2.3 mL). The reaction mixture was stirred at reflux for 16 hours. After the mixture was cooled to room temperature, solvent was evaporated. The residue was chromatographed (silica gel; hexane/EtOAc, 3:1 to 1:1) to furnish the free base as an oil (0.60 g, 86%). MS (DCl/NH₃): m/z 347 (M+H)⁺. The free base was converted to HCl salt with 4.0 M HCl in 1,4-dioxane to afford the title compound as a light green solid: mp 201-203° C. MS (DCl/NH₃): m/z 247 (M+H)⁺. ¹H NMR (D₂O, 300 MHz) δ2.36-2.46 (m, 2H), 3.50-3.68 (m, 3H), 3.72 (m, 1H), 5.44 (m, 1H), 7.23 (dd, J=3.5, 4.5 Hz, 1H), 7.61 (m, 2H), 7.90 (m 1H), 8.27 (d, J=3.0 Hz, 1H), 8.60 (d, J=2.0 Hz, 1H). Calc'd. Anal. for C₁₃H₁₄N₂O S.2.2 HCl: C, 47.82; H, 5.00; N, 8.58. Found: C, 47.73; H, 5.10; N, 8.36. [α]_{D}²³ −24.4 (c 0.9, MeOH).

### EXAMPLE 20

3-(1-N-Methyl-3-(R)-pyrrolidinyloxy)-5-thienylpyridine hydrochloride was obtained in the following manner. 3-(1-N-BOC-3-(R)-pyrrolidinyloxy)-5-thienylpyridine from Example 19 (0.37 g, 1.07 mmol) was treated with formaldehyde (10 mL, 37%) in formic acid (5 mL) at 70° C. for 16 hours. After cooling to room-temperature, the reaction mixture was basified and extracted with CH₂Cl₂ four times. The organic layer was dried (MgSO₄), concentrated and chromatographed (silica gel; CH₂Cl₂/MeOH, 10:0.1 to 10:0.5 to 10: 1) to afford free base as an oil (0.16 g, 56%). MS (DC1/NH₃): m/z 261 (M+H)⁺. The free base was then converted to an HCl salt with 1.0 M HCl in ether. MS (DCl/NH₃): m/z 261 (M+H)⁺. ¹H NMR (D₂O, 300 MHz) δ2.25-2.80 (br,m, 2H), 3.05 (s, 3H), 3.22-3.52 (br, m, 2H), 3.82-4.04 (m, 2H), 5.40 (m, 1H), 7.22 (dd, J=3.5, 5.0 Hz, 1H), 7.57 (m, 2H), 7.70 (s, 1H), 8.18 (d, J=2.0 Hz, 1H), 8.53 (d, J=2.0 Hz, 1H). Calc'd. Anal. for C₁₄H₁₆N₂OS.1.8 HCl: C, 51.58; H, 5.50; N, 8.59. Found: C; 51.45; H, 5.56; N, 8.38. [α]_{D}²³ −24.8 (c 0.4, MeOM).

### EXAMPLE 21

3-(3-(S)-Pyrrolidinyloxy)-5-(4-chlorophenyl)pyridine hydrochloride was obtained in the following manner. 3-(1-N-BOC-3-( S)-pyrrolidinyloxy)-5-bromopyridine from Example 2a (0.22 g, 0.64 mmol) in toluene (5 mL) was mixed with 4-chlorophenyl boronic acid (0.20 g, 1.28 mmol), tetrakis(triphenylphosphine) palladium (0) (22 mg) and 2N aqueous Na₂CO₃(1.3 mL). According to the procedure described in Example 14, the product was obtained (0.24 g, 98% ) as a light yellow solid: mp 65° C. (dec). MS (DCl/NH₃): m/z 275 (M+H)⁺. ¹H NMR (D₂O, 300 MHz) δ2.38-2.50 (m, 2H), 3.54-3.80 (m, 4H), 5.42 (m, 1H), 7.56 (d, J=8.4 Hz, 2H), 7.65 (s, 1H), 7.70 (d, J=8.2 Hz, 2H), 8.29 (s, 1H), 8.48 (s, 1H). Calc'd. Anal. for C₁₅H₁₅ClN₂O.2.65 HCl.1.04 H₂O: C, 46.18; H, 5.10; N, 7.18. Found: C, 46.57; H, 5.32; N, 6.78. [α]_{D}²³ −7.2 (c 0.9, MeOH).

### EXAMPLE 22

3-(3-(S)-Pyrrolidinyloxy)-5-bromo-6-chloropyridine hydrochloride was obtained in the following manner.

22a. 3-(1-N-Benzyl-3-(R)-pyrrolidinyl)-tosylate was first made as follows. To a solution of 1-benzylpyrrolidinyl-3-ol (5.0 g., 28.2 mmol) in CH₂Cl₂ was added triethylamine (8.7 mL, 62.0 mmol) and tosyl chloride (10.8 g, 56.5 mmol) at room temperature. After 16 hours, the reaction was quenched with saturated aqueous NH₄Cl. The organic layer was dried (MgSO₄), concentrated and chromatographed (silica gel, hexanes/EtOAc) 5:1 to 2.5:1) to afford an oil (1.52 g., 16%). MS (DCl/NH₃) m/z 332 (M+H)⁺.

22b. 3-(1-N-Benzyl-3-(S)-pyrrolidinyloxy)-6-chloro-5-bromopyridine was then synthesized as follows. 3-(1-N-Benzyl-3-(R)-pyrrolidinyl)-tosylate (1.52 g., 4.50 mmol) was mixed with 2-chloro-3-bromo-5-hydroxypyridine (1.45 g., 6.96 mmol) and potassium hydroxide (487 mg., 8.70 mmol) in DMF. The mixture was stirred at 70° C. for 20 hours. Solvent was washed with H₂O/brine (1:1, four times) in EtOAc. The organic layer was dried (MgSO₄), concentrated and chromatographed (hexanes/EtOAc), 5:1 to 2:1) to afford an oil (1.55 g., 94%). MS (DCl/NH₃) m/z 369 (M+H)⁺.

22c 3-(1-N-BOC-3-(S)-pyrrolidinyloxy)-5-bromo-6-chloropyridine was then synthesized according to the same procedure as 2a, except that 3-(1-N-Benzyl-3-(S)-pyrrolidinyloxy)-6-chloro-5-bromopyridine replaced 3-(1-N-Benzyl-3-(S)-pyrrolidinyloxy)-5-bromopyridine. MS (DCl/NH₃)m/z 379 (M+H)⁺.

22d 3-(3-(S)-pyrrolidinyloxy)-5-bromo-6-chloropyridine hydrochloride was made as follows. The product from 22c was converted to the HCl salt with 4.0 M HCl in 1,4-dioxane to afford the title compound as a light yellow solid: m.p. 178-180° C. ¹H NMR (D₂O, 300 MHz) δ2.30-2.50 (m, 2H), 3.50-3.75 (m, 4H), 5.30 (s, 1H), 7.88 (s, 1H), 8.09 (s, lH). MS (DCl/NH₃) m/z 279 (M+H)⁺. Calc'd Anal. for C₉H₁₀BrClN₂O).HCl: C, 34.43; H, 3.53; N, 8.92. Found: C, 34.21; H, 3.55; N, 8.75. [α]_{D}²³ 27.5 (c 0.3, MeOH).

### EXAMPLE 23

3-(1-N-Methyl-3-(S)-pyrrolidinyloxy)-5-bromo-6-chloropyridine hydrochloride was prepared as follows. 3-(1-N-BOC-3-(S)-pyrrolidinyloxyl)-5-bromo-6-chloropyridine from Example 22 (0.24 g, 0.82 mmol) was heated with formic acid (4.1 mL) and formaldehyde (8.2 mL) at 70° C. for 16 hours. The reaction mixture was basified and then extracted with CH₂Cl₂ three times. The organic layers were combined, dried (MgSO₄), filtered, concentrated and chromatographed (silica gel; CH₂Cl₂/MeOH, 10:0.5 to 10: 1) to afford the free base as an oil (0.13 g, 56%). MS (DCl/NH₃): m/z 293 (M+H)⁺. The free base was converted to the HCl salt with 1.0 M HCl in ether. A white solid was obtained: mp 180° C.(dec). ¹H NMR (D₂O, 300 MHz) δ2.25-2.80 (m, 2H), 3.04 (s, 3H), 3.50 (brs, 2H), 3.75-4.00 (m, 2H), 5.22 (m, 1H), 7.87 (s, 1H), 8.08 1H). MS (DCl/NH₃): m/z 293 (M+H)⁺. Calc'd. Anal. for C₁₀H₁₂BrClN₂O.1.2 HCl: C, 35.82; H, 3.97; N, 8.35. Found: C, 35.79; H, 3.96; N, 8.30. [α]_{D}²³ 42.3 (c 0.1, MeOH).

### EXAMPLE 24

3-(1-N-Methyl-3-(S)-pyrrolidinyloxy)-5-(2-(4-pyridyl)ethenyl)-6-chloropyridine hydrochloride was formed in the following manner.

3-(1-N-BOC-3-(S)-pyrrolidinyloxyl)-5-bromo-6-chloropyridine from Example 22 (0.30 g, 0.79 mmol) was dissolved in acetonitrile (10 mL). 4-Vinylpyridine (0.17 niL, 1.6 mmol), palladium (II) acetate (30 mg), tri-o-tolylphosphine (0.15 g) and triethylamine (0.27 mL) were added. According to the procedure described in Example 2a, the desired product (0.21 g, 66%) was obtained as an oil (66%). This compound was converted to the salt with in 4.0 M HCl mp 106-108° C. MS (DC1/NH₃): m/z 315 (M+H)⁺. ¹H NMR (D₂O, 300 MHz) δ2.60 (m, 1H), 2.42 (m, 1H), 2.51 (s, 3H), 2.55-2.75 (brs, 1H), 2.90-3.04 (m, 3H), 4.95 (m, 1H), 7.00 (d, J=16.5 Hz, 1H), 7.39-7.42 (m, 2H), 7.48 (m, 1H), 7.60 (d, J-16.0 Hz, 1H), 8.10 (d, J=3.0 Hz, 1H), 8.70-8.75 (m, 2H). Calc'd. Anal. For C₁₇H₁₈ClN₃O.2.5HCl.19H₂O. C, 46.28; H, 5.55; N, 9.52. Found: C, 46.34; H, 5.87; N, 9.51. [α]_{D}²³ 13.6 (c 0.55, MeOH).

### EXAMPLE 25

3-Bromo-2-chloro-5-(3-pyrrolidinylmethoxy)pyridine toluenesulfonate was obtained in the following manner.

25a. 3-Bromo-2-chloro-5-(1-benzyl-3-pyrrolidinylmethoxy)pyridine was obtained in the following manner. To a solution of diethyl azodicarboxylate (1.84 mL, 11.7 mmol) in THF (20 mL) was added triphenylphosphine (3 g, 11.7 mmol) at 0° C., and the reaction mixture was stirred for half an hour. l-Benzyl-3-pyrrolidinylmethanol (1 g, 5.2 mmol, available as described in J. Chem. Soc. 1959, 851) and 5-bromo-6-chloropyridine-3-ol (2.43 g, 11.7 mmol; prepared according to V. Koch and S. Schnatterer, Synthesis, 1990, 499-501)) were then added. The reaction mixture was slowly warmed up to room temperature overnight. Solvent was removed, and the residue was chromatographed on a silica gel column, eluting with ethyl acetate:methylene chloride 1:1 to afford the title compound (1.05g, 53%). MS (DCl/NH₃): m/Z 381(M+H)⁺, 383 (M+3H)⁺. ¹H NMR (D₂O, 300 MHz) δ2.15 (brs, 1H), 2.5-2.9 (m, 6H), 3.65-3.86 (m, 2H), 3.79-4.01 (m, 2H), 7.30-7.73 (m, 6H), 8.01 (d, J=3.0, 1H).

25b. 3-Bromo-2-chloro-5-(3-pyrrolidinylmethoxy)pyridine toluenesulfonate was obtained in the following manner. To 3-bromo-2-chloro-5-(1-benzyl-3-pyrrolidinylmethoxy)pyridine from step 25a (480 mg, 1.26 mmol) was added 1-chloroethyl chloroformate (0.54 mL, 5.05 mmol) in chloroform at room temperature, and the mixture was stirred at reflux for 5 hours. Methanol was then added to the reaction mixture, and the resultant solution was allowed to reflux for an additional 2 hours. The volatiles were then removed under vacuum. The residue was neutralized with K₂CO₃ to pH 8, then extracted with methylene chloride, which was dried over MgSO4 and concentrated. The residue was chromatographed on a silica gel column, eluting with methylene chloride:methanol:NH40H 10:1:0.1 to afford the free base of the title compound. The base was converted to the salt by treatment with p-toluenesulfonic acid in ethanol to give the title compound (142 mg). mp 105-107° C. MS (DCl/NH₃): m/z 291 (M+H)+, 308 (M+NH₄)+. ¹H NMR (D₂O, 300 MHz) δ1.96 (m, 1H), 2.28 (m, 1H), 2.38 (s, 3H), 2.95 (m, 1H), 3.20-3.60 (m, 4H), 4.09 (dd, J=6.2, 9.2 Hz, 1H), 4.15 (dd, J=5.6, 10 Hz, 1H), 7.35 (d, J=8.1 Hz, 2H), 7.68 (d, J=8.1 Hz, 2H), 7.79 (d, J=2.6 Hz, 1H), 8.02 (d, J=2.9 Hz, 1H). Anal. Calc'd for C₁₀H₁₂N₂OBrCl.TsOH: C, 44.03; H, 4.35; N, 6.04. Found: C, 44.42; H, 4.52; N, 5.68.

### EXAMPLE 26

3-Bromo-2-chloro-5-(1-methyl-3-pyrrolidinylmethoxy) pyridine toluenesulfonate was synthesized as follows. The compound obtained by the procedure of Example 25 (150 mg, 0.52 mmol) was stirred with excess paraformaldehyde (4 mL) in formic acid (2 mL). The reaction was stirred at 60° C. for 16 hours. The reaction mixture was then basified with solid potassium carbonate and the resultant solution was diluted with water. The aqueous phase was extracted with methylene chloride (5×5 mL). These extracts were dried (MgSO4), filtered, and concentrated in vacuo. The resultant oil was then purified by flash silica gel chromatography (CHCl₃:MeOH:NH₄OH/10:1:0.1), yielding the pure product (49.mg, 47% yield), which was dissolved in ethanol and converted to the tosylate salt in a similar manner as that of Example 25b. MS (DC1I/NH₃): m/e 305 (M+H)⁺, 307 (M+2H)⁺. ¹H NMR (D₂O, 300 MHz) δ2.12 (m, 1H), 2.27 (m, 1H), 2.37 (s, 3H), 2.95 and 2.97 (s, 3H), 3.13-3.40 (m, 3H), 3.61-3.90 (m, 2H), 4.0-4.20 (m, 2H), 7.34 (d, J=8.1 Hz, 2H), 7.66 (d, J=8.1 Hz, 2H), 7.76 (d, J=3 Hz, 1H), 8.01 (d, J=3 Hz, 1H). Anal. Calc'd for C₁₁H₁₄N₂OBrCl.1.1 TsOH: C, 45.19; H, 5.03; N, 5.64. Found: C, 44.92; H, 5.30; N, 5.30.

### EXAMPLE 27

5-Methyl-3-(3-(pyrrolidinyl)methoxy)pyridine was prepared in the following manner.

27a. First, 5-bromo-3-((3-pyrrolidinyl)methoxy)pyridine was synthesized. 1-Benzyl-3-pyrrolidinemethanol (0.98 g, 5.2 mmol) was carefully added to the suspension of sodium hydride (0.41 g, 60% in mineral oil, 10.3 mmol) in anhydrous DMF (5 mL). After stirring at room temperature for 0.5 hour, 3,5-dibromopyridine (1.7 g, 7.5 mmol) was added, and the reacting mixture was stirred at room temperature for 3 days. Another 5.0 mL of water was added, and the solvents were removed under reduced pressure. Again, water (5.0 mL) was added, and the slurry was washed extensively with EtOAc (4×40 mL). The combined organic layers were dried (Mg₂SO₄), filtered and concentrated. The crude product was purified by flash chromatography on silica gel eluting with ethyl acetate to provide 0.92 g (52% yield) of the title compound. MS (DCl/NH₃): m/e 347 with ⁷⁹Br and 349 (M+H)⁺ with ⁸¹Br. ¹H NMR (CDCl₃, 300 MHz) δ2.1-2.18 (m, 1H), 2.41-2.56 (m, 2H), 2.60-2.80 (m, 4H), 3.61-3.74 (m, 2H), 3.90-3.96 (m, 2H), 7.28-7.38 (m, 6H), 8.21 (d, J=2.7 Hz, 1H), 8.27 (d, J=3 Hz, 1H).

27b. 5-Methyl-3-(1-benzyl-3-pyrrolidinylmethoxy)pyridine was prepared in the following manner. 5-Bromo-3-(1-benzyl-3-pyrrolidinylmethoxy)pyridine from step 27a (360 mg, 1 mmol) and [1,3-bis(triphenylphosphino)propane]nickle(II) chloride (5 mg) were dissolved in THF (8 mL) at room temperature. Methylmagnesium bromide (3.0 M, 0.81 mL, 2.5 mmol) was added and the mixture allowed to stir for 16 hours. Water was added, and the product was extracted with CHCl₃ (3×50 mL). The combined organic layers were washed with brine, dried (MgSO₄), and concentrated. The residue was chromatographed (silica gel; EtOAc/MeOH/NH₄OH, 10:1:0.1) to afford the desired product (67 mg, 23%). MS (DCl/NH₃): m/z 283 (M+H)⁺: ¹H NMR (CDCl₃, 300 MHz) δ: 2.00-2.20 (m, 2H), 2.33 (s, 3H), 2.40-2.80 (m, 4H), 3.60-3.75 (m, 2H), 3.88-4.00(m, 2H), 6.99 (br s, 1H), 7.28-7.43 (m, 5H), 8.05 (br s, 1H), 8.09(d, J=2.7 Hz, 1H).

27c. 5-Methyl-3-(3-pyrrolidinylmethoxy)pyridine was prepared as follows. 5-Methyl-3-(1-benzyl-3-pyrrolidinylmethoxy)pyridine (162 mg, 0.57 mmol) was dissolved in chloroform (5 mL), and 2-chloroethylchloroformate (0.19 mL, 1.71 mmol) was added. The resultant mixture was stirred at reflux for 5 hours. Solvent was then evaporated and the residue was dissolved in the methanol (5 mL). The mixture was allowed to reflux for an additional 2 hours. After removal of solvent, it was then dissolved in water followed by solid K₂CO₃ to saturate the solution. The mixture was extracted with CHCl₃, and the combined organic layers were washed with brine, dried (MgSO₄), and concentrated. The residue was chromatographed (silica gel; CDCl₃/MeOH/NH₄OH, 10:1:0.1) to afford the desired product (42 mg, 38%). MS (DCl/NH₃): m/z 193 (M+H)⁺, 210 (M+NH₄)⁺. ¹H NMR (CDCl₃, 300 MHz) δ1.90-2.15 (m, 2H, overlap with water peak), 2.32 (s, 3H), 2.60-2.74 (m, 1H), 2.87-2.97 (m, 1H), 3.0-3.30 (m, 3H), 3.88-4.02 (m, 2H), 7.02 (br s, 1H), 8.06 (br s, 1H), 8.12 (d, J=2.5, 1H).

27d. The free base obtained in step 27c (42 mg, 0.22 mmol) was dissolved in ethanol and p-toluenesulfonic acid in ethanol was added dropwise at ambient temperature. The resultant white precipitate was then collected by evaporation of solvent and triturated with three portions of diethyl ether. The hygroscopic solid 5-methyl-3-(3-pyrrolidinylmethoxy)pyridine tosylate was obtained. MS (DCl/NH₃): m/e 193(M+H)⁺. ¹H NMR (CDCl₃, 300 MHz) δ1.90-2.03 (m, 1H), 2.24-2.36 (m, 2H), 2.40 (s, 3H), 2.89-3.01 (m, 1H), 3.29-3.39 (m, 1H), 3.33-3.62 (m, 3H), 4.11-4.25 (m, 2H), 7.37 (d, J=8 Hz, 2H), 7.56 (br s, 1H), 7.68 (d, J=8.4 Hz, 2H), 8.10 (s, 1H), 8.14 (d, J=2.6 Hz, 1H). Anal. Calc'd for C₁₁H₁₆N₂O.1.5 TsOH.1.5 H₂O: C, 54.07; H, 6.54; N, 5.87. Found: C, 53.89; H, 6.39; N, 5.95.

### EXAMPLE 28

5-Phenyl-3-(3-pyrrolidinylmethoxy)pyridine p-toluenesulfonate was prepared according to the following procedure.

28a. First, 5-phenyl-3-(1-methyl-2-(S)-pyrrolidinylmethoxy)pyridine was prepared as follows. To a solution of 5-bromo-3-(1-benzyl-3-pyrrolidinylmethoxy)-pyridine (347 mg, 1 mmol) in toluene (8.0 mL) were added sodium carbonate (2.0 M, 4.0 mL), tetrakis(triphenylphosphine)palladium(0) (45 mg, 0.039 mmol) and phenylboronic acid (146 mg, 1.2 mmol). The reaction mixture was refluxed overnight, and then cooled to room temperature. Water (4 mL) was added, and solid sodium bicarbonate was added until the aqueous layer was saturated. The mixture was extracted with methylene chloride, which was dried over MgSO₄, filtered and concentrated. The residue was chromatographed on a silica gel column, eluting with EtOAc 0:1:9 and to afford a light yellowish oil (75 mg, 21%). ¹H NMR (CDCl₃, 300 MHz) δ1.50-1.75 (m, 2H), 2.12 (m, 1H), 2.40-2.87 (m, 5H), 3.60-3.74 (m, 2H), 4.0 (d, J=6.4 Hz, 2H), 7.23-7.62 (m, 11H), 8.26 (d, J=2.8 Hz, 1H), 8.45 (d, J=1.7 Hz, 1H)

28b. 5-Phenyl-3-(3-pyrrolidinylmethoxy)pyridine p-toluenesulfonate was then synthesized as follows. To 5-phenyl-3-(1-benzyl-3-pyrrolidinylmethoxy)pyridine from step 28a (75 mg, 0.22 mmol) was added 1-chloroethyl chloroformate (0.06 mL, 0.55 mmol) in chloroform (5 mL) at room temperature, and the mixture was stirred at reflux for 3.5 hours. Methanol was then added to the reaction mixture, and the resultant solution was allowed to reflux for an additional 2 hours. The volatiles were then removed under vacuum. The residue was neutralized with K₂CO₃ to pH 8, then extracted with methylene chloride, which was dried over MgSO₄ and concentrated. The residue was chromatographed on a silica gel column, eluting with methylene chloride:methanol:NH₄OH 10:1.5:1 to afford the free base of the title compound. The base was converted to the salt by treatment with p-toluenesulfonic acid in ethanol to give the title compound (50 mg, 91%). mp. 65-67° C. MS (DCl/NH₃): m/z 255 (M+H)⁺. ¹H NMR (D₂O, 300 MHz) δ1.98 (m, 1H), 2.33 (m, 1H), 2.34 (s, 3H), 2.96 (m, 1H), 3.25-3.64 (m, 4H). 4.17-4.30 (m, 2H), 7.32 (d, J=8.1 Hz, 2H), 7.50-7.70 (m, 7H), 7.90 (brs, 1H), 8.28 (d, J=2.6 Hz, 1H), 8.50 (brs, 1H). Anal. Calc'd for C₁₆H₁₈N₂O.1.5 C₇H₈SO₃: C, 62.09; H, 5.90; N, 5.47. Found: C, 61.74; H, 6.23; N, 5.75.).

### EXAMPLE 29

### 3-(6-Methylpyridinyl-3-oxy)azepine dihydrochloride

### 29a. 1-Benzylpiperidine-2-methanol

2-Piperidinemethanol (3.45 g, 30 mmol) and benzaldehyde (4.53 g, 42.7 mmol) were heated to reflux in toluene (100 mL) for 4 hours. The volatiles were evaporated under reduced pressure and the residue was dissolved in acetic acid (100 mL). NaBH₃CN (2.3 g, 36 mmol) was added to the solution and the reaction mixture was stirred at room temperature for 16 hours. The solvents were evaporated and the residue was partitioned in NaHCO₃/ethyl acetate. The organic layer was separated, dried over MgSO₄ and evaporated. The residue was chromatographed on silica gel (eluent: 5% ethanol/methylene chloride saturated with NH₄OH) to yield 4.2 g (68%) of the title compound as an oil:

¹H NMR(CDCl₃, 300 MHz) δ1.45 (m, 1H), 1.62 (m, 2H), 1.78 (m, 3H), 2.45 (t, 1H), 2.83 (m, 1H), 3.04 (m, 1H), 3.71 (m, 2H), 3.98 (dd, 1H), 4.22 (d, 1H), 7.38 ppm (m, 5H); MS(DCl) m/z 206 (m+H)⁺.

### 29b. 1-Benzyl-2-chloromethylpiperidine

A solution of 1-benzylpiperidine-2-methanol (2.1 g, 10.5 mmol, from Example 29a) in methylene chloride (20 mL) was treated with 10.5 mL of 1 M HCl in ether. The solvents were evaporated and the resulting HCl salt was re-dissolved in CH₂Cl₂ (100 mL). Thionyl chloride (1.55 mL, 21 mmol) was added, and the solution was heated at 50° for 16 hours. The volatiles were evaporated and the residue was partitioned in NaHCO₃/CH₂Cl₂. The organic layer was separated, washed with brine, dried over MgSO₄ and evaporated to yield 1.6 g of the crude title compound as an oil:

¹H NMR(CDCl₃, 300 MHz) δ1.42 (m, 1H), 1.52 (m, 2H), 1.7 (m, 3H), 2.52 (m, 1H), 3.05 (m, 1H), 3.25 (m, 1H), 3.7 (m, 1H), 3.92 (m, 2H), 4.05 (m, 1H), 732 (m, 2H), 7.4 (m, 2H), 7.6 ppm (m, 1H).

### 29c. 1-Benzyl-3-(6-methylpyridinyl-3-oxy)perhydroazepine

A mixture of 1-benzyl-2-chloromethylpiperidine (0.3 g, 1.35 mmol, from Example 29b), 5-hydroxy-2-methylpyridine (0. 15 g, 1.35 mmol) and KOH (0.11 g, 1.5 mmol) was heated in DMF (2 mL) at 65° for 2 hours. The reaction mixture was then diluted with water and extracted with ethyl acetate. The organic layer was separated, washed with brine, dried over MgSO₄ and evaporated. The residue was chromatographed on silica gel (eluent: ethyl acetate/hexane (1:1)) to yield 0.22 g (55%) of the title compound as a white solid: ¹H NMR(CDCl₃, 300 MHz) δ1.7 (m, 4H), 2.12 (m, 2H), 2.42 (s, 3H), 2.6-2.95 (m, 4H), 3.68 (q, 2H), 4.3 (m, 1H), 6.65 (m, 1H), 6.88 (m, 1H), 7.32 (m, 5H), 8.05 ppm (d, 1H); MS(DCl) m/z 297 (m+H)⁺.

### 29d. 3-(6-Methylpyridinyl-3-oxy)perhydroazepine dihydrochloride

A solution of 1-benzyl-3-(5-methylpyridinyl-3-oxy)perhydroazepine (0.3 g, 1 mmol, from Example 29c) in methanol (10 mL) was stirred under H₂ (1 atm) for 48 hours in the presence of Pd/C (0.06 g). The catalyst was filtered off and the solvent was evaporated. The obtained residue was chromatographed on silica gel eluting with 10% methanol/methylene chloride saturated with NH₄OH to yield 0.08 g of the title compound that was converted to HCl salt: ¹H NMR (free base)(CDCl₃, 300 MHz) δ1.52 (m, 1H), 1.72 (m, 3H), 1.97 (m, 2H), 2.5 (s, 3H), 2.92 (m, 2H), 3.1 (m, 2H), 4.48 (m, 1H), 7.05 (d, 1H), 7.1 (dd, 1H) 8.18 (d, 1H); MS(DCI) m/z 207 (m+H)⁺. Anal. Calc'd for C₁₂H₁₇N₂O.2HCl: C, 51.88; H, 6.88; N, 10.07. Found: C, 51.40; H, 7.17; N, 9.87.

### EXAMPLE 30

### 3-(6-Fluoropyridinyl-3-oxy)perhydroazepine hydrochloride

The title compound was prepared according to the procedures of Examples 29c and 29d, substituting 2-fluoro-6-hydroxypyridine for the 2-methyl-5-hydroxypyridine therein. The product was obtained in 14% yield over two steps, including conversion to the HCl salt: mp 121-122° C.; ¹H NMR(free base)(CDCl₃, 300 MHz) δ1.52 (m, 2H), 1.75 (m, 2H), 1.95 (m, 2H), 3.05 (m, 2H), 3.2 (m, 2H), 4.51 (m, 1H), 6.82 (dd, 1H), 7.35 (m, 1H), 7.82 ppm (m, 1H); MS(DCl) m/z 211 (m+H)⁺; Anal. Calc'd for C₁₁H₁₅N₂OF. HCl: C, 53.55; H, 6.54; N, 11.35. Found: C, 53.35; H, 6.50; N, 11.08.

### EXAMPLE 31

### 6-Chloro-3-(3-(S)-pyrrolidinylmethoxy)pyridine

### 31 a. (S)-3-hydroxypyrrolidine

A mixture of (S)-(-)-1-benzyl-3-pyrrolidinol (15 g, 84.7 mmol, available as described in J. Med. Chem. 1986, 29, 2504-2511), 10% Pd-C (1.5 g,), and concentrated HCl (1 mL) in methanol (150 mL) was stirred under hydrogen (1 atm) at room temperature for 16 hours. The reaction mixture was filtered and concentrated to provide the title compound as an oil, suitable for use in the next step. MS (CI/NH₃) m/e 88 (M+H)⁺, 105 (M+NH₄)⁺.

### 31b. (1-(4-methylbenzenesulfonyl)-3-(S)-pyrrolidinyloxy) 4-methylbenzenesulfonate

A solution of the product from Example 31 a (84.7 mmol) in CH₂Cl₂ (250 mL) was treated with triethylamine (58.3 mL, 420 mmol) and p-toluenesulfonyl chloride (40.4 g, 212 mmol), and stirred at room temperature for 16 hours. The reaction mixture was diluted to 500 mL with CH₂Cl₂, then washed successively with water, 5% NaHCO₃, and brine. The organic layer was dried (MgSO₄), filtered and concentrated. The residue was flash chromatographed on silica gel with 30% ethyl acetate/hexane to provide the title compound as a white solid (26.1 g, 78%). MS (CI/NH₃) m/e 413 (M+NH₄)⁺; ¹H NMR (CDCl₃, 300 MHz) δ: 1.89-2.08 (m, 2H), 2.44 (s, 3H), 2.46 (s, 3H), 3.18-3.27 (m, 1H), 3.33-3.37 (m, 1H), 3.41-3.51 (m, 2H), 4.92-4.97 (m, 1H), 7.29-7.36 (m, 4H), 7.64-7.69 (m, 4H).

### 31 c. 1-(4-methylbenzenesulfonyl)-3-(R)-pyrrolidinenitrile

A solution of the product from Example 31b (15.2 g, 38.5 mmol) in DMF (150 mL) was treated with sodium cyanide (2.89 g, 57.8 mmol) and water (25 mL), and stirred at 100° C. for 16 hours. The bulk of the solvent was removed under reduced pressure at 60° C., and the residue was partitioned with H₂O (80 mL) and CH₂Cl₂ (300 mL). The organic layer was washed with water and brine, then dried (MgSO₄), filtered and concentrated. The residue was purified on silica gel (40% ethyl acetate/hexane) to provide the title compound as a white solid (4.86 g, 50%). MS (CI/NH₃) m/e 251 (M+H)⁺, 268 (M+NH₄)⁺; ¹H NMR (CDCl₃, 300 MHz) δ: 2.02-2.13 (m, 1H), 2.18-2.29 (m, 1H), 2.46 (s, 3H), 2.95-3.04 (m, 1H), 3.31-3.47 (m, 3H), 3.65-3.71 (dd, J=7, 11 Hz, 1H), 7.36 (d, J=8 Hz, 2H), 7.73 (d, J=8 Hz, 2H).

### 31d. 1-(4-methylbenzenesulfonyl)pyrrolidine-3-carboxylic acid

A solution of the product from Example 31c (4.86g, 19.4 mmol) in ethylene glycol (50 mL) was treated with KOH (50 mL of 40% aqueous solution). The reaction mixture was stirred at 130° C. for 3.5 hours, then cooled to room temperature. The reaction mixture was diluted with water (50 mL) and acidified with concentrated HCl. The mixture was extracted with ethyl acetate (3×100 mL). The combined organic extract was washed with water and brine, dried (MgSO₄), filtered and concentrated to provide the crude title compound as a white solid (4.98 g, 95%). MS (CI/NH₃) m/e 270 (M+H)⁺, 287 (M+NH₄)⁺; ¹H NMR (CDCl₃, 300 MHz) δ: 2.03-2.12 (m, 2H), 2.44 (s, 3H), 2.95-3.04 (m, 1H), 3.33-3.35 (m, 2H), 3.41-3.46 (dd, J=5, 11 Hz, 1H), 3.52-3.58 (dd, J=8, 11 Hz, 1H)), 7.33 (d, J=8 Hz, 2H), 7.72 (d, J=8 Hz, 2H).

### 31e. 1-(4-methylbenzenesulfonyl)pyrrolidine-3-methanol

A solution of the product from Example 31d (4.98 g, 18.5 mmol) in anhydrous THF (30 mL) was cooled to 0° C. and treated with borane (1M solution in THF, 27.8 mL), added over 30 minutes. The ice bath was then removed and the reaction mixture was stirred at room temperature for 2 hours. HCl (1N aqueous solution, 20 mL) was added slowly to quench the reaction. The resulting solution was then stirred overnight. The volatiles were removed under reduced pressure, and the remaining aqueous phase was extracted with dichloromethane (4×80 mL). The combined dichloromethane extract was washed with brine, dried (MgSO₄), filtered, and concentrated. The residue was purified on silica gel (40% ethyl acetate/hexane) to provide the title compound as a white solid (4.33 g, 92%). MS (CI/NH₃) m/e 256 (M+H)⁺, 273 (M+NH₄)⁺; ¹H NMR (CDCl₃, 300 MHz) δ: 1.36 (t, J=5 Hz, 1H), 1.56-1.64 (m, 1H), 1.86-1.97 (m, 1H), 2.28-2.37 (m, 1H), 2.44 (s, 3H), 3.04-3.10 (dd, J=6, 10 Hz, 1H), 3.16-3.23 (m, 1H), 3.29-3.38 (m, 2H), 3.50-3.63 (m, 2H), 7.33 (d, J=8 Hz, 2H), 7.72 (d, J=8 Hz, 2H).

### 31f. 1-(4-methylbenzenesulfonyl)pyrrolidine-3-methyl 4-methyl benzenesulfonate

A mixture of the product from Example 31e (4.33 g, 17.0 mmol) in CH₂Cl₂ (50 mL) was treated with triethylamine (7.1 mL, 51.0 mmol) and p-toluenesulfonyl chloride (4.86 g, 25.5 mmol) at room temperature for 16 hours. The reaction mixture was diluted with CH₂Cl₂ (150 mL), then washed successively with water, 5% NaHCO₃, and brine. The organic phase was dried (MgSO₄), filtered and concentrated. The residue was purified on silica gel (30% ethyl acetate/hexane) to provide the title compound as a light yellow solid (6.40 g, 92%). MS (CI/NH₃) m/e 410 (M+H)⁺, 427 (M+NH₄)⁺; ¹H NMR (CDCl₃, 300 MHz) δ: 1.48-1.53 (m, 2H), 1.84-1.94 (m, 1H), 2.45 (s, 3H), 2.47 (s, 3H), 2.92-2.98 (dd, J=6, 10 Hz, 1H), 3.08-3.17 (m, 1H), 3.23-3.33 (m, 2H), 3.77-3.89 (m, 2H), 7.31-7.39 (m, 4H), 7.68 (d, J=8 Hz, 2H), 7.74 (d, J=8 Hz, 2H).

### 31g. 2-Chloro-5-(1-(4-methylbenzenesulfonyl)pyrrolidinyl-3-methoxy)pyridine

A solution of the product from Example 3f (892 mg, 2.18 mmol) in DMF (20 mL) was combined with potassium hydroxide (305 mg, 5.45 mmol) and 2-chloro-5-hydroxypyridine (353 mg, 2.73 mmol), and stirred at 85° C. for 12 hours. The bulk of the solvent was removed under reduced pressure at 60° C., and the residue was dissolved in a mixture of H₂O (10 mL) and CH₂Cl₂ (100 mL). The organic layer was washed with water and brine, then dried (MgSO₄), filtered and concentrated. The residue was purified on silica gel with 20% ethyl acetate/hexane to provide the title compound as a light yellow solid (710 mg, 89%). MS (CI/NH₃) m/e 367 (M+H)⁺, 384 (M+NH₄)⁺; ¹H NMR (CDCl₃, 300 MHz) δ: 1.65-1.77 (m, 1H), 1.98-2.09 (m, 1H), 2.42 (s, 3H), 2.56-2.65 (m, 1H), 3.19-3.24 (dd, J=6, 11 Hz, 1H), 3.25-3.38 (m, 2H), 3.40-3.46 (dd, J=7, 10 Hz, 1H), 3.62 (t, J=9 Hz, 1H), 3.75-3.80 (dd, J=6, 9 Hz, 1H), 7.04-7.8 (dd, J=3, 8 Hz, 1H), 7.22 (d, J=9 Hz, 1H), 7.29 (d, J=8 Hz, 2H), 7.72 (d, J=8 Hz, 2H), 7.90 (d, J=3 Hz, 1H).

### 31h. 2-Chloro-5-(pyrrolidinyl-3-methoxy)pyridine

A mixture of the product from Example 31g (710 mg, 1.94 mmol) in HBr/HOAc (10 mL, 30 wt. % in acetic acid) was stirred at room temperature for 16 hours. Excess reagent was removed under reduced pressure, and the residue was dissolved in dichloromethane (80 mL) and made basic with saturated ammonium hydroxide (10 mL). The aqueous phase was extracted with dichloromethane (3×30 mL). The combined dichloromethane extract was washed with brine, then dried (MgSO₄), filtered and concentrated. The residue was purified on silica gel (90/10/1 CH₂Cl₂/MeOH/NH₄OH) to provide the title compound as a light yellow oil (78 mg, 19%). MS(CI/NH₃) m/e 213 (M+H)⁺, 230 (M+NH₄)⁺; ¹HNMR(CDCl₃, 300 MHz) δ: 1.51-1.62 (m, 1H), 1.94-2.05 (m, 1H), 2.53-2.63 (m, 1H), 2.79-2.85 (d, J=6, 11 Hz, 1H), 2.94-3.06 (m, 2H), 3.12-3.18 (dd, J=8, 11 Hz, 1H), 3.86-3.98 (m, 2H), 7.17 (d, J=3 Hz, 1H), 7.25 (d, J=4 Hz, 1H), 8.05 (s, 1H).

### EXAMPLE 32

### 2-Chloro-5-(1-methylpyrrolidinyl-3-methoxy)pyridine p-toluenesulfonate

### 32a. 6-Chloro-3-(1-methylpyrrolidinyl-3-methoxy)pyridine

A solution of the product from Example 31h (78 mg, 0.368 mmol) in a mixture of formaldehyde (37 wt. % in water, 7 mL) and formic acid (4 mL) was stirred at 65° C. for 16 hours. The volatiles were removed under reduced pressure at 45° C. The residue was taken up in aqueous 1N NaOH (5 mL) and extracted with CH₂Cl₂ (3×30 mL). The combined CH₂Cl₂ extract was washed with brine, dried (MgSO₄), filtered, and concentrated. The residue was purified on silica gel (95/5/0.5 CH₂Cl₂/MeOH/NH₄OH) to provide the title compound a light yellow oil (53%, 44 mg). MS (CI/NH₃) m/e 227 (M+H)⁺.

### 32b. 2-Chloro-5-(1-methylpyrrolidinyl-3-methoxy)pyridine p-toluenesulfonate

A solution of the product from Example 32a (39 mg, 0.185 mmol) in ethyl acetate (1 mL) was treated with p-toluenesulfonic acid monohydrate (37 mg, 0.194 mmol) at room temperature for 5 minutes. Ethyl ether (30 mL) was added and the mixture was stirred for 5 minutes longer. The ether was decanted and the trituration procedure was repeated once more. The solid was dried under vacuum to provide the title compound. mp 75-77° C.; MS (ESI⁺) m/e 227 (M+H)⁺; ¹H NMR (D₂O, 400 MHz) δ: 1.93-2.15 (m, 1H), 2.25-2.45 (m, 1H), 2.49 (s, 3H), 2.98 (s, 3H), 2.98-3.12 (m, 1H), 3.15-3.30 (m, 1H), 3.30-3.45 (m, 1H), 3.58-3.95 (m, 2H), 4.05-4.19 (m, 2H), 7.35 (d, J=8 Hz, 2H), 7.41 (d, J=9 Hz, 1H), 7.44-7.47 (dd, J=3, 9 Hz, 1H), 7.69 (d, J=8 Hz, 2H), 8.05 (d, J=3 Hz, 1H); Analysis calculated for C₁₁H₁₅N₂ClO.C₇H₈O₃S: C, 54.20; H, 5.81; N, 7.02; Found: C, 53.98; H, 5.78; N, 6.79.

### EXAMPLE 33

### 2,3-Dichloro-5-(pyrrolidinyl-3-methoxy)pyridine p-toluenesulfonate

### 33a. 2,3-Dichloro-5-(1-(4-methylbenzenesulfonyl)pyrrolidinyl-3-methoxy)pyridine

A solution of the product from Example 31f (957 mg, 2.34 mmol) in DMF (20 mL) was treated with potassium hydroxide (289 mg, 5.15 mmol) and 2,3-dichloro-5-hydroxyl pyridine (384 mg, 2.34 mmol) at 85° C. for 16 hours. The bulk of the DMF was removed under vacuum at 60° C. The residue was dissolved in a mixture of H₂O (10 mL) and CH₂Cl₂ (100 mL). The organic layer was washed with water and brine, then dried (MgSO₄), filtered and concentrated. The residue was purified on silica gel (20% ethyl acetate/hexane) to provide the title compound as a white solid (672 mg, 72%). MS (CI/NH₃) m/e 401 (M+H)⁺, 418 (M+NH₄)⁺; ¹H NMR (CDCl₃, 300 MHz) δ: 1.64-1.77 (m, 1H), 1.98-2.11 (m, 1H), 2.42 (s, 3H), 2.56-2.66 (m, 1H), 3.18-3.23 (dd, J=6, 10 Hz, 1H), 3.26-3.38 (m, 2H), 3.40-3.46 (dd, J=7, 10 Hz, 1H), 3.62 (t, J=10 Hz, 1H), 3.75-3.80 (dd, J=6, 9 Hz, 1H), 7.20 (d, J=3 Hz, 1H), 7.29 (d, J=8 Hz, 2H), 7.72 (d, J=8 Hz, 2H), 7.86 (d, J=3 Hz, 1H).

### 33b. 2,3-Dichloro-5-(pyrrolidinyl-3-methoxy)pyridine

The product from Example 33a (673 mg, 1.68 mmol) was treated according to the procedure of Example 31h to provide the title compound as a plae yellow oil (54 mg, 13%). MS(CI/NH₃) m/e 247 (M+H)⁺.

### 33c. 2,3-Dichloro-5-(pyrrolidinyl-3-methoxy)pyridine p-toluenesulfonate

The product of Example 33b (50 mg, 0.20 mmol) was combined with p-toluenesulfonic acid monohydrate (41 mg, 0.21 mmol) according to the procedure of Example 32b to provide the salt as a white solid. mp 82-84° C.; MS (ESI⁺) m/e 247 (M+H)⁺; ¹H NMR (D₂O, 500 MHz) δ: 1.93-2.01 (m, 1H), 2.26-2.33 (m, 1H), 2.39 (s, 3H), 2.90-2.96 (m, 1H), 3.24-3.28 (dd, J=7, 12 Hz, 1H), 3.35-3.40 (m, 1H), 3.46-3.51 (m, 1H), 3.56-3.60 (dd, J=8, 12 Hz, 1H), 4.10-4.14 (dd, J=6, 10 Hz, 1H), 4.19-4.22 (dd, J=5, 10 Hz, 1H), 7.69 (d, J=2 Hz, 1H), 7.70 (d, J=5 Hz, 2H), 8.04 (d, J=3 Hz, 1H); Analysis calculated for C₁₀H₁₂N₂Cl₂O.1.15C₇H₈O₃S.0.25H₂O: C, 48.21; H, 4.86; N, 6.23; Found: C, 47.98; H, 4.77; N, 6.24.

### EXAMPLE 34

### 2-Chloro-3-methyl-5-(3-(R)-pyrrolidinylmethoxy)pyridine

### 34a. 3-(R)-Pyrrolidinemethanol

A mixture of (3R)-1-[(R)-1-phenethyl]-3-(hydroxymethyl)pyrrolidine (829 mg, 4.04 mmol, prepared as described in J. Med. Chem., 1990, 33, 71-77), palladium (83 mg, 20 wt. % on activated carbon), concentrated HCl (1 mL) in methanol (15 mL) at room temperature was hydrogenated at 4 atmosphere for 6 days. The reaction mixture was filtered and concentrated to provide the title compound as an oil, suitable for use in the next step. MS (CI/NH₃) m/e 102 (M+H)⁺, 119 (M+NH₄)⁺.

### 34b. 1-(BOC)-3-(R)-pyrrolidinemethanol

A solution of the product from Example 34a (352 mg, 3.49 mmol) in dichloromethane (10 mL) was treated with triethylamine (1.45 mL, 10.5 mmol) and di-tert-butyldicarbonate (1.13 g, 5.23 mmol) and stirred at room temperature for 16 hours. The volatiles were removed under reduced pressure, and the residue was dissolved in dichloromethane (50 mL), and washed with saturated ammonium chloride, saturated sodium carbonate and brine. The organic phase was dried over anhydrous MgSO₄, concentrated and the residue was purified on silica gel (40% ethyl acetate/hexane) to provide the title compound as a light yellow oil (146 mg, 21%). MS (CI/NH₃) m/e 202 (M+H)⁺, 219 (M+NH₄)⁺; ¹HNMR (CDCl₃, 300 MHz) δ: 1.46 (s, 9H), 1.62-1.72 (m, 1H), 1.92-2.03 (m, 1H), 2.35-2.46 (m, 1H), 3.05-3.15 (m, 1H), 3.28-3.38 (m, 1H), 3.45-3.55 (m, 2H), 3.57-3.67 (m, 2H).

### 34c. 1-(BOC)-3-(R)-pyrrolidinemethyl 4-methylbenzenesulfonate

A mixture of the product from Example 34b (140 mg, 0.697 mmol) in CH₂Cl₂ (5 mL) was treated with triethylamine (0.24 mL, 1.74 mmol) and p-toluenesulfonyl chloride (166 mg, 0.871 mmol) at room temperature for 16 hours. The reaction mixture was diluted with CH₂Cl₂ (200 mL), then washed with water, 5% NaHCO₃, and brine. The organic phase was dried (MgSO₄), filtered and concentrated. The residue purified on silica gel (30% ethyl acetate/hexane) to provide the title compound as a white solid (160 mg, 65%). MS (CI/NH₃) m/e 356 (M+H)⁺, 373 (M+NH₄)⁺; ¹H NMR (CDCl₃, 300 MHz) δ: 1.44 (s, 9H), 1.65-1.75 (m, 1H), 1.90-2.00 (m, 1H), 2.46 (s, 3H), 2.48-2.57 (m, 1H), 2.97-3.03 (dd, J=7, 11 Hz, 1H), 3.08-3.17 (m, 1H), 3.23-3.40 (m, 2H), 3.42-3.50 (m, 1H), 3.90-4.03 (m, 2H), 7.36 (d, J=8 Hz, 2H), 7.79 (d, J=8 Hz, 2H).

### 34d. 2-Chloro-3-methyl-5-(1-(BOC)-3-(R)-pyrrolidinylmethoxy)pyridine

A solution of the product from Example 34c(155 mg, 0.437 mmol) in DMF (6 mL) was treated with potassium hydroxide (61 mg, 1.09 mmol) and 2-chloro-3-methyl-5-hydroxyl pyridine (78 mg, 0.546 mmol), and stirred at 85° C. for 16 hours. DMF was removed under reduced pressure at 60° C. The residue was dissolved in a mixture of H₂O (10 mL) and CH₂Cl₂ (100 mL). The organic layer was washed with water and brine, then dried (MgSO₄), filtered and concentrated. The residue was purified on silica gel with 20% ethyl acetate/hexane to provide the title compound as a white solid (124 mg, 87%).

MS (CI/NH₃) m/c 327 (M+H)⁺, 344 (M+NH₄)⁺;

¹H NMR (CDCl₃, 300 MHz) δ: 1.47 (s, 9H), 1.70-1.84 (m, 1H), 2.01-2.13 (m, 1H), 2.35 (s, 3H), 2.61-2.72 (m, 1H), 3.13-3.27 (m, 1H), 3.30-3.43 (m, 2H), 3.55-3.65 (m, 1H), 3.90-3.98 (m, 2H), 7.10 (d, J=2 Hz, 1H), 7.90 (d, J=3 Hz, 1H).

### 34e. 2-Chloro-3-methyl-5-(3-(R)-pyrrolidinylmethoxy)pyridine 4-methylbenzenesulfonate

A solution of the product from Example 34d (117 mg, 0.359 mmol) in CH₂Cl₂ (5 mL) was treated with p-toluenesulfonic acid monohydrate (72 mg, 0.377 mmol) and heated at reflux for 10 hours. The solvent was removed in a stream of nitrogen, and the residue was triturated with ether (2×30 mL) to provide the title compound as a white solid (120 mg, 83%).

mp 102-104° C.; MS (ESI⁺) m/e 227 (M+H)⁺; ¹H NMR (D₂O, 500 MHz) δ: 1.92-1.99 (m, 1H), 2.25-2.32 (m, 1H), 2.35 (s, 3H), 2.39 (s, 3H), 2.90-2.94 (m, 1H), 3.23-3.27 (dd, J=7, 12 Hz, 1H), 3.34-3.39 (m, 1H), 3.45-3.50 (m, 1H), 3.54-3.59 (dd, J=8, 12 Hz, 1H), 4.07-4.11(dd, J=7, 10 Hz, 1H), 4.16-4.19 (dd, J=5, 9 Hz, 1H), 7.37 (d, J=8 Hz, 2H), 7.42 (d, J=3 Hz, 1H), 7.69 (d, J=9 Hz, 2H), 7.91 (d, J=3 Hz, 1H); Analysis calculated for C₁₁H₁₅N₂ClO.1.45C₇H₈O₃S.0.45H₂O: C, 52.43; H, 5.72; N, 5.78; Found C, 52.11; H, 5.84; N, 5.88. [α]²⁵_{D}=+0.60° (c=0.50, MeOH).

### EXAMPLE 35

### 3-Bromo-2-chloro-5-(3-(R)-pyrrolidinylmethoxy)pyridine 4-methyl benzenesulfonate

### 35a. 3-Bromo-2-chloro-5-(1-[BOC]3-(R)-pyrrolidinylmethoxy)pyridine

This compound was prepared according to the procedure of Example 34d, substituting 3-bromo-2-chloro-5-hydroxypyridine for the 2-chloro-3-methyl-5-hydroxypyridine therein. The product was obtained as a yellow oil in 86% yield. MS (CI/NH₃) m/e 391 (M+H)⁺, 408 (M+NH₄)⁺;

¹H NMR (CDCl₃, 300 MHz) δ: 1.47 (s, 9H), 1.70-1.87 (m, 1H), 2.02-2.15 (m, 1H), 2.62-2.73 (m, 1H), 3.13-3.27 (m, 1H), 3.30-3.53 (m, 2H), 3.55-3.65 (m, 1H), 3.90-3.98 (m, 2H), 7.50 (d, J=3 Hz, 1H), 8.03 (d, J=3 Hz, 1H).

### 35b. 5-Bromo-6-chloro-3-(3-(R)-pyrrolidinylmethoxy)pyridine 4-methylbenzenesulfonate

The product of Example 35a was deprotected and converted to the salt according to the procedure of Example 34e, to provide the title compound in 77% yield. MS (DCI-NH₃)m/z 308 (M+1)⁺; ¹H NMR (300 MHz, MeOH-d₄) δ: 1.8-2.0 (m, 1H), 2.2-2.4 (m, 1H), 2.8-3.0 (m, 1H), 3.1-3.6 (m, 4H), 4.05-4.25 (m, 2H), 7.22 (d, J=9 Hz, 2H), 7.70 (d, J=9 Hz, 2H), 7.82 (d, J=3 Hz, 1H), 8.1 (d, J=3 Hz, 1H); Analysis calculated for C₁₀H₁₂BrClN₂O.C₇H₈O₃S: C, 44.00; H, 4.31; N, 6.04. Found: C, 44.24; H, 4.22; N, 6.10.

### EXAMPLE 36

### 36a. 3-Bromo-2-chloro-5-(1-methyl-3-(R)-pyrrolidinylmethoxy)pyridine

A solution of the product from Example 35a (100 mg, 0.256 mmol) in a mixture of formaldehyde (37 wt. % in water, 7 mL) and formic acid (4 mL) was stirred at 65° C. for 16 hours. The excess reagents were removed under reduced pressure at 45° C. The residue was taken up in aqueous 1N NaOH solution (10 mL) and extracted with CH₂Cl₂ (3×30 mL). The combined CH₂Cl₂ extract was washed with brine, dried (MgSO₄), filtered, and concentrated. The residue was purified on silica gel (95/5/0.5 CH₂Cl₂/MeOH/NH₄OH) to provide a light yellow oil (46 mg, 59%). MS (CI/NH₃) m/e 305 (M+H)⁺.

### 36b. 3-Bromo-2-chloro-5-(3-(R)-pyrrolidinylmethoxy)pyridine 4-methylbenzenesulfonate

The product from Example 36a (46 mg, 0.151 mmol)was converted to the toluenesulfonate salt according to the procedure of Example 32b to provide the title compound as a white hygroscopic solid;

MS (ESI⁺) m/e 305 (M+H)⁺; ¹H NMR (D₂O, 500 MHz) δ: 2.01-2.09 (m, 1H), 2.33-2.43 (m, 1H), 2.38 (s, 3H), 2.98 (s, 3H), 2.98-3.08 (m, 1H), 3.30-3.70 (m, 4H), 4.06-4.09 (dd, J=6, 10 Hz, 2H), 4.12-4.15 (dd, J=5, 9 Hz, 2H), 7.33 (d, J=8 Hz, 2H), 7.68 (d, J=8 Hz, 2H), 7.75 (d, J=2 Hz, 1H), 8.01 (d, J=2 Hz, 1H);

Analysis calculated for C₁₁H₁₄N₂BrClO.C₇H₈O₃S.0.45H₂O: C, 44.49; H, 4.75; N, 5.77; Found: C, 44.43; H, 4.75; N, 5.54. [α]²⁵D=−2.9° (c=3.4, MeOH).

### EXAMPLE 37

### 2-Chloro-5-(3-(R)-pyrrolidinylmethoxy)pyridine 4-methylbenzenesulfonate

This compound was prepared according to the procedure of Examples 34d and 34e, substituting 2-chloro-5-hydroxypyridine for the 2-chloro-3-methyl-5-hydroxypyridine therein. The product was obtained as a white solid.

mp 137-139° C.; MS (CI/NH₃) m/e 213 (M+H)⁺; ¹H NMR (D₂O, 400 MHz) δ: 1.92-2.02 (m, 1H), 2.26-2.34 (m, 1H), 2.40 (s, 3H), 2.89-2.96 (m, 1H), 3.24-3.29 (dd, J=7, 12 Hz, 1H), 3.34-3.41 (m, 1H), 3.46-3.52 (m, 1H), 3.55-3.60 (dd, J=8, 12 Hz, 1H), 4.10-4.14 (dd, J=6, 9 Hz, 1H), 4.18-4.22 (dd, J=6, 10 Hz, 1H), 7.37 (d, J=8 Hz, 2H), 7.43 (d, J=9 Hz, 1H), 7.47-7.50 (dd, J=3, 9 Hz, 1H), 7.69 (d, J=8 Hz, 2H), 8.07 (d, J=3 Hz, 1H); Analysis calculated for C₁₀H₁₃N₂ClO.C₇H₈O₃S: C, 53.05; H, 5.50; N, 7.28; Found: C, 52.91; H, 5.36; N, 7.16. [α]²⁵_{D}=+1.45° (c=0.55, MeOH).

### EXAMPLE 38

### 2-Fluoro-5-(3-(R)-pyrrolidinylmethoxy)pyridine 4-methylbenzenesulfonate

This compound was prepared according to the procedure of Examples 34d and 34e, substituting 2-fluoro-5-hydroxypyridine (NCK Chemicals, Denmark) for the 2-chloro-3-methyl-5-hydroxypyridine therein. The product was obtained as a white solid. mp 108-110° C.; MS (CI/NH₃) m/z 197 (M+H)⁺; ¹HNMR (CDCl₃, 300 MHz): δ1.93 (m, 1H), 2.20 (m, 1H), 2.36 (s, 3H), 2.82 (m, 1H), 3.37 (m, 2H), 3.53 (m, 2H), 3.94 (d, J=6 Hz, 2H), 6.74 (dd, J=9, 3 Hz, 1H), 7.16 (d, J=8 Hz, 2H), 7.24 (ddd, J=9, 6, 3 Hz, 1H), 7.69 (d, J=8 Hz, 2H), 7.73 (m, 1H).

### EXAMPLE 39

### 2-Chloro-5-(3-(S)-pyrrolidinylmethoxy)pyridine 4-methylbenzenesulfonate

### 39a. 1-(BOC)-3-(S)-pyrrolidinemethanol

This compound was prepared by analogy to the procedures in Examples 34a and b, starting with (3S)-1-[(R)-1-phenethyl]-3-(hydroxymethyl)pyrrolidine (prepared as described in J. Med. Chem., 1990, 33, 71-77), to afford the title compound in 64% yield. MS (DCI-NH₃) m/z 202 (M+H)⁺; ¹H NMR (300 MHz, CDCl3) δ1.56 (s, 9H), 1.6-1.7 (m, 1 H),1.9-2.05 (m, 1H), 2.35-2.45 (m, 1 H), 3.05-3.15 (m, 1 H), 3.25-3.75 (m, 5H).

### 39b. 1-(BOC)-3-(S)-pyrrolidinemethyl methanesulfonate

The alcohol from Example 39a (450 mg, 2.23 mmol) was dissolved in dry THF (10 mL) and cooled to 0-5° C. Triethylamine (225.8 mg, 2.23mmol) was added, followed by methanesulfonyl chloride (256mg, 2.23mmol). The solution was allowed to warm to room temperature for 5 hours, then was partitioned with brine (30 mL) and ethyl acetate (50 mL). The organic layer was separated, dried (MgSO₄) and under vacuum to leave the crude title compound (575 mg, 87%) as an oil, suitable for use in the next reaction. MS (DCI-NH₃) m/z 297 (M+); ¹H NMR (300 MHz, CDCl₃) δ1.56 (s, 9H), 1.7-1.8 (m, 1H),1.95-2.1 (m, 1H), 2.55-2.70 (m, 1H),3.4 (s, 3H), 3.05-3.20 (m, 1 H), 3.30-3.65 (m, 3H), 4.1-4.3(m, 2H).

### 39c. 2-Chloro-5-(1-(BOC)-3-pyrrolidinylmethoxy)pyridine

2-Chloro-5-hydroxypyridine (272 mg, 1.93 mmol) was dissolved in dry DMF (5 ml) and cooled in an ice bath. Cesium carbonate (1.6 mg, 5 mmol) and product of Example 39b (540 mg, 1.93 mmol) were added and the mixture was warmed to 90° C. for 2.5 hours. The reaction mixture was quenched with brine and extracted with ethyl acetate. The organic layer was separated, dried (MgSO₄), and concentrated under vacuum. The compound was purified on silica gel column (80:20 pentane-ethyl acetate) to provide the title compound (330 mg, 55%). MS (DCI-NH₃) m/z 313 (M +H)⁺, ¹H NMR (300 MHz, CDCl₃) δ1.45 (s, 9H), 1.70-1.85 (m, 1H), 2.05-2.15 (m, 1H), 2.5-2.75(m, 1H), 3.12-3.28 (m, 1H), 3.30-3.55 (m, 2 H), 3.55-3.65 (m, 1H), 3.90-4.0 (m, 2H),7.15,(dd, J=9 Hz, 3 Hz, 1H ),7.25(d, J=9 Hz, 1H), 8.05 (d, J=3 Hz, 2 H).

### 39d. 2-Chloro-5-(3-(S)-pyrrolidinylmethoxy)pyridine 4-methylbenzenesulfonate

The product of Example 39c (330 mg, 1.05 mmol) was heated to reflux with p-toluenesulfonic acid (200 mg, 1.05 mmol) in ethyl acetate (50ml). After 2.5 hours, the mixture was cooled to afford white crystals of the title salt (188 mg, 47%). mp 144-145° C. MS (DCI-NH₃) m/z 213 (M +H)⁺;

¹H NMR (300 MHz, CDCl₃) δ1.84-2.0 (m, 1H), 2.14-2.30 (m, 1 H),2.36 (s, 3H), 2.76-2.92 (m, 1H), 3.30-3.45 (m, 2 H), 3.50-3.6 (m, 2H), 3.95 (d, J=6 Hz, 2H), 7.1 (m, 1H), 7.5 (d, J=7.5 Hz, 2H), 7.68 (d, J=7.5 Hz, 2 H), 7.96 (d, J=3 Hz, 1H), 9.25(bs, 1H); Analysis calculated for C₁₀H₁₃ClN₂O.C₇H₈O₃S: C, 53.05; H, 5.46; N, 7.28. Found: C, 52.79; H, 5.47; N, 7.26; [α]_{D}²³−1.49° (c 0.69, MeOH).

### EXAMPLE 40

### 2-Fluoro-5-(3-(S)-pyrrolidinylmethoxy)pyridine 4-methylbenzenesulfonate

The title compound was prepared according to the procedures of Examples 39c and d, using 2-fluoro-5-hydroxypyridine (NCK Chemicals, Denmark) in place of the 2-chloro-5-hydroxypyridine therein. mp 109-110° C.; MS (DCI-NH₃) m/z 197 (M +H)⁺; ¹H NMR (300 MHz, CDCl₃) δ1.85-2.0 (m, 1 H), 2.15-2.30 (m, 1 H), 2.36 (s, 3H), 2.75-2.92 (m, 1 H), 3.30-3.45 (m, 2 H), 3.42-3.62 (m, 2H), 3.95 (d, J=6 Hz, 2H), 6.75 (dd, J=9 Hz,Hz, 1H), 7.16 (d, J=7.5 Hz, 2H), 7.25 (dd, J=9 Hz 6 Hz, 1 H), 7.65-7.75 (m, 3H), 9.25 (bs, 1H); Analysis calculated for C₁₀H₁₃FN₂O.C₇H₈O₃S.0.25 H₂O: C, 54.75; H, 5.81; N, 7.51. Found: C, 54.85; H, 5.56; N, 7.59; [α]_{D}²³ −4.65° (c 0.2, MeOH).

### EXAMPLE 41

### 3-Bromo-2-chloro-5-(3-(S)-pyrrolidinylmethoxy)pyridine 4-methyl benzenesulfonate

### 41a. 3-Bromo-2-chloro-5-(1-[BOC]-3-(S)-pyrrolidinylmethoxy)pyridine

Condensation of 3-bromo-2-chloro-5-hydroxypyridine with 1-(BOC)-3-(S)-pyrrolidinemethyl methanesulfonate from Example 39b, according to the procedure of Example 39c, provided the title compound in 85% yield after chromatography on silica gel (5% EtOAc-pentane). MS (DCI-NH₃)m/z 408 (M+1)⁺; ¹HNMR (300 MHz, CDCl₃) δ1.46 (s, 9H), 1.7-1.9 (m, 1H), 2.05-2.2 (m, 1H), 2.60-2.76 (m, 1H), 3.12-3.65 (m, 4H), 3.95-4.15 (m, 2H), 7.8 (d, J=3 Hz, 1H) 8.08 (d, J=3 Hz, 1H).

### 41b. 3-Bromo-2-chloro-5-(3-(S)-pyrrolidinylmethoxy)pyridine 4-methylbenzenesulfonate

The product of Example 41a was carried through the procedure of Example 39d to provide the salt as a white crystalline solid in 58% yield.MS (DCI-NH₃) m/z 291/293/295 (M+H)⁺; ¹H NMR (300 MHz, MeOH-d₄) δ1.85-2.0 (m, 1H), 2.2-2.35 (m, 1H), 2.8-2.95 (m, 1H), 3.1-3.6 (m, 4H), 4.05-4.25 (m, -2H), 7.22 (d, J=9 Hz, 2H)7.7 (d, J=9 Hz, 2H), 7.82 (d, J=3Hz, 1H), 8.1 (d,J=3 Hz, 1H); Calculated analysis for C₁₀H₁₂BrClN₂O.C₇H₈O₃S: C, 44.03; H, 4.35; N, 6.04. Found: C, 44.21; H, 4.38; N, 5.90.

### EXAMPLE 42

### 2-chloro-5-(3-(S)-pyrrolidinylmethoxy)-3-pyridinecarboxaldehyde hydrochloride

### 42a. 2-Chloro-5-(1-[BOC]-3-(S)-pyrrolidinylmethoxy)-3-pyridinecarboxaldehyde

A solution of the product of Example 41a (275 mg, 0.67 mmol) in anhydrous THF (100 mL) and DMF (0.21 mL, 2.7 mM) was cooled to −78° C. under N₂. n-BuLi (2.5 M in hexanes, 0.86 mL, 2.16 mM) was added and the yellow solution was stirred for 20 minutes at −78° C., then allowed to warm to −20° C. The reaction was quenched with brine (50 mL) and extracted with EtOAc (3×150 mL). The extract was dried (MgSO₄), filtered and concentrated in vacuo. The residue was purified on silica gel (2-10% EtOAc-pentane) to provide the title compound (235 mg, 37%). MS (DCI-NH₃) m/z 358 (M+NH₄)⁺;

¹H NMR (300 MHz, CDCl₃) δ1.7-1.9 (m, 1H), 2.05-2.2 (m, 1H), 2.6-2.8 (m, 1H), 3.15-3.7(m, 4H), 3.9-4.1 (m, 2H), 7.08 (d, J=3 Hz, 1H) 8.31 (d, J=3 Hz, 1H).

### 42b. 2-Chloro-5-(3-(S)-pyrrolidinylmethoxy)-3-pyridinecarboxaldehyde hydrochloride

The product of Example 42a (190 mg, 0.53 mM) was treated with 4N HCl in dioxane (0.8 mL) and stirred at room temperature for 1 hr. A white precipitate appeared. The solvent was removed under vacuum, and the residue was dissolved in hot EtOH (3 mL). Ethyl acetate (6 mL) was added, and the solution was allowed to cool slowly to room temperature, and finally in the refrigerator overnight. The pale yellow solid was filtered and washed with EtOAc, then dried to yield 38 mg (28%) of the title compound.

MS (DCI-NH₃) m/z 241 (M+H)⁺; ¹H NMR (300 MHz, MeOH-d₄) δ1.9-2.05 (m, 1H), 2.2-2.35 (m, 1H), 2.8-3.0 (m, 1H), 3.15-3.36 (m, 4H), 4.05-4.25 (m, 2H), 7.68 (d, J=3 Hz, 1H) 8.05 (d, J=3 Hz, 1H); Calculated analysis for C₁₁H₁₃ClN₂O₂.HCl: C, 47.67; H, 5.09; N, 10.11. Found C, 47.67; H, 4.88; N, 10.15.

### EXAMPLE 43

### 2-Chloro-3-hydroxymethyl-5-(3-(S)-pyrrolidinylmethoxy)pyridine hydrochloride

### 43a. 2-Chloro-5-(1-[BOC]-3-(S)-pyrrolidinylmethoxy)-3-hydroxymethylpyridine

The product of Example 42a (100 mg, 0.28 mM) was treated with NaBH₄ (52.46 mg, 1.4 mM) in absolute EtOH (35 mL) and stirred at room temperature for 1.5 hours. The solvent was removed under vacuum and the colorless residue was partitioned between 0.5 N HCl (20 mL) and EtOAc (50 mL). The aqueous layer was extracted with EtOAc (2×50 mL) and the extracts were combined and washed with brine. The organic phase was dried (MgSO₄) and concentrated under vacuum to provide the title compound (90 mg, 90%).MS (DCI-NH₃) m/z 360 (M+NH₄)⁺; ¹H NMR (300 MHz, MeOH-d₄) δ1.48 (m, 9H), 1.75-1.9 (m, 1H), 2.0-2.2 (m, 1H), 2.65-2.8 (m, 1H), 3.15-3.65 (m, 4H), 4.0-4.18 (m, 2H), 4.52 (s, 2H), 7.58 (d, J=3 Hz, 1H) 7.95 (d, J=3 Hz, 1H).

### 43b. 2-Chloro-3-hydroxymethyl-5-(3-(S)-pyrrolidinylmethoxy)pyridine hydrochloride

The product of Example 43a was converted to the title compound according to the procedure of Example 42b. MS (DCI-NH₃) m/z 243 (M+H)⁺; ¹H NMR (300 MHz, MeOH-d₄) δ1.9-2.05 (m, 1H), 2.2-2.35 (m, 1H), 2.8-3.0 (m, 1H), 3.18-3.6 (m, 4H), 4.08-4.25 (m, 2H), 4.65 (s, 2H), 7.62 (d, J=3 Hz, 1H)7.98 (d, J=3 Hz, 1H); Calculated analysis for C₁₁H₁₅ClN₂O₂.HCl.0.5 H₂O: C, 45.85; H, 5.95; N, 9.72. Found C, 45.83; H, 5.56; N, 9.55.

### EXAMPLE 44

### 2-Chloro-3-(methoxyiminomethyl)-5-(3-(S)-pyrrolidinylmethoxy)pyridine hydrochloride

### 44a. 2-Chloro-5-(1-[BOC]-3-(S)-pyrrolidinylmethoxy)-3-(methoxyiminomethyl)pyridine

A solution of the product of Example 42a (100 mg, 0.28 mmol) in 80% EtOH-H₂O (20 mL) was treated with methoxylamine hydrochloride (116.9 mg, 1.4 mmol) and NaOAc (190.5 mg, 1.4 mmol), and heated for 2 hours at 60° C. The solvent was removed under vacuum, and the residue was partitioned between EtOAc (30 mL) and brine (10 mL). The organic phase was dried over MgSO₄ and concentrated under vacuum to leave a white crystalline solid (80 mg, 74%) MS (DCI-NH₃)m/z 387 (M+NH₄)⁺; ¹H NMR (300 MHz, CDCl₃) δ1.7-1.9 (m, 1H), 2.0-2.2 (m, 1H), 2.6-2.8 (m, 1H), 3.1-3.7(m, 4H), 3.9-4.05 (m, 2H), 4.05 (s, 3H), 7.68 (d, J=3 Hz, 1H) 8.08 (d, J=3 Hz, 1H), 8.35 (s, 1H).

### 44b. 2-Chloro-3-(methoxyiminomethyl)-5-(3-(S)-pyrrolidinylmethoxy)pyridine hydrochloride

The product of Example 44a was converted to the HCl salt as described for Example 42b. MS (DCI-NH₃) m/z 270 (M+H)⁺; ¹H NMR (300 MHz, CDCl₃) δ2.0-2.15 (m, 1H), 2.3-2.45 (m, 1H), 2.9-3.08 (m, 1H), 3.05-3.7 (m, 4H), 4.02 (s, 3H), 4.15-4.25 (m, 2H), 7.88 (d, J=3 Hz, 1H) 8.22 (d, J=3 Hz, 1H), 8.42(s, 1H); Calulated analysis for C₁₂H₁₆ClN₃O₂.HCl, C,47.07; H, 5.60; N, 13.72. Found: C, 46.83; H, 5.61; N, 13.44.

### EXAMPLE 45

### 2-Chloro-5-(3-(R)-pyrrolidinylmethoxy)-3-pyridinecarboxaldehyde hydrochloride

### 45a. 2-Chloro-5-(1-[BOC]-3-(R)-pyrrolidinylmethoxy)-3-pyridinecarboxaldehyde

The procedure of Example 42a was used, substituting (R)-5-bromo-6-chloro-3-(1-[BOC]pyrrolidinyl-3-methoxy)pyridine from Example 35a for the (S) enantiomer therein. The title product was obtained in 51% yield.MS (DCI-NH₃) m/z 358 (M+H)⁺; ¹H NMR (300 MHz, MeOH-d₄) δ1.48 (m, 9H), 1.7-1.9 (m, 1H), 2.05-2.2 (m, 1H), 2.6-2.8 (m, 1H), 3.15-3.65 (m, 4H), 3.95-4.15 (m, 2H), 4.52 (s, 2H), 7.65 (d, J=3 Hz, 1H) 8.05 (d, J=3 Hz, 1H).

### 45b. 2-Chloro-5-(3-(R)-pyrrolidinyl-3-methoxy)-3-pyridinecarboxaldehyde hydrochloride

By converting the product of Example 45a to the salt according to the procedure for Example 42b, the title compound is formed.

### EXAMPLE 46

### 2-Chloro-3-hydroxymethyl-5-(3-(R)-pyrrolidinylmethoxy)pyridine hydrochloride

### 46a. 2-Chloro-5-(1-[BOC]-3-(R)-pyrrolidinylmethoxy)-3-hydroxymethylpyridine

The procedure of Example 43a was used, substituting 2-chloro-5-(1-[BOC]-3-(R)-pyrrolidinylmethoxy)-3-pyridinecarboxaldehyde from Example 45a for the S-enantiomer described therein. MS (DCI-NH₃)m/z 360 (M+H)⁺; ¹H NMR (300 MHz, MeOH-d₄) δ1.48 (s, 9H), 1.72-1.9 (s, 1H), 2.0-2.2 (m, 1H), 2.6-2.8 (m, 1H), 3.15-3.65 (m, 4H), 4.0-4.12 (m, 2H), 7.58 (d, J=3Hz, 1H) 7.95 (d, J=3 Hz, 1H).

### 46b. 2-Chloro-3-hydroxymethyl-5-(3-(R)-pyrrolidinylmethoxy)pyridine hydrochloride

The product of Example 46a was converted to the salt according to the procedure described for Example 42b. MS (DCI-NH₃) m/z 360 (M+NH₄)⁺; ¹H NMR (300 MHz, MeOH-d₄) δ1.48 (s, 9H), 1.72-1.9 (s, 1H), 2.0-2.2 (m, 1H), 2.6-2.8 (m, 1H), 3.15-3.65 (m, 4H), 4.0-4.12 (m, 2H), 7.58 (d, J=3 Hz, 1H) 7.95 (d, J=3 Hz, 1H). Calculated analysis for C₁₁H₁₅ClN₂O₂.HCl: C, 47.33; H, 5.78; N, 10.03. Found: C, 47.23; H, 5.59; N, 9.74; [α]_{D} +7.69 (c 0.13, MeOH).

### EXAMPLE 47

### 2-chloro-5-(3-(R)-pyrrolidinylmethoxy)-3-pyridinecarbaldoxime hydrochloride

### 47a. 2-Chloro-5-(1-[BOC]-3-(R)-pyrrolidinylmethoxy)-3-pyridinecarbaldoxime

The procedure of Example 44a was used, substituting hydroxylamine hydrochloride for methoxylamine hydrochloride, and (R)-2-chloro-5-(1-[BOC]pyrrolidinyl-3-methoxy)-3-pyridinecarboxaldehyde for the S-enantiomer, described therein. The title compound was obtained in 64% yield.MS (DCI-NH₃) m/z 373 (M+NH₄)⁺; ¹H NMR (300 MHz, MeOH-d₄) δ1.75-1.9 (m, 1H), 2.02-2.2 (m, 1H), 2.6-2.8 (m, 1H), 3.12-3.65 (m, 4H), 4.0-4.15 (m, 2H), 7.8 (m, 1H) 8.1 (m, 1H). 8.32 (s, 1H).

### 47b. 2-Chloro-5-(3-(R)-pyrrolidinylmethoxy)-3-pyridinecarbaldoxime hydrochloride

The product of Example 47a was converted to the HCl salt according to the procedure of Example 42b. MS (DCI-NH₃) m/z 256 (M+H)⁺; ¹H NMR (300 MHz, MeOH-d₄) δ1.82-2.05 (m, 1H), 2.2-2.35 (m, 1H), 2.8-2.95 (m, 1H), 3.15-3.6 (m, 4H), 4.05-4.25 (m, 2H), 7.82 (d, J=3 Hz, 1H) 8.12 (d, J=3 Hz, 1H), 8.32 (s, 1H); Calculated analysis for C₁₁H₁₄ClN₃O₂.HCl: C, 45.22; H, 5.17; N, 14.38. Found 45.34; H, 5.21; N, 14.07; [α]_{D} +1.8 (c 0.17, MeOH).

### EXAMPLE 48

### 3-Aminomethyl-2-chloro-5-(3-(R)-pyrrolidinylmethoxy)pyridine bis(4-methylbenzenesulfonate)

### 48a. 2-Chloro-5-(1-[BOC]-3-(R)-pyrrolidinylmethoxy)pyridine-3-methyl methanesulfonate

A solution of the product of Example 46a (180 mg, 0.5 mmol) in THF (10 mL) was cooled to 0° C. and treated with triethylamine (0.7 mL, 0.5 mmol) and methanesulfonyl chloride (0.39 mL, 0.5 mmol). The solution was allowed to warm to room temperature and stirred for 10 hours. The reaction mixture was partitioned between brine (10 mL) and ethyl acetate (40 mL). The organic layer was separated and washed successively with 5% NaHCO₃, brine, then dried (MgSO₄) and concentrated to an oil (190 mg, 87%). MS (DCI-NH₃) m/z 438 (M+NH₄)⁺; ¹H NMR (300 MHz, CDCl₃) δ1.48 (s, 9H), 1.7-1.88 (m, 1H), 2.0-2.2 (m, 1H), 2.6-2.75 (s, 3H), 3.1-3.7 (m, 4H), 3.9-4.05 (m, 2H), 5.38 (s, 2H), 7.38 (d, J=3 Hz, 1H), 8.08 (d, J=3 Hz, 1H).

### 48b. 3-Azidomethyl-2-chloro-5-(1-[BOC]-3-(R)-pyrrolidinylmethoxy)pyridine

A solution of the product of Example 48a (190 mg, 0.43 mmol) in anhydrous DMF (3 mL) was treated with NaN₃ (280 mg, 4.3 mM) at 65° C. for 2 hours. The reaction mixture was diluted with brine (20 mL) and extracted in EtOAc (3×50 mL). The organic phase was dried (MgSO₄), filtered, and concentrated under vacuum to leave a yellow oil (165 mg, 100%) which was used directly in the next step. MS (DCI-NH₃) m/z 385 (M+NH₄)⁺; ¹H NMR (300 MHz, MeOH-d₄) δ1.48 (s, 9H), 1.75-1.92 (s, 1H), 2.05-2.2 (m, 1H), 2.62-2.78 (m, 1H), 3.15-3.65 (m, 4H), 4.0-4.12 (m, 2H), 4.55 (s, 2H), 7.55 (d, J=3 Hz, 1H) 7.95 (d, J=3 Hz, 1H).

### 48c. 3-(BOC-amino)methyl-2-chloro-5-(1-[BOC]-3-(R)-pyrrolidinyl-3-methoxy)pyridine

A solution of the azide product of Example 48b (165 mg, 0.43 mmol) in THF (10 mL) and H₂O (0.2 mL) was treated with triphenylphosphine (124 mg, 0.47 mM) at 70° C. for 1 hour. The solution was cooled to room temperature and di-t-butyl dicarbonate (103.23 mg, 0.47 mmol) was added and the solution was stirred for 16 hours. The solvent was removed under vacuum, and the residue was purified on silica gel (2:1 hexanes-EtOAc) to provide the title compound (131 mg, 66%). MS (DCI-NH₃) m/z 459 (M+NH₄)⁺; ¹H NMR (300 MHz, MeOH-d₄) δ1.48 (s, 18H), 1.75-1.90 (m, 1H), 2.05-2.2 (m, 1H), 2.6-2.8 (m, 1H), 3.10-3.65 (m, 4H), 3.95-4.05 (m, 2H),7.35 (d, J=3 Hz, 1H) 7.98 (d, J=3 Hz, 1H).

### 48d. 3-Aminomethyl-2-chloro-5-(3-(R)-pyrrolidinylmethoxy)pyridine bis(4-methylbenzenesulfonate)

A solution of the product of Example 48c (130 mg, 0.29 mmol) in EtOAc (10 mL) was treated with p-toluenesulfonic acid monohydrate (118 mg, 0.61 mmol) and the solution was warmed to reflux for 4 hours. The solution was concentrated under vacuum, and the residue was triturated with EtOAc (5 mL) to yield the title compound as a foamy solid (77 mg, 44%). MS (DCI-NH₃) m/z 242 (M+H)⁺; ¹H NMR (300 MHz, MeOH-d₄) δ1.86-2.05, (m, 1H), 2.2-2.35 (m, 1H), 2.8-2.98 (m, 1H), 3.15-3.6 (m, 4H), 4.1-4.25 (m, 2H), 4.28 (s, 1H), 7.23 (d, J=8.25 Hz, 4H) 7.68 (d, J=8.25 Hz, 4H), 7.66 (d, J=3 Hz, 1H), 8.28 (d, J=3 Hz, 1H); Calculated analysis for C₁₁H₁₆ClN₃O .2C₇H₈O₃S: C, 51.23; H, 5.50; N, 7.17. Found: C, 51.13; H, 5.43; N, 6.84.

### EXAMPLE 49

### 2-Chloro-5-(3-(R)-pyrrolidinylmethoxy)pyridine-3-nitrile 4-methylbenzenesulfonate

### 49a. 2-Chloro-3-cyano-5-(1-BOC-3-(R)-pyrrolidinylmethoxy)pyridine

To a flame-dried flask purged with nitrogen is added 5-bromo-6-chloro-3-(1-[BOC]-3-(R)-pyrrolidinylmethoxy)pyridine from Example 35a, Zn(CN)₂, and tetrakis(triphenylphosphine)palladium(0). Degassed DMF is added, and the mixture is heated to 80° C. for 16 hours. The mixture is partitioned with aqueous NaHCO₃ and EtOAc, and the organic phase is dried and concentrated. The residue is purified on silica and converted to provide the title compound.

### 49b. 2-Chloro-5-(3-(R)-pyrrolidinylmethoxy)pyridine-3-nitrile 4-methylbenzenesulfonate

The product of Example 49a is subjected to the procedure of Example 48d to provide the title compound.

### EXAMPLE 50

### 3-(pyrrolidinyl-3-methoxy)pyridine dihydrochloride

Racemic 1-pyrrolidine-3-methanol (J. Chem. Soc. 1959, 851-854) was carried through the procedures of Examples 34b-d, substituting 3-hydroxypyridine for the 2-chloro-3-methyl-5-hydroxypyridine of Example 34d. The product was converted to th& salt according to the procedure of Example 42b to afford the title compound. mp 168-171° C., ¹H NMR (300 MHz, D₂O) δ2.01 (m, 1H), 2.32 (m, 1H), 2.99 (m, 1H), 3.2-3.6 (m, 3 H), 4.31 (d of AB quartet, J=9, 6 Hz, 2H), 7.97 (dd, J=9, 5 Hz, 1H), 8.16 (ddd, J=9, 3, 1 Hz, 1H), 8.41 (d, J=5 Hz, 1H), 8.49 (d, J=3 Hz, 1H).

### EXAMPLE 51

### 3-(1-methylpyrrolidinyl-3-methoxy)pyridine dihydrochloride

The product of Example 50 was methylated according to the procedure of Example 36a and the product was converted to the salt by the procedure of Example 42b. ¹H NMR (300 MHz, D₂O) δ2.0-2.5 (m, 2H), 2.98 (s, 3H), 3.0-3.4 (m, 3H), 3.7-4.0 (m, 2H), 4.30 (m, 2H), 7.96 (dd, J=9, 6 Hz, 1H), 8.16 (br d, J=9 Hz, 1H), 8.40 (d, J=6 Hz, 1H), 8.49 (t, J=3 Hz, 1H).

### EXAMPLE 52

### 2-Methyl-3-(3-(R)-pyrrolidinylmethoxy)pyridine dihydrochoride

The product of Example 34c was condensed with 2-methyl-3-hydroxypyridine according to the procedure of Example 34d. The resulting 2-methyl-3-(1-[BOC]-3-(R)-pyrrolidinylmethoxy)pyridine was deprotected and converted to the salt according to the procedure of Example 42b to provide the title compound as a hygroscopic oil. MS (CI-NH₃) m/z 193 (M+H)⁺; ¹H NMR (300 MHz, CD₃OD) δ2.02 (m, 1H), 2.34 (m, 1H), 2.69 (s, 3H), 3.00 (m, 1H), 3.20-3.64 (m, 3H), 4.13 (m, 1H), 4.23 (m, 1H), 4.35 (m, 2H), 7.86 (dd, J=8, 6 Hz, 1H), 8.16 (d, J=8 Hz, 1H), 8.27 (d, J=6 Hz, 1H).

### EXAMPLE 53

### 5-(N-Benzoylamino)methyl)-6-chloro-3-(3-(R)-pyrrolidinylmethoxy)pyridine hydrochloride

### 53a. 5-(N-Benzoylamino)methy1)-6-chloro-3-(-(BOC)-3-(R)-pyrrolidinylmethoxy)pyridine

3-Azidomethyl-2-chloro-5-(1-[BOC]-3-(R)-pyrrolidinylmethoxy)pyridine from Example 48b is treated with an equimolar amount of triphenylphosphine in moist THF according to the procedure of Example 48c. The solution is cooled, and treated with triethylamine and benzoyl chloride. The mixture is concentrated, and the residue purified by chromatography on silica to provide the title compound.

### 53b. 5-(N-Benzoylamino)methyl)-6-chloro-3-(3-(R)-pyrrolidinylmethoxy)pyridine hydrochloride

The product of Example 53a is treated with HCl in EtOAc, and the precipitate crystallized to afford the title compound.

### EXAMPLE 54

### 5-bromo-6-fluoro-3-(3-(S)-pyrrolidinylmethoxy)pyridine dihydrochloride was prepared in the following manner.

54a. First, 5-Hydroxy-3-bromo-2-(4-nitrophenylazo)pyridine was prepared as follows. 5-bromo-3-pyridinol and potassium hydroxide (Fisher Scientific) is dissolved in water (200 ml). A suspension of p-nitrobenzene diazonium tetrafluoroborate (J. Org. Chem., Vol. 44, No 9. 1979 p 1572-1573) is added, and the mixture is stirred for 1 hour, diluted with acetic acid (50 ml) and filtered. The crude product is allowed to dry in air, then is chromatographed (silica gel; CDCl₃/MeOH, 95:5 to 90:10) to provide 5.45 g (33.7%) of the title compound. MS (DCl/NH₃) m/e 323-325 (M+H)⁺. ¹H NMR (D6DMSO, 300 MHz) δ8.48-8.43 (m, 2H), 8.21-8.20 (d, J=2.37 Hz, 1H), 8.09-8.06 (m, 2H), 7.72-7.71 (d, J=2.37 Hz, 1H)

54b. Then, 5-Hydroxy-3-bromo-2-aminopyridine was prepared as follows. The compound from step 54a above (5.0 g 15.8 mmol) and tin chloride (Aldrich 25 g, 111 mmol) are suspended in conc. HCl (250 ml ) and, methanol (150 ml) and heated-to reflux for 1 hour. The mixture is cooled to 0° C. and then filtered. The solution is neutralized with sodium bicarbonate(180 g) and extracted with ethyl acetate (4×200 ml). The extracts are washed with brine, dried (MgSO₄), and concentrated. The residue is chromatographed (silica gel; CDCl₃/MeOH/NH₄OH, 95:5:.05 to 9:10:1) to afford 3.3 g of the title compound along with substantial amount of tin chloride. MS (DCl/NH₃) m/e 189, 191 (M+H)⁺. ¹H NMR (DMSO-d₆, 300 MHz) δ7.57-7.56 (d, J=2.6 Hz, 1H), 7.43-7.42 (d, J=2.6 Hz, 1H)

54c. 3-bromo-2-fluoro-5-hydroxypyridine was then prepared as follows.

The compound from step 54b above (3.0 g 15.9 mmol) is dissolved in 50 ml of HF pyridine (Aldrich) and cooled to 0° C. under nitrogen and sodium nitrite (1.09 g 15.8 mmol) is added in portions over 20 minutes. The reaction is heated to 50° C. for one hour, cooled to 0° C. and then basified with 20% sodium hydroxide. The aqueous phase is washed with methylene chloride (5×100 ml), neutralized with HCl (pH=7), and extracted with ethyl acetate (5×100 ml). These extracts are dried (MgSO₄), filtered, and concentrated in vacuo to yield the title compound as a tan solid. MS (DCI/NH₃) m/e 192-194 (M+H)⁺. ¹HNMR (DMSO-d₆, 300 MHz) δ9.38 (d, J=2.6 Hz, 1H), 9.20-9.19 (d, J=2.6 Hz, 1H).

54d. 3-Bromo-2-fluoro-5-(1-BOC-3-(S)-pyrrolidinylmethoxy)pyridine was then prepared as follows.

A sample of 1-BOC-3-(S)-pyrrolidinemethanol, prepared as described above, and of 3-bromo-2-fluoro-5-hydroxypyridine, prepared as in step b above, are reacted with triphenylphosphine and DEAD in THF at room temperature for 16 hours, to give the title compound.

54e. 3-Bromo-2-fluoro-5-(3-(S)-pyrrolidinylmethoxy)pyridine dihydrochloride was prepared as follows.

The BOC group is removed from the compound of step 54d by treatment with TFA in methylene chloride to give the free base of the title compound. The base is converted to the salt by treatment with hydrogen chloride saturated ethanol. The solvents are removed under vacuum to give the title compound.

### EXAMPLE 55

3-Bromo-2-fluoro-5-(3-(R)-pyrrolidinylmethoxy)pyridine dihydrochloride is prepared by the procedures of Examples 54d and 54e, substituting 1-BOC-3-(R)-pyrrolidinemethanol for the (S) enantiomer described therein.

### EXAMPLES 56-61

By analogy to the procedures of Examples 42, 48, and 53, utilizing the appropriate 3-bromo-5-(1-BOC-3-pyrrolidinylmethoxy)pyridine from Examples 27a, 35a, 41a, 54d or 55, and replacing benzoyl chloride of Example 53a with the acylating agent shown in Table 1 below, the desired compounds having R¹⁷ and R¹⁸ as described in Table 1 can be prepared. For the structures above Table 1, m and n are each integers of from 1 to 6, wherein the sum of n+m≦7, and R¹ is selected from the group consisting of hydrogen, C₁-C₆ alkyl, aryl, propargyl, cycanomethyl, cycloalkyl, non-aromatic heterocycles and cycloalkyl alkyl.

TABLE 1

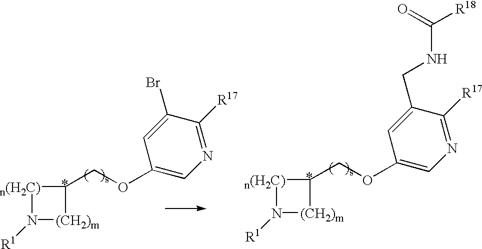

| Ex.No | * | R¹⁷ | Acylating agent | R¹⁸ |
|---|---|---|---|---|
| 56 | (R) | F | acetic anhydride | acetyl |
| 57 | (S) | F | 6-chlorohexanoyl | 6-chlorohexanoyl |
| | | | chloride | |
| 58 | (R) | Cl | ethyl formate | H |
| 59 | (S) | Cl | dimethyl dicarbonate | methoxyl |
| 60 | (R) | H | furoyl chloride | furanyl |
| 61 | (S) | H | 3-nicotinoyl chloride | 3-pyridyl |

### EXAMPLES 62-67

Following the procedure of Example 53, replacing the 5-bromo-6-chloro-3-(1-methyl-3-(S)-pyridinylmethoxy)pyridine starting material thereof with the starting materials having the structures shown above for Table 1 with substituents as in Table 2 below, and replacing the benzoyl chloride of step 53a with the acylating reagent shown in Table 2, the desired compounds 62-67 having R¹⁷ and R¹⁸ as described in Table 2 can be prepared.

TABLE 2

| Ex. | | | | |
|---|---|---|---|---|
| No | * | R¹⁷ | Acylating agent | R¹⁸ |
| 62 | (R) | F | 3-phenylpropionoyl | 2-phenylethyl |
| | | | chloride | |
| 63 | (S) | F | 4-chlorobenzoyl | 4-chlorophenyl |
| | | | chloride | |
| 64 | (R) | Cl | 3-nitrobenzoyl chloride | 3-nitrophenyl |
| 65 | (S) | Cl | 2-pyrrole-carboxylic | 2-pyrrolyl |
| | | | acid + EDC | |
| 66 | (R) | H | 5-nitro-2-furan- | 5-nitrofuranyl |
| | | | carboxylic acid + EDC | |
| 67 | (S) | H | 2-pyrazine-carboxylic | 2-pyrazinyl |
| | | | acid + EDC | |

### EXAMPLE 68

3-benzoyl-2-chloro-5-(3-(S)-pyrrolidinylmethoxy)pyridine hydrochloride was prepared as follows.

68a. 3-benzoyl-2-chloro-5-(1-BOC-3-(R)-pyrrolidinylmethoxy)pyridine was first prepared as follows.

The 2-chloro-3-cyano-5-(1-BOC-3-(R)-pyrrolidinylmethoxy)pyridine of example 49a in anhydrous ether at 0° C. is treated with 1.5 equivalents of phenylmagnesium bromide in ether and stirring is maintained at 0 to 35° C. until the nitrile is largely consumed. The solvent is evaporated and the residue is treated with 2M aqueous potassium hydrogen sulfate to hydrolyze the intermediate imine. The solution is made basic with potassium carbonate and extracted with ethyl acetate. The combined extracts are dried (Na₂SO₄) and concentrated to a residue which is chromatographed (silica gel) to afford the title compound.

68b. 3-benzoyl-2-chloro-5-(3-(S)-pyrrolidinylmethoxy)pyridine hydrochloride was then prepared as follows.

3-benzoyl-2-chloro-5-(1-BOC-3-(S)-pyrrolidinylmethoxy)pyridine from step 68a is dissolved in methylene chloride (10 mL). The mixture is cooled to 0° C., TFA (10 mL) is added and the reaction is stirred for 45 minutes as it warms to room temperature. The mixture is concentrated in vacuo and taken up in a minimum amount of H₂O. The aqueous mixture is basified with 15% NaOH and extracted with CH₂Cl₂ (200 mL), which is dried (MgSO₄) and concentrated. The residue is chromatographed (silica gel) to afford the free amine. The isolated free amine is taken up in a minimum amount of Et₂O, cooled to 0° C., and treated with HCl in ethanol to afford the hydrochloride salt.

### EXAMPLES 69-72

Following the procedure of Example 68, replacing the 2-chloro-3-cyano-5-(1-BOC-3-(R)-pyrrolidinylmethoxy)pyridine with the starting material compounds shown in Table 3 and replacing the phenylmagnesium bromide reagent thereof with a R¹⁹—Mg—Br Grignard reagent or a R¹⁹—Li reagent(wherein R¹⁹ is specified in Table 3 below), the desired compounds 69-72 having R¹⁷ and R¹⁹ as described in Table 3 can be prepared. For the structures above Table 3, m and n are each integers of from 1 to 6, wherein the sum of n+m≦7; s is an integer of 0 or 1; and R¹ is selected from the group consisting of hydrogen, C₁-C₆ alkyl, aryl, propargyl, cyanomethyl, cycloalkyl, non-aromatic heterocycles and cycloalkyl alkyl.

TABLE 3

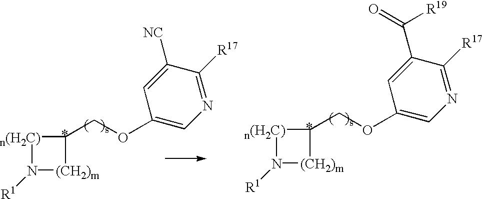

| Ex.No. | * | R¹⁷ | R¹⁹ |
|---|---|---|---|
| 69 | (R) | F | n-hexyl |
| 70 | (S) | Cl | 3-quinolinyl |
| 71 | (R) | H | 2-naphthyl |
| 72 | (S) | H | 4-methyl-1-naphthyl |

### EXAMPLES 73-78

Following the procedure of Example 68, replacing the 5-cyano-6-chloro-3-(1-BOC-3-(R)-pyrrolidinylmethoxy)pyridine with the starting material compounds of the structures shown above Table 3 and replacing the phenylmagnesium bromide reagent thereof with a R¹⁹—Mg—Br Grignard reagent as indicated in Table 4 below, the desired compounds 73-78 having R¹⁷ and R¹⁹ as described in Table 4 can be prepared. For the structures above Table 3, m and n are each integers of from 1 to 6, wherein the sum of n+m≦7, s is an integer of 0 or 1; and R¹ is selected from the group consisting of hydrogen, C₁-C₆ alkyl, aryl, propargyl, cyanomethyl, cycloalkyl, non-aromatic heterocycles and cycloalkyl alkyl.

TABLE 4

| Ex. No | * | R¹⁷ | R¹⁹ |
|---|---|---|---|
| 73 | (R) | F | 3-pyridinyl |
| 74 | (S) | F | 5-pyrimidinyl |
| 75 | (R) | Cl | 3-pyridazinyl |
| 76 | (S) | Cl | 2-thienyl |
| 77 | (R) | H | phenylmethyl |
| 78 | (S) | H | 2-(4-methoxy- |
| | | | phenyl)ethyl |

### EXAMPLES 79-82

Following the procedure of Example 2b, replacing the styrene starting material thereof with the starting material compounds shown in Table 5, then hydrogenating the product thereof with palladium on charcoal, the desired compounds 79-82 having R¹⁷ and R²⁰ as described in Table 5 can be prepared. For the structures above Table 5, m and n are each integers of from 1 to 6, wherein the sum of n+m≦7, s is an integer of 0 or 1; and R¹ is selected

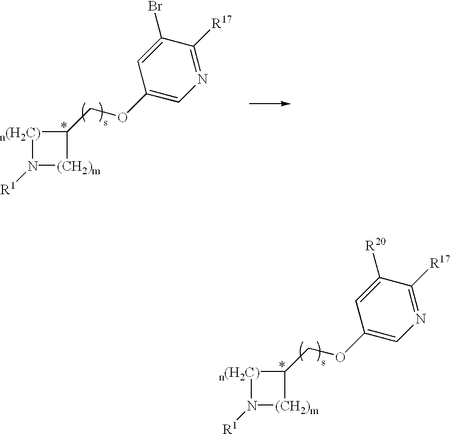

from the group consisting of hydrogen, C₁-C₆ alkyl, aryl, propargyl, cyanomethyl, cycloalkyl, non-aromatic heterocycles and cycloalkyl alkyl.

TABLE 5

| Ex. No | * | R¹⁷ | Starting material | R²⁰ |
|---|---|---|---|---|
| 79 | (S) | H | 5-carbomethoxy-3 | 2-(5-carbomethoxy- |
| | | | vinylpyridine | pyridinyl)ethyl |
| 80 | (S) | H | 5-bromo-3- | 2-(5-bromo-pyridinyl) |
| | | | vinylpyridine | ethyl |
| 81 | (S) | H | 6-amino-5-bromo- | 2-(6-amino-5-bromo- |
| | | | 3-vinylpyridine | pyridinyl)ethyl |
| 82 | (S) | H | 5-bromo-6- | 2-(5-bromo-6- |
| | | | methylamino- | methylamino |
| | | | 3-vinylpyridine | pyridinyl)ethyl |

### EXAMPLES 83-91

Following the procedure of Example 12, replacing the 3-bromo-5-(1-methyl-3-(S)-pyrrolidinyloxy)-pyridine thereof with the starting material compound shown in Table 6 having the structure as shown above Table 5 and replacing the 3-pyridinyltributyltin reagent thereof with the reagent shown in Table 6, the desired compounds 83-91 having R¹⁷ and R²⁰ as described in Table 6 can be prepared. For the structures above Table 5, m and n are each integers of from 1 to 6, wherein the sum of n+m≦7; s is an integer of 0 or 1; and R¹ is selected from the group consisting of hydrogen, C₁-C₆ alkyl, aryl, propargyl, cyanomethyl, cycloalkyl, non-aromatic heterocycles and cycloalkyl alkyl.

TABLE 6

| Ex. No | * | R¹⁷ | reagent | R²⁰ |
|---|---|---|---|---|
| 83 | (R) | H | 5-carbomethoxy-3- | 5-carboxy-3- |
| | | | pyridinyltributyltin¹ | pyridinyl |
| 84 | (S) | H | 5-carbomethoxy-3- | 5-formyl-3-pyridinyl |
| | | | pyridinyltributyltin² | |
| 85 | (R) | H | 5-hydroxymethyl- | 5-hydroxymethyl-3- |
| | | | 3-pyridinyltributyltin | pyridinyl |
| 86 | (S) | H | 2,4-dimethoxy-5- | 2,4-dimethoxy-5- |
| | | | pyrimidinyltributyl tin | pyrimidinyl |
| 87 | (R) | H | 2-chloro-3- | 2-chloro-3-thienyl |
| | | | thienyltributyltin | |
| 88 | (S) | H | 2-cyano-3- | 2-cyano-3-thienyl |
| | | | thienyltributyltin | |
| 89 | (S) | H | 4-methyl-3- | 4-methyl-3-thienyl |
| | | | thienyltributyltin | |
| 90 | (S) | H | 4-hydroxymethyl- | 4-hydroxymethyl-5- |
| | | | 5-carbomethoxy-3- | carbomethoxy-3-thienyl |
| | | | thienyltributyltin | |
| 91 | (S) | H | 4-ethoxymethoxy- | 4-methoxymethoxy- |
| | | | 5-carbomethoxy-3- | 5-carbomethoxy-3-thienyl |
| | | | thienyltributyltin | |

1=After following the procedures of Example 12, with substitutions as indicated, the carbomethoxy group is hydrolyzed with base as additional step in this preparation.

2=After following the procedures of Example 12, with substitutions as indicated, the additional steps are necessary: the carbomethoxy group is hydrolyzed with base; the resulting free acid is reduced to the alcohol with LAH, and the resulting alcohol is oxidized to the aldehyde with Jones' or Collins' reagents.

### EXAMPLES 92-94

Following the procedure of Example 2, replacing the styrene starting material thereof with the starting material compounds shown in Table 7 having the structures as shown above Table 5, then hydrogenating the product thereof with palladium on charcoal the desired compounds 92-94 having R¹⁷ and R²⁰ as described in Table 7 can be prepared. For the structures above Table 5, m and n are each integers of from 1 to 6, wherein the sum of n+m≦7; s is an integer of 0 or 1; and R¹ is selected from the group consisting of hydrogen, C₁-C₆ alkyl, aryl, propargyl, cyanomethyl, cycloalkyl, non-aromatic heterocycles and cycloalkyl alkyl.

TABLE 7

| Ex. No | * | R¹⁷ | Starting material | R²⁰ |
|---|---|---|---|---|
| 92 | (S) | H | 4-methyl-3- | 2-(4-methyl-3- |
| | | | vinylbenzene | phenyl)ethyl |
| 93 | (S) | H | 4-methoxy-3- | 2-(4-methoxy-3- |
| | | | vinylbenzene | phenyl)ethyl |
| 94 | (S) | H | 4- | 2-(4-trifluoromethyl- |
| | | | trifluoromethyl- | 3-phenyl)ethyl |
| | | | 3-vinylbenzene | |

### EXAMPLES 95-98

Following the procedure of Example 15, replacing the 3-bromo-5-(1-methyl-3-(S)-pyrrolidinyloxy)-pyridine thereof with the starting material compound shown in Table 8 and replacing the phenylboronic acid reagent thereof with the reagent shown in Table 8 of the structure as shown above Table 5, the desired compounds 95-98 having R¹⁷ and R²⁰ as described in Table 8 can be prepared. For the structures above Table 5, m and n are each integers of from 1 to 6, wherein the sum of n+m≦7; s is an integer of 0 or 1; and R¹ is selected from the group consisting of hydrogen, C₁-C₆ alkyl, aryl, propargyl, cyanomethyl, cycloalkyl, non-aromatic heterocycles and cycloalkyl alkyl.

TABLE 8

| Ex. No | * | R¹⁷ | reagent | R²⁰ |
|---|---|---|---|---|
| 95 | (R) | H | 2-hydroxy-1- | 2-hydroxy-1- |
| | | | naphthylboronic acid | naphthyl |
| 96 | (S) | H | 4′-nitro-4- | 4′-nitro-4- |
| | | | biphenylboronic acid | biphenyl |
| 97 | (R) | H | 4′-fluoro-4- | 4′-fluoro-4- |
| | | | biphenylboronic acid | biphenyl |
| 98 | (R) | H | 4′-methyl-4- | 4′-methyl-4- |
| | | | biphenylboronic acid | biphenyl |

### EXAMPLE 99

Compounds of the invention were subjected to in vitro assays against the nicotinic acetylcholine receptor as described below and were found to be effective binders to the receptor. The in vitro protocols for determination of nicotinic acetylcholine channel receptor binding potencies of ligands was determined as follows.

Binding of [³H]-cytisine ([³H]-CYT) to neuronal nicotinic acetylcholine receptors was accomplished using crude synaptic membrane preparations from whole rat brain (Pabreza et al., Molecular Pharmacol., 1990, v. 39, p. 9). Washed membranes were stored at −80° C. prior to use. Frozen aliquots were slowly thawed and resuspended in 20 volumes of buffer (containing: 120 mM NaCl, 5 mM KCl, 2 mM MgCl₂, 2 mM CaCl₂ and 50 mM Tris-Cl, pH 7.4 at 4° C.). After centrifuging at 20,000×g for 15 minutes, the pellets were resuspended in 30 volumes of buffer.

The test compounds were dissolved in water to make 10 mM stock solutions. Each solution was then diluted (1:100) with buffer (as above) and further taken through seven serial log dilutions to produce test solutions from 10⁻⁵ to 10⁻¹¹ M.

Homogenate (containing 125-150 μg protein) was added to triplicate tubes containing the range of concentrations of test compound described above and [³H]-CYT (1.25 nM) in a final volume of 500 μl. Samples were incubated for 60 minutes at 4° C., then rapidly filtered through Whatman GF/B filters presoaked in 0.5% polyethyleneimine using 3×4 mL of ice-cold buffer. The filters are counted in 4 mL of Ecolume® (ICN). Nonspecific binding was determined in the presence of 10 μM (−)-nicotine and values were expressed as a percentage of total binding. IC₅₀ values were determined with a four-parameter non-linear regression and IC₅₀ values were converted to Kᵢ values using the Cheng and Prusoff correction (Kᵢ=IC₅₀/(1+[ligand]/K_{d} of ligand).

The results are detailed in Table 9. Each Example Number corresponds to the synthetic Examples described above. Examples 1-52 in this table are the compounds of the present invention. The lower the Kᵢ value, the more affinity for neuronal nicotinic acetylcholine receptors.

TABLE 9

| Example | |
|---|---|
| No. | Kᵢ (nM) |
| 1 | 46 |
| 2 | 1.9 |
| 3 | 0.83 |
| 4 | 2.8 |
| 5 | 3.1 |
| 6 | 5.6 |
| 7 | 290 |
| 8 | 39 |
| 9 | 17 |
| 10 | 23 |
| 11 | 83 |
| 12 | 56 |
| 13 | 52 |
| 14 | 210 |
| 15 | 19 |
| 16 | 99 |
| 17 | 130 |
| 18 | 41 |
| 19 | 67 |
| 20 | 100 |
| 21 | 48 |
| 22 | 6.1 |
| 23 | 0.99 |
| 25 | 0.042 |
| 26 | 12 |
| 27 | 0.12 |
| 28 | 0.067 |
| 29 | 1.3 |
| 30 | 0.63 |
| 32 | 15 |
| 33 | 0.048 |
| 34 | 0.026 |
| 35 | 0.021 |
| 36 | 2.1 |
| 37 | 0.056 |
| 38 | 0.077 |
| 39 | 1.1 |
| 40 | 1.3 |
| 41 | 0.59 |
| 42 | 4.1 |
| 43 | 1.4 |
| 44 | 1.6 |
| 46 | 0.10 |
| 47 | 0.012 |
| 50 | 0.13 |
| 51 | 8.8 |
| 52 | 5.1 |

### EXAMPLE 100

An in vivo protocol was utilized to determine the effectiveness of nicotinic acetylcholine receptor ligands as analgesic agents in the mouse hot plate paradigm. Separate groups of mice, (n=8/group) were utilized for each dose group. All drugs were administered by the intraperitoneal route of administration. Test drugs were dissolved in water to make a 6.2 mM stock solution. Animals were dosed with this solution (10 mL/kg body weight) for a 62 micromol/kg dose. Lower doses were administered similarly, following serial dilution of the stock solution in half-log increments. Animals were dosed 30 minutes prior to testing in the hot plate. The hot-plate utilized was an automated analgesia monitor (Model #AHP16AN, Omnitech Electronics, Inc. of Columbus, Ohio). The temperature of the hot plate was maintained at 55° C. and a cut-off time of 180 seconds was utilized. Latency until the tenth jump was recorded as the dependent measure. An increase in the tenth jump latency relative to the control was considered an effect.

Table 10 shows the minimally effective dose (MED), among the doses tested, at which a significant effect, as defined above, was observed for the present compounds. The lower the dosage at which the significant effect is observed, the more effective the compound. The data shows that selected compounds of the invention show a significant antinociceptive effect at doses ranging from 6.2 to 62 μmol/kg.

TABLE 10

| | Dosage |
|---|---|
| Example No. | (μmol/kg) |
| 25 | 6.2 |
| 26 | 62 |
| 27 | 19 |
| 29 | 62 |

All references cited are hereby incorporated by reference.

The present invention is illustrated by way of the foregoing description and examples. The foregoing description is intended as a non-limiting illustration, since many variations will become apparent to those skilled in the art in view thereof. It is intended that all such variations within the scope and spirit of the appended claims be embraced thereby.

Changes can be made in the composition, operation and arrangement of the method of the present invention described herein without departing from the concept and scope of the invention as defined in the following claims:

We claim:

1. A compound of the structure 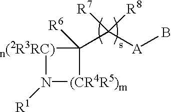
wherein
m and n are each integers of from 1 to 6, and the sum of n+m is from 2 to 7;
s is an integer of 0 to 3;
R¹ is selected from the group consisting of hydrogen, lower alkyl, alkenyl, alkynyl, aryl, aralkyl, aryloxy, arylamino, biaryl, thioaryl, aroyl, heterocyclyl, alkylheterocyclyl, heterocycloyl, cyanomethyl, cycloalkyl, cycloalkenyl and cycloalkylalkyl;
R², R³, R⁴, R⁵, R⁶, R⁷ and R⁸, at each occurrence, are each independently selected from the group consisting of hydrogen, hydroxyl, amino, halogen, lower alkyl, lower alkenyl, lower alkynyl, lower alkoxy, alkenoxy, alkynoxy, thioalkoxy, aliphatic acyl, —CF₃, nitro, cyano, —N(C₁-C₃ alkyl)—C(O)(C₁-C₃ alkyl), —C₁-C₃ alkylamino, alkenylamino, alkynylamino, di(C₁-C₃ alkyl)amino, —C(O)O—(C₁-C₃ alkyl), —C(O)NH—(C₁-C₃ alkyl), —CH═NOH, —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, thioaryl, heterocyclyl, heterocycloyl, alkylaryl, aralkyl, alkylheterocyclyl, sulfonyl, sulfonamido, carbamate, aryloxyalkyl, carboxyl and —C(O)NH(benzyl);
A is selected from the group consisting of —C(R²)(R³)—, —O—, —S—, —N(R¹)—, —SO₂N(R¹)—, —C(O)N(R¹)—, —NR¹C(O)—, —C(O)—, —C(O)O—, —OC(O)— and —N(R¹)SO₂—;
B is selected from the group consisting of heteroaryl and heteroaryl alkyl;
and salts thereof;
with the proviso that when s is 0, the sum of m+n is from 2-5, A=—O— and R¹ is hydrogen or methyl, B is not 3-pyridyl, 5-chloro-3-pyridyl or 2-chloro-3-pyridyl;
and with the further proviso that when R², R³, R⁴, R⁵, R⁶, R⁷ or R⁸ are attached to a carbon which is alpha to a heteroatom, R², R³, R⁴, R⁵, R⁶, R⁷ or R⁸ are not halogen, hydroxyl or amino.

2. A compound of claim 1 further comprising derivatives of said compound selected from the group consisting of esters, carbamates, aminals, amides and pro-drugs thereof.

3. A compound of claim 1 wherein s is 0.

4. A compound of claim 1 of the structure 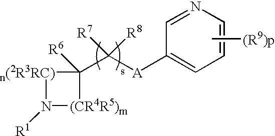
wherein p1 m and n are each integers of from 1 to 6, and the sum of n+m is from 2 to 7;
s is an integer of 0 or 1;
R¹ is selected from the group consisting of hydrogen, lower alkyl, alkenyl, alkynyl, aryl, aralkyl, aryloxy, arylamino, biaryl, thioaryl, aroyl, heterocyclyl, alkylheterocyclyl, heterocycloyl, cyanomethyl, cycloalkyl, cycloalkenyl and cycloalkylalkyl;
R², R³, R⁴, R⁵, R⁶, R⁷ and R⁸, at each occurrence, are each independently selected from the group consisting of hydrogen, hydroxyl, amino, halogen, lower alkyl, lower alkenyl, lower alkynyl, lower alkoxy, alkenoxy, alkynoxy, thioalkoxy, aliphatic acyl, —CF₃, nitro, cyano, —N(C₁-C₃ alkyl)—C(O)(C₁-C₃ alkyl),—C₁-C₃ alkylamino, alkenylamino, alkynylamino, di(C₁-C₃ alkyl)amino, —C(O)O—(C₁-C₃ alkyl), —C(O)NH—(C₁-C₃ alkyl), —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, thioaryl, heterocyclyl, heterocycloyl, alkylaryl, aralkyl, alkylheterocyclyl, sulfonyl, sulfonamido, carbamate, aryloxyalkyl, carboxyl and —C(O)NH(benzyl);
R⁹, at each occurrence, is independently selected from the group consisting of halogen, hydroxyl, lower alkyl, lower alkenyl, lower alkynyl, lower alkoxy, alkenoxy, alkynoxy, thioalkoxy, aliphatic acyl, —CF₃, nitro, cyano, —N(C₁-C₃ alkyl)—C(O)(C₁-C₃ alkyl), —C₁-C₃ alkylamino, alkenylamino, alkynylamino, di(C₁-C₃alkyl)amino, —C(O)O—(C₁-C₃ alkyl), —CH═NOH, —C(O)NH—(C₁-C₃ alkyl), —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, thioaryl, heterocyclyl, heterocycloyl, alkylaryl, aralkyl, alkylheterocyclyl, sulfonyl, sulfonamido, carbamate, aryloxyalkyl, carboxyl, amino and —C(O)NH(benzyl);
A is selected from the group consisting of —C(R²)(R³)—, —O—, —S—, —N(R¹)—, —SO₂N(R¹)—, —C(O)N(R¹)—, —NR¹C(O)—, —C(O)—, —C(O)O—, —OC(O)— and —N(R¹)SO₂—; and
p is an integer of from one to four;
with the proviso that when s=O, A=—O— and p=1, R¹ is hydrogen or methyl, R⁹ is not 5-chloro or 2-chloro;
with the proviso that when A is —O—, s=0, R¹ is hydrogen or methyl and the sum of m+n is from two to five, p is not 0;
and with the further proviso that when R², R³, R⁴, R⁵, R⁶, R⁷ or R⁸ are attached to a carbon which is alpha to a heteroatom, R², R³, R⁴, R⁵, R⁶, R⁷ or R⁸ are not halogen, hydroxyl or amino.

5. A compound of claim 1 of the structure 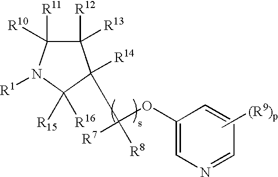
wherein
s is an integer of 0 or 1;
R¹ is selected from the group consisting of hydrogen, lower alkyl, alkenyl, alkynyl, aryl, aralkyl, aryloxy, arylamino, biaryl, thioaryl, aroyl, heterocyclyl, heterocycloyl, alkylheterocyclyl, cyanomethyl, cycloalkyl, cycloalkenyl and cycloalkylalkyl;
R⁷, R⁸, R¹⁰, R¹¹, R¹⁵, and R¹⁶ are independently selected from the group consisting of hydrogen, lower alkyl, lower alkenyl, lower alkynyl, lower alkoxy, alkenoxy, alkynoxy, thioalkoxy, aliphatic acyl, —CF₃, nitro, cyano, —N(C₁-C₃ alkyl)—C(O)(C₁-C₃ alkyl), —C₁-C₃alkylamino, alkenylamino, alkynylamino, di(C₁-C₃ alkyl)amino, —C(O)O—(C₁-C₃ alkyl), —C(O)NH, —(C₁-C₃ alkyl), —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, thioaryl, heterocyclyl, heterocycloyl, alkylaryl, aralkyl, alkylheterocyclyl, sulfonyl, sulfonamido, carbamate, aryloxyalkyl, carboxyl and —C(O)NH(benzyl);
R⁹, at each occurrence, is independently selected from the group —C(O)O—(C₁-C₃ alkyl), —CH═NOH, —C(O)NH—(C₁-C₃ alkyl), —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, thioaryl, heterocyclyl, heterocycloyl, alkylaryl, aralkyl, alkylheterocyclyl, sulfonyl, sulfonamido, carbamate, aryloxyalkyl, carboxyl and —C(O)NH(benzyl);
R¹², R¹³ and R¹⁴ are independently selected from the group consisting of hydrogen, halogen, hydroxyl, lower alkyl, lower alkenyl, lower alkynyl, lower alkoxy, alkenoxy, alkynoxy, thioalkoxy, aliphatic acyl, —CF₃, amino, nitro, cyano, —N(C₁-C₃ alkyl)—C(O)(C₁-C₃ alkyl), —C₁-C₃ alkylamino, alkenylamino, alkynylamino, di(C₁-C₃-alkyl)arnino, —C(O)O—(C₁-C₃ alkyl), —C(O)NH—(C₁-C₃-alkyl), —C(O)N(C₁-C₃ alkyl)₂, haloalkyl, alkoxylcarbonyl, alkoxyalkoxy, earboxaldehyde, carboxamide, cycloalkyl, cycloalkenyl, cycloalkynyl, aryl, aroyl, aryloxy, arylamino, biaryl, thioaryl, heterocyclyl, heterocycloyl, alkylaryl, aralkyl, alkylheterocyclyl, sulfonyl, sulfonamido, carbamate, aryloxyalkyl, carboxyl and —C(O)NH(benzyl);
and p is an integer of from one to four;
with the proviso that when s =0, R¹ is hydrogen or methyl and p=1, R⁹ is not 5-chloro or 2-chloro;
and the further proviso that when s=0, R¹ is hydrogen or methyl, p is not 0.

6. The compound of claim 5 wherein R⁹, at each occurrence, is independently selected from the group consisting of pyridylethenyl, dimethylhexadienyl, chlorophenyl, thienyl, phenyl, aminophenyl, pyridyl, pyrimidyl, octynyl, lower alkyl, —F, —Cl and —Br.

7. The compound of claim 5 wherein R¹ is selected from the group consisting of hydrogen and methyl.

8. The compound of claim 5 wherein s is 1.

9. A compound of claim 1 of the structure 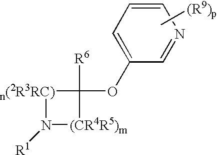
wherein
m and n are each integers of from 1 to 4 and the sum of m and n is 5;
p is an integer of one to four;
R¹ is selected from the group consisting of hydrogen, lower alkyl, alkenyl, alkynyl, aryl, aralkyl, aryloxy, arylamino, biaryl, thioaryl, aroyl, heterocyclyl, heterocycloyl, alkylheterocyclyl, cyanomethyl, cycloalkyl, cycloalkenyl and cycloalkylalkyl;
R², R³, R⁴ and R⁵ are each independently selected from the group consisting of hydrogen, hydroxyl, amino, halogen, lower alkyl, lower alkenyl, lower alkynyl, lower alkoxy, alkenoxy, alkynoxy, thioalkoxy, aliphatic acyl, —CF₃, nitro, cyano, —N(C₁-C₃ alkyl)—C(O)(C₁-C₃ alkyl), —C₁-C₃-alkylamino, alkenylamino, alkynylamino, di(C₁-C₃-alkyl)amino,—C(O)O—(C₁-C₃ alkyl),—C(O)NH—(C₁-C₃-alkyl), —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, thioaryl, alkylaryl, aralkyl, sulfonyl, heterocyclyl, heterocycloyl, alkylheterocyclyl, sulfonamido, carbamate, aryloxyalkyl, carboxyl and —C(O)NH(benzyl);
R⁶ is selected from the group consisting of hydrogen, lower alkyl, lower alkenyl, lower alkynyl, lower alkoxy, alkenoxy, alkynoxy, thioalkoxy, aliphatic acyl, —CF₃, nitro, cyano, —N(C₁-C₃ alkyl)—C(O)(C₁-C₃ alkyl), —C₁-C₃ alkylamino, alkenylamino, alkynylamino, di(C₁-C₃ alkyl)amino, —C(O)O—(C₁-C₃ alkyl), —C(O)NH—(C₁-C₃ alkyl), —C(O)N(C₁-C₃ alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, thioaryl, heterocyclyl, heterocycloyl, alkylaryl, aralkyl, alkylheterocyclyl, sulfonyl, sulfonamido, carbamate, aryloxyalkyl, carboxyl and —C(O)NH(benzyl);
wherein R⁹, at each occurrence, is independently selected from the group consisting of halogen, hydroxyl, lower alkyl, lower alkenyl, lower alkynyl, lower alkoxy, alkenoxy, alkynoxy, thioalkoxy, aliphatic acyl, —CF₃, nitro, amino, cyano, —N(C₁-C₃ alkyl)-CO(C₁-C₃ alkyl), —C₁-C₃ alkylamino, alkenylamino, alkynylamino, di(C₁-C₃ alkyl)amino, —C(O)O—(C₁-C₃ alkyl), —C(O)NH—(C₁-C₃ alkyl),—CH═NOH, —C(O)N(C₁-C₃alkyl)₂, haloalkyl, alkoxylcarbonyl, alkoxyalkoxy, carboxaldehyde, carboxamide, cycloalkyl, cycloalkenyl, cycloalkynyl, aryl, aroyl, aryloxy, arylamino, biaryl, thioaryl, heterocyclyl, heterocycloyl, alkylaryl, aralkyl, alkylheterocyclyl, sulfonyl, sulfonamido, carbamate, aryloxyalkyl, carboxyl and —C(O)NH(benzyl);
and salts thereof;
with the proviso that when R², R³, R⁴ or R⁵ are attached to a carbon which is alpha to a heteroatom, R², R³, R⁴ or R⁵ are not halogen, hydroxyl or amino.

10. The compound according to claim 3 selected from the group consisting of 3-(3-(S)-pyrrolidinyloxy)-5-methylpyridine, 3-(3-(S)-pyrrolidinyloxyl)-5-(2-(4-pyridyl)ethenyl)pyridine, 3-(3-(R)-pyrrolidinyloxy)-5-(2-(4-pyridyl)ethenyl)pyridine, 3-(1-methyl-3-(S)-pyrrolidinyloxy)-5-(2-(4-pyridyl)ethenyl)pyridine, 3-(1-methyl-3-(R)-pyrrolidinyloxy)-5-(2-(4-pyridyl)ethenyl)pyridine, 3-(3-(R)-pyrrolidinylmethoxy)-5-methylpyridine, 3-(3-(S)-pyrrolidinyloxy-5-(5,5-dimethylhexadienyl)pyridine, 3-(3-(S)-pyrrolidinyloxy)-5-(1-octynyl)pyridine, 3-(3-(R)-pyrrolidinyloxy)-5-(1-octynyl)pyridine, 3-(1-methyl-3-(R)-pyrrolidinyloxy)-5-(1-octynyl)pyridine, 3-(3-(S)-pyrrolidinyloxy)-5-(5-pyrimidyl)pyridine, 3-(1-methyl-3-(S)-pyrrolidinyloxy)-5-(3-pyridyl)pyridine, 3-(3-(R)-pyrrolidinyloxy)-5-(5-pyrimidinyl)pyridine, 3-(3-(S)-pyrrolidinyloxy)-5-(3-aminophenyl)pyridine, 3-(3-(S)-pyrrolidinyloxy)-5-phenylpyridine, 3-(1-methyl-3-(S)-pyrrolidinyloxyl)-5-phenylpyridine, 3-(3-(R)-pyrrolidinyloxy)-5-phenylpyridine, 3-(3-(S)-pyrrolidinyloxy)-5-thienylpyridine, 3-(3-(R)-pyrrolidinyloxy)-5-thienylpyridine, 3-(1-methyl-3-(R)-pyrrolidinyloxy)-5-thienylpyridine, 3-(3-(S)-pyrrolidinyloxy)-5-(4-chlorophenyl)pyridine, 3-(3-(S)-pyrrolidinyloxy)-5-bromo-6-chloropyridine, 3-(1-methyl-3-(S)-pyrrolidinyloxy)-5-bromo-6-chloropyridine, 3-(1-methyl-3-(S)-pyrrolidinyloxy)-5-(2-(4-pyridyl)ethenyl)-6-chloropyridine, 3-bromo-2-chloro-5-(3-pyrrolidinylmethoxy)pyridine, 2-bromo-3-chloro-5-(1-methyl-3-pyrrolidinylmethoxy)pyridine, 3-methyl-5-(3-(pyrrolidinyl)methoxy)pyridine, 5-phenyl-3-(3-pyrrolidinylmethoxy)pyridine
and salts thereof.

11. A method for controlling neurotransmitter release in a mammal comprising administering to said mammal a therapeutically effective amount of a compound of claim 1.

12. A pharmaceutical composition comprising:
a compound of claim 1 and pharmaceutically acceptable salts thereof;
in a pharmaceutically acceptable carrier.
